SEQUENCE LISTING (34 sequences)

SEQ ID NO: 1 - protein, 8 aa, Mus musculus
  REGION 1..8: note = misc_feature - anti-hAPRIL heavy chain CDR1 (AA)
GYTFTSYV

SEQ ID NO: 2 - protein, 16 aa, Mus musculus
  REGION 1..16: note = misc_feature - anti-hAPRIL heavy chain CDR2 (AA)
YINPYNDAPKYNEKFK

SEQ ID NO: 3 - protein, 12 aa, Mus musculus
  REGION 1..12: note = misc_feature - anti-hAPRIL heavy chain CDR3 (AA)
GLGYALYYAMDY

SEQ ID NO: 4 - protein, 11 aa, Mus musculus
  REGION 1..11: note = misc_feature - anti-hAPRIL light chain CDR1 (AA)
KASQNVGNNVA

SEQ ID NO: 5 - protein, 7 aa, Mus musculus
  REGION 1..7: note = misc_feature - anti-hAPRIL light chain CDR2 (AA)
SASNRDS

SEQ ID NO: 6 - protein, 10 aa, Mus musculus
  REGION 1..10: note = misc_feature - anti-hAPRIL light chain CDR3 (AA)
QQYNIYPFTF

SEQ ID NO: 7 - DNA, 363 bp, synthetic construct
  misc_feature 1..363: note = Synthetic: VH11 heavy chain variable region (DNA)
  source 1..363: mol_type = other DNA
caggtccagcttgtgcagtctggggctgaggtgaagaagcctggggcctcagtgaaggtttcctgcaaggcttctggatacacattcactagctatgtgatgcattgggtgcgccaggcccccggacaaaggcttgagtggatgggatatattaatccttataatgatgctcctaaatacaatgagaagttcaaaggcagagtcaccattaccagggacacatccgcgagcacagcctacatggagctgagcagcctgagatctgaagacacggctgtgtattactgtgcgagaggcttgggttacgccctttactatgctatggactactggggccaagggaccacggtcaccgtctcctca

SEQ ID NO: 8 - protein, 121 aa, synthetic construct
  REGION 1..121: note = Synthetic: VH11 heavy chain variable region (AA)
QVQLVQSGAEVKKPGASVKVSCKASGYTFTSYVMHWVRQAPGQRLEWMGYINPYNDAPKYNEKFKGRVTITRDTSASTAYMELSSLRSEDTAVYYCARGLGYALYYAMDYWGQGTTVTVSS

SEQ ID NO: 9 - DNA, 363 bp, synthetic construct
  misc_feature 1..363: note = Synthetic: VH12 heavy chain variable region (DNA)
  source 1..363: mol_type = other DNA
caggtccagcttgtgcagtctggggctgaggtgaagaagcctggggcctcagtgaaggtctcctgcaaggcttctggatacacattcactagctatgtgatgcactgggtgcgacaggcccctggacaagggcttgagtggatgggatatattaatccttataatgatgctcctaaatacaatgagaagttcaaaggcagggtcaccatgaccagggacacgtccatcagcacagcctacatggagctgagcaggctgagatctgacgacacggccgtgtattactgtgcgagaggcttgggttacgccctttactatgctatggactactggggccaagggaccacggtcaccgtctcgagc

SEQ ID NO: 10 - protein, 121 aa, synthetic construct
  REGION 1..121: note = Synthetic: VH12 heavy chain variable region (AA)
QVQLVQSGAEVKKPGASVKVSCKASGYTFTSYVMHWVRQAPGQGLEWMGYINPYNDAPKYNEKFKGRVTMTRDTSISTAYMELSRLRSDDTAVYYCARGLGYALYYAMDYWGQGTTVTVSS

SEQ ID NO: 11 - DNA, 363 bp, synthetic construct
  misc_feature 1..363: note = Synthetic: VH13 heavy chain variable region (DNA)
  source 1..363: mol_type = other DNA
caggtccagcttgtgcagtctggggctgaggtgaagaagcctggggcctcagtgaaggtttcctgcaaggcatctggatacacattcactagctatgtgatgcactgggtgcgacaggcccctggacaagggcttgagtggatgggatatattaatccttataatgatgctcctaaatacaatgagaagttcaaaggcagagtcaccatgaccagggacacgtccacgagcacagtctacatggagctgagcagcctgagatctgaggacacggccgtgtattactgtgcgagaggcttgggttacgccctttactatgctatggactactggggccaagggacaatggtcaccgtctcgagc

SEQ ID NO: 12 - protein, 121 aa, synthetic construct
  REGION 1..121: note = Synthetic: VH13 heavy chain variable region (AA)
QVQLVQSGAEVKKPGASVKVSCKASGYTFTSYVMHWVRQAPGQGLEWMGYINPYNDAPKYNEKFKGRVTMTRDTSTSTVYMELSSLRSEDTAVYYCARGLGYALYYAMDYWGQGTMVTVSS

SEQ ID NO: 13 - DNA, 363 bp, synthetic construct
  misc_feature 1..363: note = Synthetic: VH14 heavy chain variable region (DNA)
  source 1..363: mol_type = other DNA
caggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcagagtgaccatgaccagagacaccagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 14 - protein, 121 aa, synthetic construct
  REGION 1..121: note = Synthetic: VH14 heavy chain variable region (AA)
QVQLVQSGAEVKKPGASVKVSCKASGYTFTSYVMHWVRQAPGQGLEWMGYINPYNDAPKYNEKFKGRVTMTRDTSASTAYMELSSLRSDDTAVYYCARGLGYALYYAMDYWGQGTTVTVSS

SEQ ID NO: 15 - DNA, 363 bp, synthetic construct
  misc_feature 1..363: note = Synthetic: VH14_1 heavy chain variable region (DNA)
  source 1..363: mol_type = other DNA
caggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcagagtgaccatgaccagtgacaccagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 16 - protein, 121 aa, synthetic construct
  REGION 1..121: note = Synthetic: VH14_1 heavy chain variable region (AA)
QVQLVQSGAEVKKPGASVKVSCKASGYTFTSYVMHWVRQAPGQGLEWMGYINPYNDAPKYNEKFKGRVTMTSDTSASTAYMELSSLRSDDTAVYYCARGLGYALYYAMDYWGQGTTVTVSS

SEQ ID NO: 17 - DNA, 363 bp, synthetic construct
  misc_feature 1..363: note = Synthetic: VH14_1C heavy chain variable region (DNA)
  source 1..363: mol_type = other DNA
caggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcagagtgaccatcaccagtgacaccagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 18 - protein, 121 aa, synthetic construct
  REGION 1..121: note = Synthetic: VH14_1C heavy chain variable region (AA)
QVQLVQSGAEVKKPGASVKVSCKASGYTFTSYVMHWVRQAPGQGLEWMGYINPYNDAPKYNEKFKGRVTITSDTSASTAYMELSSLRSDDTAVYYCARGLGYALYYAMDYWGQGTTVTVSS

SEQ ID NO: 19 - DNA, 363 bp, synthetic construct
  misc_feature 1..363: note = Synthetic: VH14_1D heavy chain variable region (DNA)
  source 1..363: mol_type = other DNA
caggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcagagtgaccatgaccagtgacaagagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 20 - protein, 121 aa, synthetic construct
  REGION 1..121: note = Synthetic: VH14_1D heavy chain variable region (AA)
QVQLVQSGAEVKKPGASVKVSCKASGYTFTSYVMHWVRQAPGQGLEWMGYINPYNDAPKYNEKFKGRVTMTSDKSASTAYMELSSLRSDDTAVYYCARGLGYALYYAMDYWGQGTTVTVSS

SEQ ID NO: 21 - DNA, 363 bp, synthetic construct
  misc_feature 1..363: note = Synthetic: VH14_1E heavy chain variable region (DNA)
  source 1..363: mol_type = other DNA
gaggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcagagtgaccatgaccagtgacaccagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 22 - protein, 121 aa, synthetic construct
  REGION 1..121: note = Synthetic: VH14_1E heavy chain variable region (AA)
EVQLVQSGAEVKKPGASVKVSCKASGYTFTSYVMHWVRQAPGQGLEWMGYINPYNDAPKYNEKFKGRVTMTSDTSASTAYMELSSLRSDDTAVYYCARGLGYALYYAMDYWGQGTTVTVSS

SEQ ID NO: 23 - DNA, 363 bp, synthetic construct
  misc_feature 1..363: note = Synthetic: VH14_1G heavy chain variable region (DNA)
  source 1..363: mol_type = other DNA
caggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcaaagcgaccatgaccagtgacaccagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 24 - protein, 121 aa, synthetic construct
  REGION 1..121: note = Synthetic: VH14_1G heavy chain variable region (AA)
QVQLVQSGAEVKKPGASVKVSCKASGYTFTSYVMHWVRQAPGQGLEWMGYINPYNDAPKYNEKFKGKATMTSDTSASTAYMELSSLRSDDTAVYYCARGLGYALYYAMDYWGQGTTVTVSS

SEQ ID NO: 25 - DNA, 321 bp, synthetic construct
  misc_feature 1..321: note = Synthetic: VL15 light chain variable region (DNA)
  source 1..321: mol_type = other DNA
gacatcgtgatgacccagtctccttccaccctgtctgcatctgtaggagacagagtcaccatcacttgcaaggccagtcagaatgtgggtaataatgtagcctggtatcagcagaaaccagggaaagcccctaagctcctgatctcttcggcatccaaccgtgacagtggggtcccatcaaggttcagcggcagtggatctgggacagagttcactctcaccatcagcagcctgcagcctgatgattttgcaacttatttctgccagcaatataacatctatccattcacgtttggccaggggaccaagctggagatcaaa

SEQ ID NO: 26 - protein, 107 aa, synthetic construct
  REGION 1..107: note = Synthetic: VL15 light chain variable region (AA)
DIVMTQSPSTLSASVGDRVTITCKASQNVGNNVAWYQQKPGKAPKLLISSASNRDSGVPSRFSGSGSGTEFTLTISSLQPDDFATYFCQQYNIYPFTFGQGTKLEIK

SEQ ID NO: 27 - DNA, 1344 bp, synthetic construct
  misc_feature 1..1344: note = Synthetic: VH14_1G heavy chain (DNA)
  source 1..1344: mol_type = other DNA
caggtccaacttgtgcagtctggggctgaagtgaagaagcccggcgctagtgtgaaggtttcatgtaaggcttctggatacacatttacttcatatgtaatgcactgggtgcgtcaagcccctggccagggcctggagtggatggggtatattaatccttataatgatgctcctaaatacaatgagaagttcaaaggaaaagcaactatgaccagtgatactagcgcttcaaccgcctacatggagctgagcagcttaagaagcgacgacaccgccgtgtactattgtgccaggggcttgggttacgccctttattatgctatggactactggggtcagggcaccacagtgaccgttagctctgcatctactaagggaccatccgtcttccccctggcgccatgctcccgcagtacaagtgagagcacagcagccctgggctgtttggtaaaggactacttccccgaacctgtgactgtgtcttggaactcaggcgccctgactagcggcgtgcacactttccctgctgtcctacagtcctcaggactatactccctctcgtctgtggtgacagtgccttcctcatcattgggaacgaaaacctatacttgcaacgttgatcacaagcccagcaacaccaaggtggacaagagagttgagtccaaatatggtcccccatgtccaccatgtccagcacctgagtttcttggcggaccaagtgttttcctgttccccccaaaacccaaggatactctcatgataagtcgcacccctgaagtcacttgcgtggtggtggacgttagccaggaagatcccgaagtccaattcaactggtacgtagatggcgtagaagtgcataatgcgaagacaaagccgagagaggagcagtttaattcgacgtatcgggtggtcagcgtcctcacagtcctgcaccaggactggctgaacggcaaggagtataagtgcaaggtctccaacaaaggtctcccgtcctccattgagaaaacaatctccaaagcaaaagggcagccccgagaaccacaagtgtacaccctgcccccatctcaggaggagatgaccaagaaccaggtcagtcttacctgcctggtcaaaggcttttatccctcagatatcgccgttgagtgggaaagcaatgggcagccggagaacaactacaagaccacgcctcccgttctggattctgacggatcgttctttttatacagcaggctaaccgtggacaagtctcggtggcaggaagggaatgtattttcttgcagtgtaatgcatgaggctctgcacaatcattacacacagaagtctctctccctgtctcttggtaaa

SEQ ID NO: 28 - protein, 448 aa, synthetic construct
  REGION 1..448: note = Synthetic: VH14_1G heavy chain (AA)
QVQLVQSGAEVKKPGASVKVSCKASGYTFTSYVMHWVRQAPGQGLEWMGYINPYNDAPKYNEKFKGKATMTSDTSASTAYMELSSLRSDDTAVYYCARGLGYALYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - DNA, 642 bp, synthetic construct
  misc_feature 1..642: note = Synthetic: VL15 light chain (DNA)
  source 1..642: mol_type = other DNA
gacattgtgatgacccagtctccttccactctctctgcatctgtgggagacagagtaaccatcacttgcaaggccagtcagaatgtgggtaataatgtagcctggtatcagcagaaaccagggaaagcccctaagctactgatctcttccgcatccaaccgggacagtggtgtgccctcaaggtttagcggcagtggatcagggacagagttcacattgaccatatccagcctgcagcctgatgattttgctacttatttctgccaacaatataacatttacccattcacgtttggccagggcaccaagctagagatcaaacggacggttgctgcaccctctgtctttatcttcccgccatctgatgaacagttgaagtccggaacagcctctgttgtgtgcctgctgaataacttttatccccgcgaggcgaaagttcagtggaaggtggataacgccctccaatcaggcaattcccaggagagtgtgacagagcaagattccaaggactcaacctacagcctcagcagtactttaactctgagcaaagcagactacgagaagcacaaagtctacgcttgcgaagtcacccatcagggccttagctcgcccgtcacaaagagctttaacaggggagaatgt

SEQ ID NO: 30 - protein, 214 aa, synthetic construct
  REGION 1..214: note = Synthetic: VL15 light chain (AA)
DIVMTQSPSTLSASVGDRVTITCKASQNVGNNVAWYQQKPGKAPKLLISSASNRDSGVPSRFSGSGSGTEFTLTISSLQPDDFATYFCQQYNIYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 57 bp, Homo sapiens
  misc_feature 1..57: note = Heavy chain secretion leader sequence (DNA)
  source 1..57: mol_type = genomic DNA
atggactggacctggaggatcctctttttggtggcagcagccacaggtgcccactcc

SEQ ID NO: 32 - protein, 19 aa, Homo sapiens
  REGION 1..19: note = misc_feature - Heavy chain secretion leader sequence (AA)
MDWTWRILFLVAAATGAHS

SEQ ID NO: 33 - DNA, 66 bp, Homo sapiens
  misc_feature 1..66: note = Light chain secretion leader sequence (DNA)
  source 1..66: mol_type = genomic DNA
atggacatgagagtcctcgctcagctcctggggctcctgctgctctgtttcccaggtgccagatgt

SEQ ID NO: 34 - protein, 22 aa, Homo sapiens
  REGION 1..22: note = misc_feature - Light chain secretion leader sequence (AA)
MDMRVLAQLLGLLLLCFPGARC

(19) United States
(12) Patent Application Publication
(10) Pub. No.: US 2026/0355310 A1
(43) Pub. Date: Nov. 12, 2026

(54) TREATMENT OF IGA NEPHROPATHY USING AN ENDOTHELIN RECEPTOR ANTAGONIST AND APRIL BINDING ANTIBODY

(71) Applicants: CHINOOK THERAPEUTICS, INC., Seattle, WA (US); ADURO BIOTECH HOLDINGS, EUROPE B.V., Rosmalen (NL)

(72) Inventor: Andrew James KING, Dublin, CA (US)

(21) Appl. No.: 19/123,511

(22) PCT Filed: Oct. 27, 2023
(86) PCT No.: PCT/US2023/078096

§ 371 Date: Apr. 23, 2025

Related U.S. Application Data
(60) Provisional application No. 63/420,305, filed on Oct. 28, 2022.
(60) Provisional application No. 63/420,504, filed on Oct. 28, 2022.

(51) Int. Cl. A61K 39/395; A61K 31/4025; A61K 39/00; A61K 45/06; A61P 13/12

(52) U.S. Cl. CPC A61K 39/3955; A61K 31/4025; A61K 45/06; A61P 13/12; A61K 2039/505; A61K 2039/545

(57) ABSTRACT

The present disclosure relates to the use of an endothelin receptor antagonist, or a pharmaceutically acceptable salt thereof, and isolated antibodies, including antigen-binding fragments thereof, which bind human APRIL for the treatment of IgA nephropathy.

### CROSS-REFERENCE TO RELATED APPLICATIONS

This application claims the benefit of the filing date of U.S. Provisional Application No. 63/420,305, filed Oct. 28, 2022, and U.S. Provisional Application No. 63/420,504, filed Oct. 28, 2022. The contents of each application are incorporated by reference herein in their entirety.

### SEQUENCE LISTING

This document includes a sequence listing in electronic format submitted to the United States Patent and Trademark Office via the electronic filing system. The XML file, which is incorporated-by-reference herein, is titled “sequence.XML.” was created on Oct. 27, 2022, and has a size of 50.2 bytes.

### FIELD

The present disclosure relates to the use of an endothelin receptor antagonist, or a pharmaceutically acceptable salt thereof, and isolated antibodies, including fragments thereof, which bind human APRIL for the treatment of IgA nephropathy.

### BACKGROUND

IgA nephropathy (IgAN) is the most common primary glomerulonephritis worldwide. Aberrant glycosylation of IgAQ1 results in increased serum levels of galactose-deficient IgAQ1 (Gd-IgAQ1) that are recognized by glycan-specific IgA and IgG autoantibodies. Aggregates of the immune complexes are formed in situ and/or deposited in the glomerular mesangium. This promotes proliferation of mesangial cells, increased synthesis of extracellular matrix proteins, cytokines, chemokines, and infiltration of immune cells into the surrounding tissue. Accordingly, disease progression involves (1) production of Gd-IgAQ1; and (2) its recognition by antiglycan autoantibodies; which (3) form immune complexes in the kidney; and (4) activate mesangial cells. See, e.g., Penfold et al., Int. J. Nephrol. and Renovascular Dis. 11, pp. 137-148 (2017).

Unlike other progressive kidney diseases such as diabetic nephropathy, IgAN occurs primarily in subjects in their 20s and 30s who are otherwise healthy. Patients present with a range of symptoms, typically including micro- or macro-hematuria and increased protein excretion in the urine. Patients may also present with hypertension as a result of sustained renal damage. Current therapeutic approaches merely provide supportive care, including administration of the maximum tolerable dose of an angiotensin converting enzyme inhibitor or angiotensin-receptor blocker, or administration of immunosuppressive drugs, whose benefits are largely outweighed by adverse reactions. Ultimately, 30-40% of patients will develop end-stage renal disease (ESRD) within 20-30 years of diagnosis of IgAN. In the interim, patients experience numerous symptoms that significantly degrade their quality of life, in addition to declining renal function. Patients with IgAN often exhibit significantly increased expressions of endothelin 1 (ET-1) and ET-RA in the kidney. Increased expression of endothelins positively correlates with proteinuria, one of the hallmark symptoms of IgAN.

APRIL is expressed as a type-II transmembrane protein, but unlike most other TNF family members it is mainly processed as a secreted protein and cleaved in the Golgi apparatus where it is cleaved by a furin convertase to release a soluble active form (Lopez-Fraga et al., 2001, EMBO Rep 2:945-51,). APRIL assembles as a non-covalently linked homo-trimer with similar structural homology in protein fold to a number of other TNF family ligands (Wallweber et al., 2004, Mol Biol 343, 283-90). APRIL binds two TNF receptors: B cell maturation antigen (BCMA) and transmembrane activator and calcium modulator and cyclophilin ligand interactor (TACI) (reviewed in Kimberley et al., 2009, J Cell Physiol. 218(1):1-8). In addition, APRIL has recently been shown to bind heparan sulphate proteoglycans (HSPGs) (Hendriks et al., 2005, Cell Death Differ 12, 637-48). APRIL has been shown to have a role in B cell signaling and drive both proliferation and survival of human and murine B cells in-vitro (reviewed in Kimberley et al., 2009. J Cell Physiol. 218(1):1-8).

APRIL is predominantly expressed by immune cell subsets such as monocytes, macrophages, dendritic cells, neutrophils, B-cells, and T-cells, many of which also express BAFF. In addition, APRIL can be expressed by non-immune cells such as osteoclasts, epithelial cells and a variety of tumour tissues (reviewed in Kimberlev et al., 2009, J Cell Physiol. 218(1):1-8). In fact, APRIL was originally identified based on its expression in cancer cells (Hahne et al., 1998, J Exp Med 188, 1185-90). High expression levels of APRIL mRNA were found in a panel of tumour cell lines as well as human primary tumours such as colon, and a lymphoid carcinoma. APRIL serum levels were found to be increased in patients suffering from IgA nephropathy (McCarthy et al., 2011, J. Clin. Invest. 121(10):3991-4002).

Serum Gd-IgAQ1 levels are reportedly significantly higher in IgAN patients than disease controls and healthy controls. In patients with IgAN, serum Gd-IA1 levels were significantly correlated with estimated glomerular filtration rate, serum IgA level, and tubular atrophy/interstitial fibrosis. CKD progression was more frequent in IgAN patients with higher serum Gd-IgAQ1 levels than in those with lower serum Gd-IgA1 levels. Cox proportional hazard models showed that high GdIgA1 level was an independent risk factor for CKD progression after adjusting for several confounders. Kim et al., J. Clin. Med. 2020 Nov. 4; 9(11):3549. doi: 10.3390/jcm9113549.

The established standard of care for most patients with IgA nephropathy involves providing supportive measures that include the use of renin-angiotensin-aldosterone system blockade. Many patients are also offered corticosteroid therapy, or other immunosuppressive agents, despite clear evidence that the benefits of these therapies outweigh the risks.

In an effort to improve care for IgAN patients, at least two antibodies directed against APRIL, BION-1301 and sibeprenlimab (VIS649) are in clinical trials for the treatment of IgAN. See, e.g., US ClinicalTrials.gov identifiers NCT05508204, NCT03945318, NCT03719443, NCT05248659, and NCT05248646. Similarly, the endothelin receptor antagonists Atrasentan and Sparsentan are in clinical trial for the treatment of IgAN. See, e.g., US ClinicalTrials.gov identifiers NCT04573478, NCT05834738, NCT04573920 and NCT04663204. Additionally, a subanalysis of the Dapagliflozin and Prevention of Adverse Outcomes in Chronic Kidney Disease (DAPA-CKD) trial provided early evidence that dapagliflozin, an SGLT-2 inhibitor, may be a safe and effective addition to current standard of care in IgA nephropathy. Wheeler et al., Kidney International (2021) 100: 215-224.

### SUMMARY

Atrasentan is a selective endothelin A (ETA) receptor antagonist (ETA Ki ~34pM; ETB Ki~63 nM, ETA selectivity ~1800x). See, e.g., Wu-Wong et al., Clin. Sci. (Lond.), 103(48), pp. 107s-111s (2002). Selective ETA receptor antagonists block ETA function, while minimally effecting the ETB receptor, providing beneficial renal effects including vasodilation and reduction of inflammation, while still enabling ET-1 clearance. See e.g., Jandeleit-Dahm and Watson, Curr. Opin. Nephrol. Hypertens., 21(1), pp. 66-71 (2012); see also, Nakamura, et al., Nephron, Vol. 72, pp. 454-460 (1996). While ETA receptor antagonists increase sodium and water retention by the kidney, this is typically clinically manageable. See, e.g., Saleh, et al., J. Pharm. Exp. Ther., 338(1). pp. 263-270 (2011). Atrasentan has been shown to be effective in patients with diabetic kidney disease (DKD), significantly reducing the risk of renal events defined as a doubling of serum creatinine or end-stage kidney disease. See, e.g., Heerspink, et al., The Lancet, 393, pp 1937-1947 (2019).

Sparsentan is a dual-acting angiotensin II subtype 1 receptor blocker (“ARB”) and an ETA receptor antagonist. Trachtman, et al., J. Am. Soc. Nephrol. Vol. 29, No. 11, pp. 2745-2754 (2018).

IgA nephropathy is considered a primary glomerular disease where localized or intrinsic renal pathology is present. The peak incidence of IgA nephropathy is in young individuals in their second or third decade of life and is a disease that is auto-immune in origin, unlike, for example, diabetic kidney disease (“DKD”). IgA nephropathy results from pathogenic IgA/immune complex deposition in the glomerular mesangium. See, e.g., Lai, et al., Nature Reviews Disease Primers, 2, pp. 16001, 2016. Definitive diagnosis requires kidney biopsy and demonstration of mesangial IgA deposition by immunofluorescence microscopy. Recent advances in the understanding of the initiating events triggering IgA nephropathy have revealed that an aberrant mucosal immune response stimulates the production of galactose deficient IgAQ1 which is recognized as an autoantigen by circulating antiglycan autoantibodies. Immune recognition results in the formation of nephritogenic immune complexes that deposit in the kidney and activate mesangial cells. Activated mesangial cells proliferate and produce excess amounts of extracellular matrix components, cytokines and chemokines. See, e.g., Suzuki, et al., J. Am. Soc. Nephrol., Vol. 22, pp. 1795-1803 (2011). Up to 40% of patients with biopsy-proven IgA nephropathy progress to end stage kidney disease at some point during long term follow up. As further described herein, atrasentan can be administered at an efficacious dose with acceptable toxicity, and has the appropriate selectivity to minimize undesired side effects while still treating the underlying IgAN and improving subjects' quality of life. Some embodiments provide a method of inhibiting mesangial cell activation in a subject having IgA nephropathy, comprising administering an endothelin receptor antagonist, or a pharmaceutically acceptable salt thereof, and an APRIL binding antibody or an antigen-binding fragment thereof, to the subject.

Most subjects with IgAN first present with either single or episodic macroscopic hematuria, or after detection of microscopic hematuria and/or proteinuria during routine urine testing. In some cases, subjects present with acute kidney injury, such as a result of crescentic IgAN or gross hematuria causing tubular obstruction. Definitive diagnosis of IgAN is typically established by kidney biopsy, with immunofluorescence and/or immunoperoxidase studies for IgA deposits. Prominent, globular deposits of IgA (sometimes accompanied by C3 and IgG) in the mesangium and less prominently along the glomerular capillary wall are a hallmark of IgAN. Certain histopathological features that correlate with long-term outcome include mesangial proliferation, endocapillary proliferation, segmental scarring, and tubular atrophy.

Some embodiments provide a method of treating IgA nephropathy, comprising administering to the subject an endothelin receptor antagonist, or a pharmaceutically acceptable salt thereof, and an APRIL binding antibody or an antigen-binding fragment thereof.

Some embodiments provide a method of inhibiting mesangial cell activation in a subject having IgA nephropathy, comprising administering to the subject an endothelin receptor antagonist, or a pharmaceutically acceptable salt thereof, and an APRIL binding antibody or an antigen-binding fragment thereof.

In some embodiments, the inhibiting of mesangial cell activation comprises reducing mesangial cell inflammation and/or activity of one or more biomarkers indicative of mesangial cell proliferation.

In some embodiments, reducing mesangial cell inflammation comprises reducing expression and/or activity of one or more of IL6, MCP1 or other biomarkers indicative of mesangial cell inflammation.

In some embodiments, the inhibiting of mesangial cell activation comprises reducing the pro-fibrotic response in the mesangial cells.

In some embodiments, the mesangial activation is induced by IgA immune complexes.

In some embodiments, the mesangial activation is associated with the presence of IgA immune complexes.

Some embodiments provide a method of decreasing renal inflammation and/or fibrosis in a subject having IgA nephropathy, comprising administering to the subject an endothelin receptor antagonist, or a pharmaceutically acceptable salt thereof, and an APRIL binding antibody or an antigen-binding fragment thereof.

Some embodiments provide a method of decreasing the occurrence of hematuria in a subject having IgA nephropathy, comprising administering to the subject an endothelin receptor antagonist, or a pharmaceutically acceptable salt thereof, and an APRIL binding antibody or an antigen-binding fragment thereof.

Some embodiments provide a method of stabilizing eGFR in a subject having IgA nephropathy, comprising administering to the subject an endothelin receptor antagonist, or a pharmaceutically acceptable salt thereof, and an APRIL binding antibody or an antigen-binding fragment thereof.

Some embodiments provide a method of decreasing the number of IgA-nephropathy associated disease flares in a subject having IgA nephropathy, comprising administering to the subject an endothelin receptor antagonist, or a pharmaceutically acceptable salt thereof, and an APRIL binding antibody or an antigen-binding fragment thereof.

Some embodiments provide a method of delaying the onset of end-stage renal disease (ESRD) in a subject having IgA nephropathy, comprising administering to the subject an endothelin receptor antagonist, or a pharmaceutically acceptable salt thereof, and an APRIL binding antibody or an antigen-binding fragment thereof.

Some embodiments provide a method of decreasing proteinuria in a subject having IgA nephropathy comprising administering to the subject an endothelin receptor antagonist, or a pharmaceutically acceptable salt thereof, and an APRIL binding antibody or an antigen-binding fragment thereof.

Some embodiments provide a method of decreasing fatigue in a subject having IgA nephropathy, comprising administering to the subject an endothelin receptor antagonist, or a pharmaceutically acceptable salt thereof, and an APRIL binding antibody or an antigen-binding fragment thereof.

In some embodiments, the step of determining that the subject has IgA-immune complex deposition in the kidney occurs prior to the administering step. In some embodiments, the step of determining that the subject has elevated levels of mesangial activation occurs prior to the administering step. In some embodiments, the step of determining that the subject has elevated levels of IgA-immune complexes in the kidney occurs prior to the administering step.

In some embodiments, the endothelin receptor antagonist, or a pharmaceutically acceptable salt thereof, is selected from the group consisting of tezosentan, spysentan, bosentan, sparsentan, macitentan, ambrisentan, sitaxentan, atriopeptine, atrasentan, and pharmaceutically acceptable salts of any of the foregoing, and combinations thereof.

In some embodiments, the endothelin receptor antagonist, or a pharmaceutically acceptable salt thereof, is sparsentan, or a pharmaceutically acceptable salt thereof, and the APRIL binding antibody or antigen-binding fragment thereof.

In some embodiments, the endothelin receptor antagonist, or a pharmaceutically acceptable salt thereof, is atrasentan, or a pharmaceutically acceptable salt thereof. In some embodiments, the atrasentan is administered as a pharmaceutically acceptable salt. In some embodiments, the pharmaceutically acceptable salt of atrasentan is atrasentan hydrochloride or atrasentan mandelate. In some embodiments, the pharmaceutically acceptable salt of atrasentan is atrasentan hydrochloride. In some embodiments, the pharmaceutically acceptable salt of atrasentan is atrasentan mandelate. In some embodiments, the atrasentan is administered as the free base.

In some embodiments, the APRIL binding antibody or antigen-binding fragment thereof comprises:
a heavy chain complementarity-determining region-1 (HC CDR1) comprising an amino acid sequence having at least 80% sequence identity to SEQ ID NO:1;
a heavy chain complementarity-determining region-2 (HC CDR2) comprising an amino acid sequence having at least 80% sequence identity to SEQ ID NO:2;
a heavy chain complementarity-determining region-3 (HC CDR3) comprising an amino acid sequence having at least 80% sequence identity to SEQ ID NO:3;
a light chain complementarity-determining region-1 (LC CDR1) comprising an amino acid sequence having at least 80% sequence identity to SEQ ID NO:4;
a light chain complementarity-determining region-2 (LC CDR2) comprising an amino acid sequence having at least 80% sequence identity to SEQ ID NO:5; and a light chain complementarity-determining region-3 (LC CDR3) comprising an amino acid sequence having at least 80% sequence identity to SEQ ID NO:6.

In some embodiments, the APRIL binding antibody or antigen-binding fragment thereof comprises:
a heavy chain complementarity-determining region-1 (HC CDR1) comprising an amino acid sequence having at least 90% sequence identity to SEQ ID NO:1;
a heavy chain complementarity-determining region-2 (HC CDR2) comprising an amino acid sequence having at least 90% sequence identity to SEQ ID NO:2;
a heavy chain complementarity-determining region-3 (HC CDR3) comprising an amino acid sequence having at least 90% sequence identity to SEQ ID NO:3;
a light chain complementarity-determining region-1 (LC CDR1) comprising an amino acid sequence having at least 90% sequence identity to SEQ ID NO:4;
a light chain complementarity-determining region-2 (LC CDR2) comprising an amino acid sequence having at least 90% sequence identity to SEQ ID NO:5; and a light chain complementarity-determining region-3 (LC CDR3) comprising an amino acid sequence having at least 90% sequence identity to SEQ ID NO:6.

In some embodiments, the APRIL binding antibody or antigen-binding fragment thereof comprises:
a heavy chain complementarity-determining region-1 (HC CDR1) comprising an amino acid sequence of SEQ ID NO: 1;
a heavy chain complementarity-determining region-2 (HC CDR2) comprising an amino acid sequence of SEQ ID NO:2;
a heavy chain complementarity-determining region-3 (HC CDR3) comprising an amino acid sequence of SEQ ID NO:3;
a light chain complementarity-determining region-1 (LC CDR1) comprising an amino acid sequence of SEQ ID NO:4;
a light chain complementarity-determining region-2 (LC CDR2) comprising an amino acid sequence of SEQ ID NO:5; and
a light chain complementarity-determining region-3 (LC CDR3) comprising an amino acid sequence of SEQ ID NO:6.

In some embodiments, the APRIL binding antibody or antigen-binding fragment thereof comprises a heavy chain variable region comprising an amino acid sequence having at least 80% sequence identity to SEQ ID NOs: 8, 10, 12, 14, 16, 18, 20, 22, or 24.

In some embodiments, the APRIL binding antibody or antigen-binding fragment thereof comprises a heavy chain variable region comprising an amino acid sequence having at least 90% sequence identity to SEQ ID NOs: 8, 10, 12, 14, 16, 18, 20, 22, or 24.

In some embodiments, the APRIL binding antibody or antigen-binding fragment thereof comprises a heavy chain variable region comprising an amino acid sequence of SEQ ID NOs: 8, 10, 12, 14, 16, 18, 20, 22, or 24.

In some embodiments, the APRIL binding antibody or antigen-binding fragment thereof comprises a light chain variable region comprising an amino acid sequence having at least 80% sequence identity to SEQ ID NO: 26

In some embodiments, the APRIL binding antibody or antigen-binding fragment thereof comprises a light chain variable region comprising an amino acid sequence having at least 90% sequence identity to SEQ ID NO: 26.

In some embodiments, the APRIL binding antibody or antigen-binding fragment thereof comprises a light chain variable region comprising an amino acid sequence of SEQ ID NO: 26.

In some embodiments, the APRIL binding antibody or antigen-binding fragment thereof comprises a heavy chain comprising an amino acid sequence having at least 80% sequence identity to SEQ ID NO: 28.

In some embodiments, the APRIL binding antibody or antigen-binding fragment thereof comprises a heavy chain comprising an amino acid sequence having at least 90% sequence identity to SEQ ID NO: 28.

In some embodiments, the APRIL binding antibody or antigen-binding fragment thereof comprises a heavy chain comprising an amino acid sequence of SEQ ID NO: 28.

In some embodiments, the APRIL binding antibody or antigen-binding fragment thereof comprises a light chain comprising an amino acid sequence having at least 80% sequence identity to SEQ ID NO: 30.

In some embodiments, the APRIL binding antibody or antigen-binding fragment thereof comprises a light chain comprising an amino acid sequence having at least 90% sequence identity to SEQ ID NO: 30.

In some embodiments, the APRIL binding antibody or antigen-binding fragment thereof comprises a light chain comprising an amino acid sequence of SEQ ID NO: 30.

In some embodiments, the APRIL binding antibody or antigen-binding fragment thereof comprises a heavy chain secretion leader comprising an amino acid sequence having at least 80% sequence identity to SEQ ID NO: 32.

In some embodiments, the APRIL binding antibody or antigen-binding fragment thereof comprises a heavy chain secretion leader comprising an amino acid sequence having at least 90% sequence identity to SEQ ID NO: 32.

In some embodiments, the APRIL binding antibody or antigen-binding fragment thereof comprises a heavy chain secretion leader comprising an amino acid sequence of SEQ ID NO: 32.

In some embodiments, the APRIL binding antibody or antigen-binding fragment thereof comprises a light chain secretion leader comprising an amino acid sequence having at least 80% sequence identity to SEQ ID NO: 34.

In some embodiments, the APRIL binding antibody or antigen-binding fragment thereof comprises a light chain secretion leader comprising an amino acid sequence having at least 90% sequence identity to SEQ ID NO: 34.

In some embodiments, the APRIL binding antibody or antigen-binding fragment thereof comprises a light chain secretion leader comprising an amino acid sequence of SEQ ID NO: 34.

In some embodiments, the APRIL binding antibody or antigen-binding fragment thereof is generated from a nucleic acid comprising one of SEQ ID NOs: 7, 9, 11, 13, 15, 17, 19, 21, or 23.

In some embodiments, the APRIL binding antibody or antigen-binding fragment thereof is generated from a nucleic acid comprising SEQ ID NO: 25.

In some embodiments, the APRIL binding antibody or antigen-binding fragment thereof is generated from a nucleic acid comprising SEQ ID NO: 27.

In some embodiments, the APRIL binding antibody or antigen-binding fragment thereof is generated from a nucleic acid comprising SEQ ID NO: 29.

In some embodiments, the APRIL binding antibody or antigen-binding fragment thereof is generated from a nucleic acid comprising SEQ ID NO: 31.

In some embodiments, the APRIL binding antibody or antigen-binding fragment thereof is generated from a nucleic acid comprising SEQ ID NO: 33.

In some embodiments, the method comprises administering to the subject an amount of atrasentan, or a pharmaceutically acceptable salt thereof, equivalent to about 0.20 mg to about 1.50 mg of atrasentan free base. In some embodiments, the method comprises administering to the subject an amount of atrasentan hydrochloride equivalent to about 0.20 mg to about 1.50 mg of atrasentan free base.

In some embodiments, the method comprises administering to the subject an amount of atrasentan, or a pharmaceutically acceptable salt thereof, equivalent to about 0.25 mg to about 1.25 mg of atrasentan free base. In some embodiments, the method comprises administering to the subject an amount of atrasentan hydrochloride equivalent to about 0.25 mg to about 1.25 mg of atrasentan free base.

In some embodiments, the method comprises administering to the subject an amount of an endothelin receptor antagonist, or a pharmaceutically acceptable salt thereof, equivalent to about 0.40 mg to about 0.85 mg of atrasentan free base. In some embodiments, the method comprises administering to the subject an amount of atrasentan hydrochloride equivalent to about 0.40 mg to about 0.85 mg of atrasentan free base.

In some embodiments, the method comprises administering to the subject an amount of atrasentan, or a pharmaceutically acceptable salt thereof, equivalent to about 0.50 mg of atrasentan free base. In some embodiments, the method comprises administering to the subject an amount of atrasentan hydrochloride equivalent to about 0.50 mg of atrasentan free base.

In some embodiments, the method comprises administering to the subject an amount of atrasentan, or a pharmaceutically acceptable salt thereof, equivalent to about 0.75 mg of atrasentan free base. In some embodiments, the method comprises administering to the subject an amount of atrasentan hydrochloride equivalent to about 0.75 mg of atrasentan free base.

In some embodiments, the method comprises administering to the subject an amount of sparsentan, or a pharmaceutically acceptable salt thereof, equivalent to about 200 mg of sparsentan free base.

In some embodiments, the method comprises administering to the subject an amount of sparsentan, or a pharmaceutically acceptable salt thereof, equivalent to about 400 mg of sparsentan free base.

In some embodiments, the method comprises administering to the subject an amount of sparsentan, or a pharmaceutically acceptable salt thereof, equivalent to about 600 mg of sparsentan free base.

In some embodiments, the method comprises administering to the subject an amount of sparsentan, or a pharmaceutically acceptable salt thereof, equivalent to about 800 mg of sparsentan free base.

In some embodiments, the endothelin receptor antagonist, or a pharmaceutically acceptable salt thereof, is administered orally, buccally or parenterally, for example, the dosage form of the oral route may be tablets, capsules, powders, pills, granules, suspensions, solutions and solution preconcentrates, emulsions and emulsion preconcentrates, and for example, the dosage form of the parenteral route may be intravenous, intraperitoneal, intradermal, subcutaneous, intramuscular, intracranial, intrathecal, transdermal, transmucosal administration.

In some embodiments, the endothelin receptor antagonist, or a pharmaceutically acceptable salt thereof, is administered once per day.

In some embodiments, the APRIL binding antibody or antigen-binding fragment thereof is administered by a parenteral route, such as intravenous, intraperitoneal, intradermal, subcutaneous, muscle, intracranial, intrathecal, transdermal penetration, transmucosal administration.

In some embodiments, the APRIL binding antibody or antigen-binding fragment thereof is formulated into a solution, lyophilized agent, or a powder for injection.

In some embodiments, the APRIL binding antibody or antigen-binding fragment thereof is administered once every 2, 3, 4, 5, 6, 7, 8, 9 or 10 days or every 1, 2 or 3 weeks; alternatively, the APRIL binding antibody or antigen-binding fragment thereof may be administered once a day for five consecutive days per week, followed by an interval of two days.

In some embodiments, about 0.05 mg/kg to about 8 mg/kg of the APRIL binding antibody or antigen-binding fragment thereof is administered to the subject.

In some embodiments, about 0.05 mg/kg to about 4 mg/kg of the APRIL binding antibody or antigen-binding fragment thereof is administered to the subject.

In some embodiments, about 2 mg/kg to about 6 mg/kg of the APRIL binding antibody or antigen-binding fragment thereof is administered to the subject.

In some embodiments, about 4 mg/kg to about 8 mg/kg of the APRIL binding antibody or antigen-binding fragment thereof is administered to the subject.

In some embodiments, about 0.05 mg/kg, about 0.1 mg/kg, about 1 mg/kg, about 2 mg/kg, about 3 mg/kg, about 4 mg/kg, about 5 mg/kg, about 6 mg/kg, about 7 mg/kg, or about 8 mg/kg of the APRIL binding antibody or antigen-binding fragment thereof is administered to the subject.

In some embodiments, the method comprises repeating the administration of the APRIL binding antibody or antigen-binding fragment thereof on at least an every week (QW) schedule for at least 2 administration cycles.

In some embodiments, the method comprises repeating the administration of the APRIL binding antibody or antigen-binding fragment thereof on at least an every two weeks (Q2W) schedule for at least 2 administration cycles.

In some embodiments, the method comprises repeating the administration of the APRIL binding antibody or antigen-binding fragment thereof on at least an every 4 weeks (Q4W) or monthly (QMT) schedule for at least 2 administration cycles.

In some embodiments, a total dose of the APRIL binding antibody or antigen-binding fragment thereof of between about 10 mg to about 1350 mg of the APRIL binding antibody or antigen-binding fragment thereof is administered per dosing event.

In some embodiments, about 2 mL of the formulation at a concentration of about 150 mg/mL of the APRIL binding antibody or antigen-binding fragment thereof is delivered per administration, and each dosing event comprises one or more of said administrations.

In some embodiments, about 4 mL of the formulation at a concentration of about 150 mg/mL of the APRIL binding antibody or antigen-binding fragment thereof is delivered per administration, and each dosing event comprises one or more of said administrations.

In some embodiments, the formulation of the APRIL binding antibody or antigen-binding fragment thereof is subcutaneously administered into a site in the individual's thigh, abdomen, or upper arm.

In some embodiments, the formulation of the APRIL binding antibody or antigen-binding fragment thereof is intravenously administered.

In some embodiments, 15 mL of the formulation at a concentration of 20 mg/mL is added to 235 mL of 0.9% saline to provide the intravenous dose at a concentration of 1.2 mg/mL.

In some embodiments, administering the APRIL binding antibody or antigen-binding fragment thereof by a loading/maintenance administration protocol. In some embodiments, the loading component of the loading/maintenance administration protocol comprises one or more administrations of the APRIL binding antibody or antigen-binding fragment thereof at a higher concentration than an APRIL binding antibody or antigen-binding fragment thereof concentration in the maintenance component of the loading/maintenance administration protocol. In some embodiments, the loading component of the loading/maintenance administration protocol comprises one or more administrations of the APRIL binding antibody or antigen-binding fragment thereof at a higher frequency than the frequency of administration of an APRIL binding antibody or antigen-binding fragment thereof in the maintenance component of the loading/maintenance administration protocol.

In some embodiments, the subject has been determined to have controlled serum glucose levels.

In some embodiments, the subject is concomitantly receiving an angiotensin converting enzyme (ACE) inhibitor, an angiotensin II receptor blockers (ARB), or a combination thereof.

In some embodiments, the ACE inhibitor is selected from the group consisting of quinapril, fosinopril, perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril.

In some embodiments, the ACE inhibitor is selected from the group consisting of quinapril, fosinopril, captopril, enalapril, and lisinopril.

In some embodiments, the ARB is selected from the group consisting of candesartan, candesartan cilexetil, eprosartan, irbesartan, losartan, olmesartan, olmesartan medoxomil, telmisartan, valsartan, azilsartan medoxomil, and BRA-657.

In some embodiments, the ARB is selected from the group consisting of candesartan, losartan, olmesartan, and valsartan.

In some embodiments, the method further comprises administering a therapeutically effective amount of a SGLT-2 inhibitor.

In some embodiments, the SGLT-2 inhibitor is selected from the group consisting of dapagliflozin, canagliflozin, ipragliflozin, empaglifozin, bexagliflozin, licogliflozin, janagliflozin (XZP-5695), tofogliflozin, ertugliflozin, henagliflozin (SHR-3824), enavogliflozin (DWP-16001), TA-1887 (3-(4-cyclopropylbenzyl)-4-fluoro-1-(P-D-glucopyranosyl)-1H-indole), indole-N-glycoside 18 (3-(4-ethylbenzyl)-1-(P-D-glucopyranosyl)-1H-indole), sotagliflozin, luseogliflozin, sergliflozin etabonate, remogliflozin, remogliflozin etabonate, and T-1095 (((2R,3S,4S,5R,6S)-6-(2-(3-(benzofuran-5-yl)propanoyl)-3-hydroxy-5-methylphenoxy)-3,4,5-trihydroxytetrahydro-2H-pyran-2-yl) etabonate).

In some embodiments, the SGLT-2 inhibitor is selected from the group consisting of bexagliflozin, canagliflozin, dapagliflozin, empagliflozin, ertugliflozin, ipragliflozin, luseogliflozin, remogliflozin, serfliflozin, licofliglozin, sotagliflozin, and tofogliflozin.

In some embodiments, the SGLT-2 inhibitor is canagliflozin, dapagliflozin, empagliflozin, or ertugliflozin.

In some embodiments, administering the combination comprises administering the APRIL binding antibody or antigen-binding fragment thereof and one or both of the endothelin receptor antagonist and SGLT-2 inhibitor simultaneously, separately, or sequentially.

In some embodiments, administering the combination comprises administering the APRIL binding antibody or antigen-binding fragment thereof and the endothelin receptor antagonist by different administration routes.

In some embodiments, the APRIL binding antibody or antigen-binding fragment thereof is administered intravenously or subcutaneously, and the endothelin receptor antagonist is administered orally.

In some embodiments, APRIL binding antibody or an antigen-binding fragment thereof and the endothelin receptor antagonist is administered on different dosing schedules.

In some embodiments, the subject is administered a SGLT-2 inhibitor and one or more ACE inhibitors and/or one or more ARBs.

In some embodiments, the subject is administered a SGLT-2 inhibitor and one or more ACE inhibitors.

In some embodiments, the subject is administered a SGLT-2 inhibitor and an ACE inhibitor.

In some embodiments, the subject is administered a SGLT-2 inhibitor and one or more ARBs.

In some embodiments, the subject is administered a SGLT-2 inhibitor and an ARB.

In some embodiments, the SGLT-2 inhibitor is administered orally, buccally or parenterally. In some embodiments, the dosage form of the oral route is selected from the group consisting of tablets, capsules, powders, pills, granules, suspensions, solutions and solution preconcentrates, emulsions and emulsion preconcentrates. In some embodiments, the parenteral route is selected from the group consisting of intravenous, intraperitoneal, intradermal, subcutaneous, intramuscular, intracranial, intrathecal, transdermal, transmucosal administration.

In some embodiments, the subject is excreting an average of about 0.5 grams or more of protein in the urine per day prior to the first administration of atrasentan or a pharmaceutically acceptable salt thereof.

In some embodiments, the subject is excreting an average of about 1 gram or more of protein in the urine per day prior to the first administration of atrasentan or a pharmaceutically acceptable salt thereof.

In some embodiments, the subject has an average eGFR of at least about 30 mL/min/1.73 m² prior to the first administration of atrasentan or a pharmaceutically acceptable salt thereof.

Some embodiments provide a kit comprising: endothelin receptor antagonist, or a pharmaceutically acceptable salt thereof, and an APRIL binding antibody or an antigen-binding fragment thereof, wherein the endothelin receptor antagonist, or a pharmaceutically acceptable salt thereof, and the APRIL binding antibody or antigen-binding fragment thereof can be in the same and/or separate dosage form. In some embodiments, the kit can further comprise an SGLT-2 inhibitor.

Some embodiments provide a kit comprising: an SGLT-2 inhibitor and an APRIL binding antibody or an antigen-binding fragment thereof, wherein the SGLT-2 inhibitor and the APRIL binding antibody or antigen-binding fragment thereof can be in the same and/or separate dosage form.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE FIGURES

FIG. 1 shows the study schema for the Phase 3, Randomized, Double-blind, Placebo-controlled Study of BION-1301 in Adults with IgA Nephropathy.

### DETAILED DESCRIPTION

### A. DEFINITIONS

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2^{nd} ed., 2002. CRC Press; The Dictionary of Cell and Molecular Biology, 3^{rd}d ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “about” when referring to a number or a numerical range means that the number or numerical range referred to is an approximation, for example, within experimental variability and/or statistical experimental error, and thus the number or numerical range may vary up to ±10% of the stated number or numerical range.

“Treatment” or “therapy” of a subject refers to any type of intervention or process performed on, or the administration of an active agent to, the subject with the objective of reversing, alleviating, ameliorating, inhibiting, or slowing down, the onset, progression, development, severity, or recurrence of a symptom, complication, condition, or biochemical indicia associated with a disease.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

The phrase “effective amount” or “therapeutically effective amount” means an amount of compound that, when administered to a subject in need of such treatment, is sufficient to (i) treat the indicated disease or disorder, (ii) attenuate, ameliorate, or eliminate one or more symptoms of the particular disease, condition, or disorder, or (iii) delay the onset of one or more symptoms of the particular disease, condition, or disorder described herein. When used in reference to treatment with more than one therapeutic agent, each agent can independently be administered in a therapeutically effective amount (e.g., an amount that would be therapeutically effective as a monotherapy) or the one or more therapeutic agents can together be a therapeutically effective amount (e.g., a therapeutically effective amount of a combination therapy) for treating the indicated disease or disorder. In other words, the amount of the individual components in a therapeutically effective amount of a combination therapy can, independently, be administered (in the combination) at less than a therapeutically effective amount when administered as a monotherapy. An effective amount of therapeutic will decrease the symptoms typically by at least 10%; usually by at least 20%; preferably at least about 30%; more preferably at least 40%, and most preferably by at least 50%.

The term “synergy” or “synergistic” is used herein to mean that the effect of the combination of the two therapeutic agents of the combination therapy described herein is greater than the sum of the effect of each agent when administered alone (i.e., as a monotherapy).

Determining a synergistic interaction between two combination partners, the optimum range for the effect and absolute dose ranges of each component for the effect may be definitively measured by administration of the combination partners over different w/w (weight per weight) ratio ranges and doses to subjects in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods. Exemplary synergistic effects includes, but are not limited to, enhanced therapeutic efficacy, decreased dosage at equal or increased level of efficacy, reduced or delayed development of drug resistance, and simultaneous enhancement or equal therapeutic actions (e.g., the same therapeutic effect as at least one of the therapeutic agents) and reduction of unwanted drug effects (e.g., side effects and adverse events) of at least one of the therapeutic agents.

As used herein, “inhibit” includes a postponement of development of the symptoms associated with disease and/or a reduction in the severity of such symptoms that will or are expected to develop with said disease. The terms further include ameliorating existing symptoms, preventing additional symptoms, and ameliorating or preventing the underlying causes of such symptoms. Thus, the terms denote that a beneficial result has been conferred on a vertebrate subject with a disease.

In some embodiments, the endothelin receptor antagonist, or a pharmaceutically acceptable salt thereof, exhibits synergy when administered with an APRIL binding antibody or an antigen-binding fragment thereof, as described herein. In some embodiments, the APRIL binding antibody or antigen-binding fragment thereof exhibits synergy when administered with an endothelin receptor antagonist, or a pharmaceutically acceptable salt thereof, as described herein.

The term “endothelin receptor antagonists” as used herein refers to compounds that inhibit or block the binding of endothelin with endothelin receptors. Endothelin (ET) is a highly potent vasoconstrictor peptide synthesized and released by the vascular endothelium. Endothelin exists as three isoforms, ET-1, ET-2 and ET-3, of which only ET-1 and ET-3 have been found to be expressed in mammalian systems (unless otherwise stated, “endothelin” as used herein shall mean any or all of the isoforms of endothelin). There are at least two major known endothelin receptors, ETA and ETB, both of which are G protein-coupled receptors that when activated result in elevation of intracellular-free calcium (Davenport (2002) Pharmacol. Rev. 54(2): 219-26). Selective ETA receptor antagonists include sitaxentan (CAS No. 184036-34-8, and as described in Barst et al. (2004) American J. Resp. Crit. Care Med. 169(4):441-7); ambrisentan (CAS No. 177036-94-1, and as described in U.S. Pat. Nos. 5,703,017, 5,932,730, and 7,109,205): atrasentan (CAS No. 173937-91-2, and as disclosed in U.S. Pat. No. 5,767,144); BQ-123 (CAS No. 136553-81-6), and zibotentan (CAS No. 186497-07-4). Dual antagonists of ETA and ETB include bosentan (CAS No. 147536-97-8, and as described in Bien et al. (2007) Cancer Res. 67(21):10428-35), macitentan (CAS No. 441798-33-0), and tezosentan (CAS No. 180384-57-0).

Atrasentan is a selective endothelin A (ETA) receptor antagonist (ETA Ki~34pM; ETB Ki~63 nM, ETA selectivity ~1800x). See, e.g. Wu-Wong et al., Clin. Sci. (Lond.), 103(48), pp. 107s-11 Is (2002). Selective ETA receptor antagonists block ETA function, while minimally effecting the ETB receptor, providing beneficial renal effects including vasodilation and reduction of inflammation, while still enabling ET-1 clearance. See e.g., Jandeleit-Dahm and Watson, Curr. Opin. Nephrol. Hypertens., 21(1), pp. 66-71 (2012); see also, Nakamura, et al., Nephron, Vol. 72, pp. 454-460 (1996). While ETA receptor antagonists increase sodium and water retention by the kidney, this is typically clinically manageable. See, e.g., Saleh, et al., J. Pharm, Exp. Then, 338(1), pp. 263-270 (2011). Atrasentan has been shown to be effective in patients with diabetic kidney disease (DKD), significantly reducing the risk of renal events defined as a doubling of serum creatinine or end-stage kidney disease. See, e.g., Heerspink, et al., The Lancet, 393, pp 1937-1947 (2019).

Exemplary endothelin receptor antagonist formulations for use in the treatment of IgAN are described in PCT/US2020/065311, which is hereby incorporated by reference in its entirety.

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor, etc. In some instances, the carrier is an agent that facilitates the delivery of a small molecule drug or antibody to a target cell or tissue. One of skill in the art will recognize that other pharmaceutical carriers are useful in the present disclosure.

The term “expression” as used herein refers the level of protein or mRNA in a mammalian cell.

The term “activity” as used herein refers to one or more activities of a protein, such as binding or enzymatic activity (e.g., one or more of phosphorylation, dephosphorylation, nuclear import, transcriptional activation, transcriptional repression, and/or binding activity to a substrate or a binding partner).

The term “IL-6 signaling” as used herein means the expression and/or activity of one or more proteins in a signaling pathway beginning with activation of an IL-6 receptor and ending in gene expression. Non-limiting examples of proteins in a signaling pathway beginning with activation of an IL-6 receptor and ending in gene expression include an IL-6 receptor, JAK, STAT3, PI3K, Akt/PKB, IKKs, IkBs, NF-kB, MAPK, Ras, Raf, MEK, and ERK.

The term “NF-kB signaling” as used herein means the expression and/or activity of one or more of IKKα, IKKβ, IkB, and NF-kB, and/or one or more genes upregulated by activity of NF-kB (e.g., one or more of TNF-α, IL-1, CAM, COX-2, and iNOS).

The term “PDGF signaling” as used herein means the expression and/or activity of one or more of PDGF receptor, PKC, PI3K, Src, Ras, ERK1/2, Rho, Rac, Akt, mTOR. NAPDH oxidase, MAPK, and cPLA2.

The term “SGLT-2 inhibitor” as used herein refers to a compound that inhibits the sodium-glucose co-transporter-2 (SGLT-2). SGLT-2 inhibitors disrupt reabsorption of glucose by the kidneys and thus exert a glucose-lowering effect. By enhancing glucosuria, independently of insulin, SGLT-2 inhibitors have been shown to treat Type 2 diabetes and to improve cardiovascular outcomes. See Wright, 2001, Am. J. Physiol. Renal Physiol. 280:F10; and Scheen, 2018, Circ. Res. 122:1439. In some embodiments, the term “SGLT-2 inhibitor” refers to compounds whose primary effect is inhibition of SGLT-2, but is not limited to compounds that only inhibit SGLT-2, thus including compounds that have other activities in addition to SGLT-2 inhibition (e.g., SGLT-1 inhibition).

In some embodiments, SGLT-2 inhibitors include compounds of a class of drugs known as gliflozins. In some embodiments, SGLT-2 inhibitors include compounds that are approved as SGLT-2 inhibitors by a regulatory agency such as the FDA or EMA. Non-limiting examples of SGLT-2 inhibitors include bexagliflozin, canagliflozin (INVOKANA®), dapagliflozin (FARXIGA®), empagliflozin (JARDIANCE®), ertugliflozin (STEGLATRO™) ipragliflozin (SUGLAT®), luseogliflozin (LUSEFI®), remogliflozin, sergliflozin, licogliflozin, sotagliflozin (ZYNQUISTA™), and tofogliflozin.

In some embodiments, the SGLT-2 inhibitors include, but are not limited to dapagliflozin, canagliflozin, ipragliflozin, empaglifozin, bexagliflozin, licogliflozin, janagliflozin (XZP-5695), tofogliflozin, ertugliflozin, henagliflozin (SHR-3824), enavogliflozin (DWP-16001), TA-1887 (3-(4-cyclopropylbenzyl)-4-fluoro-1-(Q-D-glucopyranosyl)-1H-indole), indole-N-glycoside 18 (3-(4-ethylbenzyl)-1-(O-D-glucopyranosyl)-1H-indole), sotagliflozin, luseogliflozin, sergliflozin etabonate (ethyl carbonate), remogliflozin, remogliflozin etabonate, and T-1095 (((2R,3S,4S,5R,6S)-6-(2-(3-(benzofuran-5-yl)propanoyl)-3-hydroxy-5-methylphenoxy)-3,4,5-trihydroxytetrahydro-2H-pyran-2-yl) etabonate).

In some embodiments, the SGLT-2 inhibitors include C-glycosides such as dapagliflozin, canagliflozin, ipragliflozin, empaglifozin, bexagliflozin, licogliflozin, janagliflozin (XZP-5695), tofogliflozin, ertugliflozin, henagliflozin (SHR-3824), enavogliflozin (DWP-16001). In some embodiments, the SGLT-2 inhibitors include C-glycosides with a bicyclic or spiro pyran group, such as tofogliflozin, ertugliflozin, and henagliflozin (SHR-3824). In some embodiments, the SGLT-2 inhibitors include C-glycosides that do not have a bicyclic or spiro pyran group, such as dapagliflozin, canagliflozin, ipragliflozin, empaglifozin, bexagliflozin, licogliflozin, janagliflozin (XZP-5695), and enavogliflozin (DWP-16001).

In some embodiments, the SGLT-2 inhibitors include N-glycosides such as TA-1887 (3-(4-cyclopropylbenzyl)-4-fluoro-1-(p-D-glucopyranosyl)-1H-indole) and indole-N-glycoside 18 (3-(4-ethvlbenzyl)-1-(P-D-glucopyranosyl)-1H-indole).

In some embodiments, the SGLT-2 inhibitors include 2-methylthio-C-glycosides, such as sotagliflozin.

In some embodiments, the SGLT-2 inhibitors include thiopyran-C-glycosides, such as luseogliflozin.

In some embodiments, the SGLT-2 inhibitors include 0-glycosides and O-glycoside prodrugs, such as sergliflozin etabonate (ethyl carbonate), remogliflozin, remogliflozin etabonate, and T-1095 (((2R,3S,4S,5R,6S)-6-(2-(3-(benzofuran-5-yl)propanoyl)-3-hydroxy-5-methylphenoxy)-3,4,5-trihydroxytetrahydro-2H-pyran-2-yl) etabonate).

SGLT-2 inhibitors include pharmaceutically acceptable salts, solvates, complexes, and salts of solvates thereof, for example, “dapagliflozin” includes salts of dapagliflozin (such as the hydrochloride salt) as well as solvates (such as the propylene glycol hydrate): likewise. “canagliflozin” includes solvates (such as canagliflozin hemihydrate) and salts of solvates (such as the hydrochloride salt of the hydrate). Similarly, henagliflozin (SHR-3824) and dapagliflozin include complexes (such as the complexes henagliflozin proline and dapagliflozin proline, respectively).

In some embodiments, an SGLT-2 inhibitor, as defined herein, includes any compound exhibiting SGLT-2 inhibition activity. In some embodiments, an SGLT-2 inhibitor is selective for SGLT-2 over SGLT-1, for example, by having about 2-fold, about 5-fold, about 10-fold, about 20-fold, about 50-fold, about 100-fold, about 200-fold, about 300-fold, about 400-fold, about 500-fold, about 750-fold, about 1,000-fold, about 1,250-fold, about 1,500-fold, about 1,750-fold, about 2,000-fold, about 2,500-fold, or any value in between, greater activity against SGLT-2 than against SGLT-1. Exemplary SGLT-2 inhibitors can exhibit inhibition activity (IC₅₀) against SGLT-2 of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, SGLT-2 inhibitors can exhibit inhibition activity (Icso) against SGLT-2 of less than about 25 nM, less than about 10 nM, less than about 5 nM, or less than about 1 nM as measured in an assay as provided herein. An exemplary assay for determining SGLT-2 inhibitory activity is described in Ryan, et al., Kidney International, Vol. 45, pp. 48-57 (1994). Briefly, CHO cells are stably transfected with cDNA encoding human SGLT-2 (GenBank #M95549). Cells are washed and then incubated with 10 M [¹⁴C]alpha-methyl glucopyranoside (AMG), and 10 M inhibitor. The uptake of [¹⁴C]AMG is quenched with cold buffer containing phlorizin, and cells are lysed. Suitable reagents are then used to quantify the uptake of [¹⁴C]AMG.

SGLT-2 inhibitors include pharmaceutically acceptable salts, solvates, complexes, and salts of solvates thereof, for example, “dapagliflozin” includes salts of dapagliflozin (such as the hydrochloride salt) as well as solvates (such as the propylene glycol hydrate): likewise. “canagliflozin” includes solvates (such as canagliflozin hemihydrate) and salts of solvates (such as the hydrochloride salt of the hydrate). Similarly, henagliflozin (SHR-3824) and dapagliflozin include complexes (such as the complexes henagliflozin proline and dapagliflozin pro line, respectively).

Exemplary SGLT-2 inhibitor formulations for use in the treatment of IgAN are described in PCT/US2008/057888, which is hereby incorporated by reference in its entirety.

As used herein, when a subject is described as having “controlled serum glucose levels”, it means the subject has a serum glucose level within the normal or healthy ranges. In some embodiments, the subject has a fasting serum glucose level of between about 70 mg/dL and about 130 mg/dL. For example, the subject has been determined to have a fasting serum glucose level of below about 130 mg/dL, 125 mg/dL, 120 mg/dL, 115 mg/dL, 110 mg/dL. 105 mg/dL, 100 mg/dL, 95 mg/dL, 90 mg/dL. 85 mg/dL, 80 mg/dL, or 75 mg/dL.

As used in the methods described herein, the term “reducing” refers to a reduction in the indicated parameter relative to the baseline measurement (or measurements) of the same parameter in the subject taken prior to the initiation of administration with an endothelin receptor antagonist, or a pharmaceutically acceptable salt thereof, and the APRIL binding antibody or antigen-binding fragment thereof, or a reduction in the indicated parameter relative to the baseline measurement (or measurements) of the same parameter in a healthy subject (for example, a subject that does not have IgA nephropathy). Similarly, the term “increasing,” as used herein, refers to an increase in the indicated parameter relative to the baseline measurement (or measurements) of the same parameter in the subject taken prior to the initiation of administration with an endothelin receptor antagonist, or a pharmaceutically acceptable salt thereof, and the APRIL binding antibody or antigen-binding fragment thereof, or an increase in the indicated parameter relative to the baseline measurement (or measurements) of the same parameter in a healthy subject (for example, a subject that does not have IgA nephropathy).

The term “glomerular filtration rate (GFR)” is defined as the volume of fluid filtered from the renal (kidney) glomerular capillaries into the Bowman's capsule per unit time. It is indicative of overall kidney function. The glomerular filtration rate (GFR) can be calculated by measuring any chemical that has a steady level in the blood, and is freely filtered but neither reabsorbed nor secreted by the kidneys. The rate therefore measured is the quantity of the substance in the urine that originated from a calculable volume of blood. The GFR is typically recorded in units of volume per time, e.g., milliliters per minute and the formula below can be used: GFR=(Urine Concentration ×Urine Volume)/Plasma Concentration. The GFR can be determined by injecting inulin into the plasma. Since inulin is neither reabsorbed nor secreted by the kidney after glomerular filtration, its rate of excretion is directly proportional to the rate of filtration of water and solutes across the glomerular filter. A normal value is: GFR=90-125 mL/min/1.73 m², in particular GFR=100-125 mL/min/1.73 m². Other principles to determine GFR involve measuring 51Cr-EDTA, [125I]iothalamate or iohexol. The “estimated glomerular filtration rate (eGFR)” is defined as derived at screening from serum creatinine values based on e.g., the Chronic Kidney Disease Epidemiology Collaboration (CKD-EPI) equation, the Cockcroft-Gault formula or the Modification of Diet in Renal Disease (MDRD) formula, which are all known in the art. “Stabilizing eGFR” as used herein means reducing the rate of decrease of eGFR and/or attenuating the rate of decline of eGFR. For example, the rate of decline of eGFR can be attenuated by at least about 20%: by at least about 30%: by at least about 40%; by at least about 50%; by at least about 60%; by at least about 70%; by at least about 80%; by at least about 90%; or by at least about 95%; or any value in between after treatment with an endothelin receptor antagonist, or a pharmaceutically acceptable salt thereof, and the APRIL binding antibody or antigen-binding fragment thereof. This attenuation can be after treatment, for example, for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between. In some embodiments, the subject has been treated with an endothelin receptor antagonist, or a pharmaceutically acceptable salt thereof, and the APRIL binding antibody or antigen-binding fragment thereof for between about 15 days and about 30 days. In some embodiments, the subject has been treated with an endothelin receptor antagonist, or a pharmaceutically acceptable salt thereof, and the APRIL binding antibody or antigen-binding fragment thereof for between about 6 months and about 1 year.

“ESRD” is the abbreviation for end-stage renal disease. As used herein, the onset of ESRD is defined as the time point when the subject has an eGFR of below about 15 mL/min/1.73 m² and/or when the subject has initiated chronic dialysis. When a subject is defined to be “at a high risk of progression to ESRD”, the subject has >1 g/day protein in the urine and/or eGFR <60 for at least about 3 months before the first administration of an endothelin receptor antagonist, or a pharmaceutically acceptable salt thereof, and the APRIL binding antibody or antigen-binding fragment thereof.

“IgA-nephropathy associated disease flares” as used herein refer to disease flares associated with hematuria, worsening proteinuria, systemic manifestations, and declines in eGFR. Other symptoms associated with disease flares include increased edema, fatigue, increased hematuria, gross hematuria, and other symptoms which generally negatively impact disease progression.

As used herein, when a subject is described to “maintain a potassium level within the normal physiologic range”, the subject has a blood potassium level of from about 3.5 mEq/L to about 5.2 mEq/L.

As used herein, when a subject is described to “maintain a sodium level within the normal physiologic range”, the subject has a blood sodium level of from about 135 to about 145 mEq/L.

As used herein, the term “proteinuria” refers to the presence of protein in the urine in excess of normal levels. “Proteinuria” includes “albuminuria” and “microalbuminuria”. Normal human levels of protein appear in the urine in the range of about 0 to 30 mg/L, although for any given urine sample, the level may reach about 80 mg/L. For a 24 hour urine collection, normal human levels of urinary protein are in the range of about 0 to 150 mg. Proteinuria can be indicated by the ratio of total protein/creatinine in the urine (UPCR), or by the ration of a specific protein, such as a urinary albumin/creatinine ratio (ACR) of greater than about 30 mg/g. Typically, the urinary UACR value in mg/g approximately equals to the albumin excretion by the subject in mg/day. Proteinuria, including albuminuria and microalbuminuria, often leads to or is indicative of a disease, but is not limited to production of a disease. Proteinuria is intended to encompass all forms of proteinuria, including but not limited to physiological proteinuria; functional proteinuria; and athletic proteinuria, which relates to a form of functional proteinuria following excessive muscular exertion. Further, proteinuria covers benign proteinuria (also known as “essential” proteinuria), which refers to types or proteinuria that are not the result of pathologic changes in the kidneys. Proteinuria also covers pathologic proteinuria, for example levels of protein in the urine greater than normal physiological levels.

As used herein, the term “albuminuria” (also known as “macroalbuminuria”) refers to the presence of albumin in the urine in excess of normal levels. Since urinary protein is predominantly albumin, normal human levels of urinary UACR are in the range of about 0 to 30 mg/mmol. As used herein, the term “microalbuminuria” refers to the presence of albumin in the urine, excreted at a rate of about 20 to 200 pg/min or at a level of about 30 to 300 mg/L in humans. When defined by the urinary ACR, “microalbuminuria” refers to a urinary UACR of greater than about 30 mg/g, or a urinary UACR of about 3.5 mg/mmol or greater for women and about 2.5 mg/mmol or greater for men. Microalbuminuria is often an early warning of kidney disease, but can also be present for other reasons.

As used herein, the term “hematuria” refers to the presence of blood in the urine. It may present as macroscopic hematuria (visible traces of blood cells) or microscopic hematuria (microscopic traces of blood) within the urine. A confirmed indication of microhematuria is defined as 3 or more red blood cells present per microscopic high-powered field (HPF) on a minimum of 3 properly collected urine samples. Microhematuria may also be detected by urine dipstick (colorimetric comparison estimate) at clinic. Hematuria (either microscopic or macroscopic) may be asymptomatic (no additional symptoms associated with hematuria) or symptomatic. Additional symptoms include dysuria (painful urination), a feeling of incomplete emptying of the bladder or increased frequency or urination, or flank pain.

“ALT” as used herein refers to alanine transaminase. “AST” as used herein refers to aspartate transaminase.

Unless otherwise stated, any reference to an amount of an endothelin receptor antagonist in this disclosure is based on the free equivalent weight of the endothelin receptor antagonist. For example, 0.75 mg of atrasentan refers to 0.75 mg of atrasentan in the free form or an equivalent amount of a salt form of atrasentan.

The disclosure also includes combination therapy using an anti-APRIL antibody or antigen binding fragment thereof. The antibodies described herein are exemplified using the anti-hAPRIL antibody having amino acid sequence of SEQ ID NO: 28 for the heavy chain and SEQ ID NO: 30 for the light chain (also referred to as VH14_1G.VL15, or as used in clinical trials is also referred to as BION-1301). In some instances, the anti-APRIL antibody or antigen binding fragment thereof is VIS649. In some instances, the anti-APRIL antibody or antigen binding fragment thereof comprises a heavy chain complementarity-determining region-1 (HC CDR1) comprising an amino acid sequence having at least 90% sequence identity to SEQ ID NO:1; a heavy chain complementarity-determining region-2 (HC CDR2) comprising an amino acid sequence having at least 90% sequence identity to SEQ ID NO:2; a heavy chain complementarity-determining region-3 (HC CDR3) comprising an amino acid sequence having at least 90% sequence identity to SEQ ID NO:3; a light chain complementarity-determining region-1 (LC CDR1) comprising an amino acid sequence having at least 90% sequence identity to SEQ ID NO:4; a light chain complementarity-determining region-2 (LC CDR2) comprising an amino acid sequence having at least 90% sequence identity to SEQ ID NO:5; and a light chain complementarity-determining region-3 (LC CDR3) comprising an amino acid sequence having at least 90% sequence identity to SEQ ID NO:6. In some instances, the heavy and light chain CDR sequences are 95% identical to SEQ ID NOs: 1-6. In some instances, the heavy and light chain CDR sequences comprise SEQ ID NOs: 1-6. In some instances, the heavy and light chain CDR sequences consist of SEQ ID NOs: 1-6. In some instances, the APRIL binding antibody or antigen-binding fragment thereof comprises a heavy chain variable region comprising an amino acid sequence having at least 90% sequence identity to SEQ ID NOs: 8, 10, 12, 14, 16, 18, 20, 22, or 24. In some instances, the APRIL binding antibody or antigen-binding fragment thereof comprises a heavy chain variable region comprising an amino acid sequence of SEQ ID NOs: 8, 10, 12, 14, 16, 18, 20, 22, or 24. In some instances, the APRIL binding antibody or antigen-binding fragment thereof comprises a light chain variable region comprising an amino acid sequence having at least 90%, 95%, or 99% sequence identity to SEQ ID NO: 26. In some instances, the APRIL binding antibody or antigen-binding fragment thereof comprises a light chain variable region comprising an amino acid sequence of SEQ ID NO: 26. In some instances, the APRIL binding antibody or antigen-binding fragment thereof comprises a heavy chain variable region comprising an amino acid sequence of SEQ ID NOs: 8, 10, 12, 14, 16, 18, 20, 22, or 24 and a light chain variable region comprising an amino acid sequence of SEQ ID NO: 26. In some instances, the APRIL binding antibody or antigen-binding fragment thereof comprises a heavy chain variable region comprising an amino acid sequence of SEQ ID NO: 24 and a light chain variable region comprising an amino acid sequence of SEQ ID NO: 26. In some instances, the APRIL binding antibody or antigen-binding fragment thereof comprises a heavy chain comprising an amino acid sequence having at least 90% sequence identity to SEQ ID NO: 28. In some instances, the APRIL binding antibody or antigen-binding fragment thereof comprises a heavy chain comprising an amino acid sequence of SEQ ID NO: 28. In some instances, the APRIL binding antibody or antigen-binding fragment thereof comprises alight chain comprising an amino acid sequence having at least 90% sequence identity to SEQ ID NO: 30. In some instances, the APRIL binding antibody or antigen-binding fragment thereof comprises a light chain comprising an amino acid sequence of SEQ ID NO: 30. In some instances, the APRIL binding antibody or antigen-binding fragment thereof comprises a heavy chain comprising an amino acid sequence of SEQ ID NO: 28 and a light chain comprising an amino acid sequence of SEQ ID NO: 30. In some instances, the APRIL binding antibody or antigen-binding fragment thereof comprises a heavy chain secretion leader comprising an amino acid sequence having at least 90% sequence identity to SEQ ID NO: 32. In some instances, the APRIL binding antibody or antigen-binding fragment thereof comprises a heavy chain secretion leader comprising an amino acid sequence of SEQ ID NO: 32. In some instances, the APRIL binding antibody or antigen-binding fragment thereof comprises a light chain secretion leader comprising an amino acid sequence having at least 90% sequence identity to SEQ ID NO: 34. In some instances, the APRIL binding antibody or antigen-binding fragment thereof comprises a light chain secretion leader comprising an amino acid sequence of SEQ ID NO: 34. In some instances, the APRIL binding antibody or antigen-binding fragment thereof is generated from a nucleic acid comprising one of SEQ ID NOs: 7, 9, 11, 13, 15, 17, 19, 21, or 23. In some instances, the APRIL binding antibody or antigen-binding fragment thereof is generated from a nucleic acid comprising SEQ ID NO: 25, 27, 29, 31, or 33.

This antibody blocks the binding of human APRIL to human B cell maturation antigen (BCMA) and transmembrane activator and calcium modulator and cyclophilin ligand interactor (TACI), and has been shown to significantly reduce the levels of IgA in healthy volunteers. This reduction in IgA levels is expected to be similar in subjects having IgA nephropathy, and is therefore expected to have a significant therapeutic benefit. Additional features and discussion of antibodies useful in the formulation and methods described herein can be found in PCT Publ. No. WO2016/110587.

The anti-APRIL antibody or antigen binding fragment thereof can be called “BION-1301,” which refers to the IgG4 humanized monoclonal antibody having CAS Registry No. 2642175-46-8. The disease-modifying potential of BION-1301 is mediated by blocking APRIL binding to its two main receptors, transmembrane activator and calcium modulator and cyclophilin ligand interactor (TACI) and B cell maturation antigen (BCMA). Elevated APRIL levels are associated with the pathogenesis of IgA nephropathy. Hence, mAb-mediated neutralization of APRIL to reduce receptor activation is a promising mechanism for the treatment of IgA nephropathy. BION-1301 does not bind BAFF (required for normal B cell maintenance), which is in contrast to the approved APRIL antagonist atacicept which binds APRIL and BAFF.

BION-1301 has been studied in adults with relapsed or refractory multiple myeloma as well as healthy volunteers (HVs) and adults with IgAN. Since the initiation of the BION-1301 clinical program through 26 Jul. 2022, a total of 153 adults have been enrolled in 5 clinical studies. Interim results from an ongoing Phase 1/2 clinical study evaluating IV administration of BION 1301 450 mg Q2W in patients with IgAN demonstrated that BION-1301 treatment was well-tolerated and resulted in sustained reductions in free (unbound) APRIL. Gd-IgA1, and proteinuria (as measured by areduction in UPCR). Following IV infusion, serum BION-1301 concentrations appeared comparable to those observed in healthy volunteers at the same dose and schedule. Following at least 24-weeks of IV administration, all subjects who transitioned to SC dosing at 600 mg Q2W maintained similar BION-1301 exposures, biomarker responses and proteinuria reductions. On average, patients treated with BION-1301 demonstrated a reduction in 24-hour UPCR which was evident by 3 months and continue to decline beyond 1 year, thereby providing preliminary clinical evidence of efficacy. The magnitude of the proteinuria reductions observed are predicted to translate into clinically meaningful preservation of eGFR and significantly improved long-term kidney outcomes.

Exemplary BION-1301 formulations for use in the treatment of IgAN are described in PCT/US2021/035011, which is hereby incorporated by reference in its entirety.

Within the description of the present disclosure at least 90% sequence similarity should be understood as meaning in some instances at least 95%, such as at least 99% sequence similarity.

As used herein, “sequence similarity” refers to the extent to which individual nucleotide or peptide sequences are alike. The extent of similarity between two sequences is based on the extent of identity combined with the extent of conservative changes. The percentage of “sequence similarity” is the percentage of amino acids or nucleotides which is either identical or conservatively changed viz. “sequence similarity”=(% sequence identity)+(% conservative changes).

For the purpose of this disclosure, “conservative changes” and “identity” are considered to be species of the broader term “similarity”. Thus, whenever the term sequence “similarity” is used it embraces sequence “identity” and “conservative changes”. According to certain embodiments the conservative changes are disregarded and the % sequence similarity refers to % sequence identity.

The term “sequence identity” is known to the skilled person. In order to determine the degree of sequence identity shared by two amino acid sequences or by two nucleic acid sequences, the sequences are aligned for optimal comparison purposes (e.g., gaps can be introduced in the sequence of a first amino acid or nucleic acid sequence for optimal alignment with a second amino or nucleic acid sequence). Such alignment may be carried out over the full lengths of the sequences being compared. Alternatively, the alignment may be carried out over a shorter comparison length, for example over about 20, about 50, about 100 or more nucleic acids/bases or amino acids.

The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The degree of identity shared between sequences is typically expressed in terms of percentage identity between the two sequences and is a function of the number of identical positions shared by identical residues in the sequences (i.e., % identitv=number of identical residues at corresponding positions/total number of positions×100). In some instances, the two sequences being compared are of the same or substantially the same length.

The percentage of “conservative changes” may be determined similar to the percentage of sequence identity. However, in this case changes at a specific location of an amino acid or nucleotide sequence that are likely to preserve the functional properties of the original residue are scored as if no change occurred.

For amino acid sequences the relevant functional properties are the physico-chemical properties of the amino acids. A conservative substitution for an amino acid in a polypeptide of the disclosure may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without substantially altering the activity of a protein, particularly in regions of the protein that are not directly associated with biological activity (see, e.g., Watson, et al., Molecular Biology of the Gene, The Benjamin/Cumings Pub. Co., p. 224 (4^{th} Edition 1987)). For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr and vice versa so that a free —OH is maintained; and Gln for Asn and vice versa to maintain a free —NH₂.

For nucleotide sequences the relevant functional properties is mainly the biological information that a certain nucleotide carries within the open reading frame of the sequence in relation to the transcription and/or translation machinery. It is common knowledge that the genetic code has degeneracy (or redundancy) and that multiple codons may carry the same information in respect of the amino acid for which they code. For example, in certain species the amino acid leucine is coded by UUA. UUG, CUU. CUC, CUA, CUG codons (or TTA, TTG, CTT, CTC, CTA, CTG for DNA), and the amino acid serine is specified by UCA, UCG, UCC, UCU, AGU, AGC (or TCA, TCG, TCC, TCT, AGT, AGC for DNA). Nucleotide changes that do not alter the translated information are considered conservative changes.

For the present disclosure, BLAST (Basic Local Alignment Tool) can be used to determine the percentage identity and/or similarity between nucleotide or amino acid sequences. Queries using the BLASTn, BLASTp, BLASTx, tBLASTn and tBLASTx programs of Altschul et al. (1990) may be posted via the online versions of BLAST accessible via http://www.ncbi.nlm.nih.gov. Alternatively a standalone version of BLAST (e.g., version 2.2.29 (released 3 Jan. 2014)) downloadable also via the NCBI internet site may be used. BLAST queries are performed with the following parameters. To determine the percentage identity and/or similarity between amino acid sequences: algorithm: blastp; word size: 3; scoring matrix: BLOSUM62; gap costs: Existence: 11, Extension: 1; compositional adjustments: conditional compositional score matrix adjustment; filter: offt mask: off. To determine the percentage identity and/or similarity between nucleotide sequences: algorithm: blastn; word size: 11; max matches in query range: 0; match/mismatch scores: 2, -3; gap costs: Existence: 5, Extension: 2; filter: low complexity regions; mask: mask for lookup table only.

The percentage of “conservative changes” may be determined similar to the percentage of sequence identity with the aid of the indicated algorithms and computer programs. Some computer programs, e.g., BLASTp, present the number/percentage of positives (=similarity) and the number/percentage of identity. The percentage of conservative changes may be derived therefrom by subtracting the percentage of identity from the percentage of positives/similarity (percentage conservative changes=percentage similarity-percentage identity).

According to a further aspect, the disclosure relates to methods using an isolated polynucleotide encoding a VH domain and/or a VL domain of an antibody, or a heavy chain and/or light chain of the antibody, according to the disclosure. A polynucleotide sequence encoding the VH domain in some instances is a polynucleotide sequence having at least 90% sequence similarity with a polynucleotide sequence selected from the group consisting of SEQ ID NO: 7, 9, 11, 13, 15, 17, 19, 21 and 23, in some instances SEQ ID NO: 13, 15 or 23, and in some instances SEQ ID NO: 23. A polynucleotide sequence encoding the VL domain in some instances is a polynucleotide sequence having at least 90% sequence similarity with a polynucleotide sequence of SEQ ID NO: 25. A polynucleotide sequence encoding the heavy chain in some instances is a polynucleotide sequence having at least 90% sequence similarity with a polynucleotide sequence of SEQ ID NO: 27. A polynucleotide sequence encoding the light chain in some instances is a polynucleotide sequence having at least 90% sequence similarity with a polynucleotide sequence of SEQ ID NO: 29.

The disclosure further relates to an expression unit comprising a number of expression vectors, comprising a number of polynucleotides according to the disclosure under the control of suitable regulatory sequences, wherein the number of polynucleotides encode the VH domain or heavy chain and the VL domain or light chain of an antibody according to the disclosure. The expression unit may be designed such that the polynucleotide sequence coding for the VH domain or heavy chaining and the polynucleotide sequence coding for VL domain or light chain may be on the same expression vector. Thus, the expression unit may comprise a single vector. Alternatively, the polynucleotide sequence coding for the VH domain or heavy chain and the polynucleotide sequence coding for the VL domain or light chain may be on different expression vectors.

A further aspect of the disclosure relates to a host cell comprising a number of polynucleotides of the disclosure and/or an expression unit of the disclosure. The expression unit in some instances is an expression unit comprising an expression vector comprising both a polynucleotide sequence coding for the VH domain or heavy chain and a polynucleotide sequence coding for the VL domain or light chain.

A humanized APRIL antagonistic monoclonal antibody (anti-APRIL antibody, as described herein) is in development for the treatment of IgAN, having undergone clinical trials in healthy volunteers (see clinicaltrials.gov NCT03945318). Blockade of APRIL by anti-hAPRIL antibody has been shown to significantly lower IgA and IgM and to a lesser extent IgG in healthy cynomolgus monkeys, and has shown similar results in the healthy human volunteers. In addition, this blockade reduced Gd-IgAQ1 in healthy human volunteers. Consequently, it is expected that blockade of APRIL in patients with IgAN will lead to a reduction in levels of IgA, IgG, and IgM and corresponding reductions in gd-IgA1, auto-antibodies to gd-IgA1, immune complex deposition and renal damage.

Myette et al. (2019, Kidney Intemational 96(1):104-116) demonstrates the efficacy of a mouse anti-APRIL antibody in a mouse model for IgA nephropathy, and the human antibody VIS649 is part of a Phase 2 clinical trial (clinicaltrials.gov NCT04287985).

The term “antibody” refers to any form of antibody that exhibits the desired biological activity, such as inhibiting binding of a ligand to its receptor, or by inhibiting ligand-induced signaling of a receptor. In the present case the biological activity comprises blocking of the binding of APRIL to its receptors BCMA and/or TAC. Thus, “antibody” is used in the broadest sense and specifically covers, but is not limited to, monoclonal antibodies (including full length monoclonal antibodies) and multispecific antibodies (e.g., bispecific antibodies) such as based on the DuobodyC technology (Genmab) or Hexabody® technology (Genmab) or antibody fragment.

“Antibody fragment” and “antibody binding fragment” mean antigen-binding fragments and analogues of an antibody, typically including at least a portion of the antigen binding or variable regions (e.g., one or more CDRs) of the parental antibody. An antibody fragment retains at least some of the binding specificity of the parental antibody. Typically, an antibody fragment retains at least 10% of the parental binding activity when that activity is expressed on a molar basis. In some instances, an antibody fragment retains at least 20%, 50%, 70%, 80%, 90%, 95% or 100% or more of the parental antibody's binding affinity for the target. Examples of antibody fragments include, but are not limited to, Fab, Fab′, F(ab′)2, and Fv fragments; diabodies; linear antibodies; single-chain antibody molecules, e.g., sc-Fv, unibodies (technology from Genmab); nanobodies (technology from Ablynx); domain antibodies (technology from Domantis); and multispecific antibodies formed from antibody fragments. Engineered antibody variants are reviewed in Holliger and Hudson, 2005, Nat. Biotechnol. 23:1126-1136.

An “Fab fragment” is comprised of one light chain and the CH1 and variable regions of one heavy chain. The heavy chain of a Fab molecule cannot form a disulfide bond with another heavy chain molecule.

An “Fc” region contains two heavy chain fragments comprising the CH1 and CH2 domains of an antibody. The two heavy chain fragments are held together by two or more disulfide bonds and by hydrophobic interactions of the CH3 domains.

An “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form a F(ab′)2 molecule.

An “F(ab′)2 fragment” contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains.

The “Fv region” comprises the variable regions from both the heavy and light chains, but lacks the constant regions.

A “single-chain Fv antibody” (or “scFv antibody”) refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. For a review of scFv, see Pluckthun, 1994, The Pharmacology of Monoclonal Antibodies, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “diabody” is a small antibody fragment with two antigen-binding sites. The fragment comprises a heavy chain variable domain (VH) connected to a light chain variable domain (VL) in the same polypeptide chain (VH-VL or VL-VH). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al., 1993, Proc. Natl. Acad. Sci. USA 90: 6444-6448.

“Duobodies” are bispecific antibodies with normal IgG structures (Labrijn et al., 2013, Proc. Natl. Acad. Sci. USA 110 (13): 5145-5150).

“Hexabodies” are antibodies that while retaining regular structure and specificity have an increased killing ability (Diebolder et al., 2014, Science 343(6176):1260-3).

A “domain antibody fragment” is an immunologically functional immunoglobulin fragment containing only the variable region of a heavy chain or the variable region of a light chain. In some instances, two or more VH regions are covalently joined with a peptide linker to create a bivalent domain antibody fragment. The two VH regions of a bivalent domain antibody fragment may target the same or different antigens.

An antibody fragment of the disclosure may comprise a sufficient portion of the constant region to permit dimerization (or multimerization) of heavy chains that have reduced disulfide linkage capability, for example where at least one of the hinge cysteines normally involved in inter-heavy chain disulfide linkage is altered as described herein. In another embodiment, an antibody fragment, for example one that comprises the Fc region, retains at least one of the biological functions normally associated with the Fc region when present in an intact antibody, such as FcRn binding, antibody half-life modulation, ADCC (antibody dependent cellular cytotoxicity) function, and/or complement binding (for example, where the antibody has a glycosylation profile necessary for ADCC function or complement binding).

The term “chimeric” antibody refers to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. See, e.g., U.S. Pat. No. 4,816,567 and Morrison et al., Proc. Nat. Acad. Sci. USA, Vol. 81, pp. 6851-6855 (1984).

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The humanized forms of rodent antibodies will essentially comprise the same CDR sequences of the parental rodent antibodies, although certain amino acid substitutions may be included to increase affinity, increase stability of the humanized antibody, or for other reasons.

The antibodies of the present disclosure also include antibodies with modified (or blocked) Fc regions to provide altered effector functions. See, e.g. U.S. Pat. No. 5,624,821; PCT Publ. Nos. WO2003/086310, WO2005/120571, and WO2006/0057702; and Presta, Adv. Drug Delivery Rev. Vol. 58, pp. 640-656 (2006). Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta, J. Allergy Clin. Immunol. Vol. 116, No. 731, pp. 734-35 (2005).

The antibodies of the present disclosure also include antibodies with intact Fc regions that provide full effector functions, e.g., antibodies of isotype IgG1, which induce complement-dependent cytotoxicity (CDC) or antibody dependent cellular cytotoxicity (ADCC) in a targeted cell.

The antibodies may also be conjugated (e.g., covalently linked) to molecules that improve stability of the antibody during storage or increase the half-life of the antibody in vivo. Examples of molecules that increase the half-life are albumin (e.g., human serum albumin) and polyethylene glycol (PEG). Albumin-linked and PEGylated derivatives of antibodies can be prepared using techniques well known in the art. See, e.g. Chapman, 2002, Adv. Drug Deliv. Rev. 54:531-545; Anderson and Tomasi, 1988, J. Immunol. Methods 109:37-42: Suzuki et al., 1984, Biochim. Biophys. Acta 788:248-255; and Brekke and Sandlie, 2003, Nature Rev. 2:52-62.

The term “hypervariable region,” as used herein, refers to the amino acid residues of an antibody which are responsible for antigen-binding. The hypervariable region comprises amino acid residues from a “complementarity determining region” or “CDR,” defined by sequence alignment, for example residues 24-34 (L1), 50-56 (L2) and 89-97 (L3) in the light chain variable domain and 31-35 (H1), 50-65 (H2) and 95-102 (H3) in the heavy chain variable domain (see Kabat et al., 1991, Sequences of proteins of Immunological Interest, 5^{th} Ed. Public Health Service, National Institutes of Health, Bethesda, Md.) and/or those residues from a “hvpervariable loop” (HVL), as defined structurally, for example, residues 26-32 (L1), 50-52 (L2) and 91-96 (L3) in the light chain variable domain and 26-32 (H1), 53-55 (H2) and 96-101 (H3) in the heavy chain variable domain (see Chothia and Leskl, 1987, J. Mol. Biol. 196:901-917).

“Framework” or “FR” residues or sequences are those variable domain residues or sequences other than the CDR residues as herein defined.

The antibody of the disclosure according to certain embodiments may be an isolated antibody. An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In some embodiments, the antibody will be purified (1) to greater than 95% by weight of antibody as determined by the Lowry method, and in some instances more than 99% by weight. (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, in some instances, silver stain. Isolated antibody includes the antibody in situ within recombinant cells since at least one component of the antibody's natural environment will not be present. Ordinarily, however, isolated antibody will be prepared by at least one purification step.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The term “monoclonal antibody” when used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al., 1975, Nature 256:495, or may be made by recombinant DNA methods (see, for example, U.S. Pat. No. 4,816,567). The “monoclonal antibodies” may also be isolated from phage antibody libraries using the techniques described in Clackson et al., 1991, Nature 352:624-628 and Marks et al., 1991, J. Mol. Biol. 222:581-597, for example. The monoclonal antibodies herein specifically include “chimeric” antibodies.

As used herein, the term “immune cell” includes cells that are of hematopoietic origin and that play a role in the immune response. Immune cells include lymphocytes, such as B cells and T cells, natural killer cells, myeloid cells, such as monocytes, macrophages, eosinophils, mast cells, basophils, and granulocytes.

As used herein, a sequence “variant” or “variant sequence” refers to a sequence that differs from the disclosed sequence at one or more amino acid residues but which retains the biological activity of the parent molecule. The disclosure includes the variants of antibodies explicitly disclosed by the various sequences. For the VH domain CDR1. CDR2 and CDR3 sequences, according to some embodiments, variant sequences may comprise up to 6 amino acid substitutions, such as 1, 2, 3, 4, 5 or 6 amino acid substitutions, for the CDR1, CDR2 and CDR3 sequences taken together. Similarly for the VL domain CDR1, CDR2 and CDR3 sequences, according to some embodiments, variant sequences may comprise up to 6 amino acid substitutions, such as 1, 2, 3, 4, 5 or 6 amino acid substitutions, for the CDR1. CDR2 and CDR3 sequences taken together.

“Conservatively modified variants” or “conservative amino acid substitution” refers to substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al., Molecular Biology of the Gene, The Benjamin/Cumings Pub. Co., p. 224 (4^{th} Edition 1987)).

“Specifically binds”, when referring to a ligand/receptor, antibody/antigen, or other binding pair, indicates a binding reaction which is determinative of the presence of the protein, e.g., APRIL, in a heterogeneous population of proteins and/or other biologics. Thus, under designated conditions, a specified ligand/antigen binds to a particular receptor/antibody and does not bind in a significant amount to other proteins present in the sample.

The antibody DNA also may be modified, for example, by substituting the coding sequence for human heavy- and light-chain constant domains in place of the homologous murine sequences (U.S. Pat. No. 4,816,567; Morrison, et al., 1984, Proc. Natl Acad. Sci. USA. 81:6851), or by covalently joining to the immunoglobulin coding sequence all or part of the coding sequence for non-immunoglobulin material (e.g., protein domains). Typically such non-immunoglobulin material is substituted for the constant domains of an antibody, or is substituted for the variable domains of one antigen-combining site of an antibody to create a chimeric bivalent antibody comprising one antigen-combining site having specificity for an antigen and another antigen-combining site having specificity for a different antigen.

Amino acid sequence variants of the anti-human APRIL antibodies of the disclosure are prepared by introducing appropriate nucleotide changes into the coding DNAs, or by peptide synthesis. Such variants include, for example, deletions from, and/or insertions into, and/or substitutions of, residues within the amino acid sequences shown for the anti-APRIL antibodies. Any combination of deletion, insertion, and substitution is made to arrive at the final construct, provided that the final construct possesses the desired characteristics. The amino acid changes also may alter post-translational processes of the anti-APRIL antibodies, such as changing the number or position of glycosylation sites.

Ordinarily, amino acid sequence variants of the anti-APRIL antibodies will have an amino acid sequence having at least 75% amino acid sequence similarity with the original antibody amino acid sequences of either the heavy or the light chain more in some instances at least 80%, more in some instances at least 85%, in some instances at least 90%, and in some instances at least 95%, 98% or 99%. Similarity or homology with respect to this sequence is as defined above.

Antibodies having the characteristics identified herein as being desirable can be screened for increased biologic activity in vitro or suitable binding affinity. To screen for antibodies that bind to the same epitope on human APRIL as hAPRIL.01A, a routine cross-blocking assay such as that described in Antibodies, A Laboratory Manual, Cold Spring Harbor Laboratory, Ed Harlow and David Lane (1988), can be performed. Antibodies that bind to the same epitope are likely to cross-block in such assays, but not all cross-blocking antibodies will necessarily bind at precisely the same epitope since cross-blocking may result from steric hindrance of antibody binding by antibodies bind at overlapping epitopes, or even nearby non-overlapping epitopes.

Alternatively, epitope mapping, e.g., as described in Champe et al., 1995, J. Biol. Chem. 270:1388-1394, can be performed to determine whether the antibody binds an epitope of interest. “Alanine scanning mutagenesis,” as described by Cunningham and Wells, 1989, Science 244: 1081-1085, or some other form of point mutagenesis of amino acid residues in human APRIL may also be used to determine the functional epitope for anti-APRIL antibodies of the present disclosure. Another method to map the epitope of an antibody is to study binding of the antibody to synthetic linear and CLIPS peptides that can be screened using credit-card format mini PEPSCAN cards as described by Slootstra et al. (Slootstra et al., 1996, Mol. Diversity 1: 87-96) and Timmerman et al. (Timmerman et al., 2007, J. Mol. Recognit. 20: 283-299). The binding of antibodies to each peptide is determined in a PEPSCAN-based enzyme-linked immuno assay (ELISA).

Additional antibodies binding to the same epitope as hAPRIL.01A may be obtained, for example, by screening of antibodies raised against APRIL for binding to the epitope, or by immunization of an animal with a peptide comprising a fragment of human APRIL comprising the epitope sequences. Antibodies that bind to the same functional epitope might be expected to exhibit similar biological activities, such as similar APRIL binding and BCMA and TACI blocking activity, and such activities can be confirmed by functional assays of the antibodies.

The antibody can be selected from any class of immunoglobulins, including IgM, IgG, IgD, IgA, and IgE. In some instances, the antibody is an IgG antibody. Any isotvpe of IgG can be used, including IgG1, IgG2, IgG3, and IgG4. Variants of the IgG isotypes are also contemplated. The antibody may comprise sequences from more than one class or isotype. Optimization of the necessary constant domain sequences to generate the desired biologic activity is readily achieved by screening the antibodies using biological assays known in the art or as described herein.

Likewise, either class of light chain can be used in the compositions and methods herein. Specifically, kappa, lambda, or variants thereof are useful in the present compositions and methods.

The antibodies and antibody fragments of the disclosure may also be conjugated with cytotoxic payloads such as cytotoxic agents or radionucleotides such as 99Tc, 90Y. 111In, 32P, 14C. 125I, 3H, 131I, 11C, 150, 13N, 18F, 355, 51Cr, 57To, 226Ra, 60Co, 59Fe, 57Se, 152Eu, 67Cu, 217Ci, 211At, 212Pb, 47Sc, 109Pd, 234Th, and 40K, 157Gd, 55Mn, 52Tr and 56Fe. Such antibody conjugates may be used in immunotherapy to selectively target and kill cells expressing a target (the antigen for that antibody) on their surface. Exemplary cytotoxic agents include ricin, vinca alkaloid, methotrexate, Pseudomonas exotoxin, saporin, diphtheria toxin, cisplatin, doxorubicin, abrin toxin, gelonin and pokeweed antiviral protein.

The antibodies and antibody fragments of the disclosure may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, 152Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

Any method known in the art for conjugating the antibody molecules or protein molecules of the disclosure to the various moieties may be employed, including those methods described by Hunter et al., 1962, Nature 144:945; David et al., 1974, Biochemistry 13:1014; Pain et al., 1981, J. Immunol. Meth. 40:219; and Nygren. J., 1982, Histochem. And Cytochem. 30:407. Methods for conjugating antibodies and proteins are conventional and well known in the art.

Various aspects of the disclosure are described in further detail in the following subsections.

### B. METHODS OF TREATMENT

In a normal and healthy human kidney, expressions of ET-1 and ET-RA are more intense in vascular tissue and less intense in glomerular structures. In contrast, subjects with IgAN show increased expressions of ET-1 and ET-RA in the kidney. In that population, ET-1 expression positively correlates with proteinuria, which is at least partially ameliorated by administration of ACE inhibitors. Indeed, the currently therapy for IgAN is optimization of antihypertensive and antiproteinuric agents (e.g., angiotensin converting enzyme inhibitors and/or angiotensin II receptor blockers), along with a course of corticosteroids, to inhibit disease progression. See, e.g., Penfold et al., Int. J. Nephrol. And Renovascular Dis. 11, pp. 137-148 (2017). However, these combinations of agents may exhibit significant dose-limiting side effects such as hyperkalemia, and further immunosuppression may be necessary in more serious cases.

Clinically, IgAN is diagnosed by kidney biopsy indicating the presence of mesangial cell proliferation and/or matrix expansion (or focal segmental glomerular sclerosis in advanced stages) with predominant mesangial granular deposits of IgA (2+ or more) on immunofluorescence. This pathology is distinct from other progressive kidney diseases such as diabetic nephropathy, which typically present with a diffuse capillary basement membrane thickening with peripheral hyaline PAS-positive nodules, with segmental or global glomerular sclerosis at advanced stages, and thickened arterioles with hyaline deposits. See, e.g., Zanatta, et al., Renal Failure, 34(3), pp. 308-315 (2012).

Accordingly, some embodiments provide a method of treating IgA nephropathy, comprising administering an endothelin receptor antagonist, or a pharmaceutically acceptable salt thereof, and an APRIL binding antibody or an antigen-binding fragment thereof. In some embodiments, administration of an endothelin receptor antagonist, or a pharmaceutically acceptable salt thereof, and an APRIL binding antibody or an antigen-binding fragment thereof, provides synergistic effects such as those described herein.

Some embodiments provide a combination therapy an endothelin receptor antagonist, or a pharmaceutically acceptable salt thereof, and an APRIL binding antibody or an antigen-binding fragment thereof, for treating IgA nephropathy in a subject in need thereof.

In some instances, the endothelin receptor antagonist, or a pharmaceutically acceptable salt thereof, is selected from the group consisting of tezosentan, spysentan, bosentan, sparsentan, macitentan, ambrisentan, sitaxentan, atriopeptine, atrasentan, and pharmaceutically acceptable salts of any of the foregoing, and combinations thereof. In some instances, the endothelin receiptor is atrasentan, or a pharmaceutically acceptable salt thereof. In some instances, the endothelin receptor is sparsentan, or a pharmaceutically acceptable salt thereof.

Some embodiments provide a method of treating IgA nephropathy in a subject in need thereof, comprising administering to the subject atrasentan, or a pharmaceutically acceptable salt thereof, and the APRIL binding antibody or antigen-binding fragment thereof. Some embodiments provide a method of treating IgA nephropathy in a subject in need thereof, comprising administering to the subject sparsentan, or a pharmaceutically acceptable salt thereof, and the APRIL binding antibody or antigen-binding fragment thereof.

Some embodiments provide a method of treating IgA nephropathy in a subject in need thereof, comprising administering to the subject atrasentan, or a pharmaceutically acceptable salt thereof, and BION-1301. Some embodiments provide a method of treating IgA nephropathy in a subject in need thereof, comprising administering to the subject sparsentan, or a pharmaceutically acceptable salt thereof, and BION-1301.

In some instances, the sequences presented in the sequence listing relate to the amino acid sequences and encoding DNA sequences of VH and VL domains and of heavy and light chains of anti-APRIL antibodies for the formulations and methods described herein, including the amino acid sequences and encoding DNA sequences of the heavy and light chains of the antibody described herein. In addition, the amino acid sequences of the CDRs of both the heavy and light chains of the antibodies described herein are presented. Table 1 below correlates the sequence IDs to their respective sequence.

Table 1: Sequences of Anti-APRIL Antibodies SEQ Description Sequence IDNO 1 anti-hAPRIL GYTFTSYV heavy chain CDR1 (AA)

TABLE 1
Sequences of Anti-APRIL Antibodies

| SEQ | | |
|---|---|---|
| ID NO | Description | Sequence |
| 1 | anti-hAPRIL | GYTFTSYV |
| | heavy chain CDR1 | |
| | (AA) | |
| 2 | anti-hAPRIL | YINPYNDAPKYNEKFK |
| | heavy chain CDR2 | |
| | (AA) | |
| 3 | anti-hAPRIL | GLGYALYYAMDY |
| | heavy chain CDR3 | |
| | (AA) | |
| 4 | anti-hAPRIL light | KASQNVGNNVA |
| | chain CDR1 (AA) | |
| 5 | anti-hAPRIL light | SASNRDS |
| | chain CDR2 (AA) | |
| 6 | anti-hAPRIL light | QQYNIYPFTF |
| | chain CDR3 (AA) | |
| 7 | VH11 heavy chain | caggtccagc ttgtgcagtc tggggctgag gtgaagaagc ctggggcctc |
| | variable region | agtgaaggtt tcctgcaagg cttctggata cacattcact agctatgtga tgcattgggt |
| | (DNA) | gcgccaggcc cccggacaaa ggcttgagtg gatgggatat attaatcctt ataatgatgc |
| | | tcctaaatac aatgagaagt tcaaaggcag agtcaccatt accagggaca |
| | | catccgcgag cacagcctac atggagctga gcagcctgag atctgaagac |
| | | acggctgtgt attactgtgc gagaggcttg |
| | | ggttacgccc tttactatgc tatggactac tggggccaag ggaccacggt caccgtctcc |
| | | tca |
| 8 | VH11 heavy chain | QVQLVQSGAEVKKPGASVKVSCKASGYTFTSYVMHWVRQ |
| | variable region | APGQRLEWMGYINPYNDAPKYNEKFKGRVTITRDTSASTA |
| | (AA) | YMELSSLRSEDTAVYYCARGLGYALYYAMDYWGQGTTV |
| | | TVSS |
| 9 | VH12 heavy chain | caggtccagc ttgtgcagtc tggggctgag gtgaagaagc ctggggcctc |
| | variable region | agtgaaggtc tcctgcaagg cttctggata cacattcact agctatgtga tgcactgggt |
| | (DNA) | gcgacaggcc cctggacaag ggcttgagtg gatgggatat attaatcctt ataatgatgc |
| | | tcctaaatac aatgagaagt tcaaaggcag ggtcaccatg accagggaca |
| | | cgtccatcag cacagcctac atggagctga gcaggctgag atctgacgac |
| | | acggccgtgt attactgtgc gagaggcttg |
| | | ggttacgccc tttactatgc tatggactac tggggccaag ggaccacggt caccgtctcg |
| | | agc |
| 10 | VH12 heavy chain | QVQLVQSGAEVKKPGASVKVSCKASGYTFTSYVMHWVRQ |
| | variable region | APGQGLEWMGYINPYNDAPKYNEKFKGRVTMTRDTSISTA |
| | (AA) | YMELSRLRSDDTAVYYCARGLGYALYYAMDYWGQGTTV |
| | | TVSS |
| 11 | VH13 heavy chain | caggtccagc ttgtgcagtc tggggctgag gtgaagaagc ctggggcctc |
| | variable region | agtgaaggtt tcctgcaagg catctggata cacattcact agctatgtga tgcactgggt |
| | (DNA) | gcgacaggcc cctggacaag ggcttgagtg gatgggatat attaatcctt ataatgatgc |
| | | tcctaaatac aatgagaagt tcaaaggcag agtcaccatg accagggaca |
| | | cgtccacgag cacagtctac atggagctga gcagcctgag atctgaggac |
| | | acggccgtgt attactgtgc gagaggcttg ggttacgccc tttactatgc tatggactac |
| | | tggggccaag ggacaatggt caccgtctcgagc |
| 12 | VH13 heavy chain | QVQLVQSGAEVKKPGASVKVSCKASGYTFTSYVMHWVRQ |
| | variable region | APGQGLEWMGYINPYNDAPKYNEKFKGRVTMTRDTSTST |
| | (AA) | VYMELSSLRSEDTAVYYCARGLGYALYYAMDYWGQGTM |
| | | VTVSS |
| 13 | VH14 heavy chain | caggtccagc ttgtgcagtc tggggctgag gtgaagaagc ccggcgccag |
| | variable region | cgtgaaggtg agctgcaagg ccagcggata cacattcact agctatgtga |
| | (DNA) | tgcactgggt gagacaggcc cccggccagg gcctggagtg gatgggctat |
| | | attaatcctt ataatgatgc tcctaaatac aatgagaagt tcaaaggcag agtgaccatg |
| | | accagagaca ccagcgccag caccgcctac atggagctga gcagcctgag |
| | | aagcgacgac accgccgtgt actactgcgc cagaggcttg |
| | | ggttacgccc tttactatgc tatggactac tggggccagg gcaccaccgt gaccgtgagc |
| | | agc |
| 14 | VH14 heavy chain | QVQLVQSGAEVKKPGASVKVSCKASGYTFTSYVMHWVRQ |
| | variable region | APGQGLEWMGYINPYNDAPKYNEKFKGRVTMTRDTSAST |
| | (AA) | AYMELSSLRSDDTAVYYCARGLGYALYYAMDYWGQGTT |
| | | VTVSS |
| 15 | VH14_1 heavy | caggtccagc ttgtgcagtc tggggctgag gtgaagaagc ccggcgccag |
| | chain variable | cgtgaaggtg agctgcaagg ccagcggata cacattcact agctatgtga |
| | region (DNA) | tgcactgggt gagacaggcc cccggccagg gcctggagtg gatgggctat |
| | | attaatcctt ataatgatgc tcctaaatac aatgagaagt tcaaaggcag agtgaccatg |
| | | accagtgaca ccagcgccag caccgcctac atggagctga gcagcctgag |
| | | aagcgacgac accgccgtgt actactgcgc cagaggcttg |
| | | ggttacgccc tttactatgc tatggactac tggggccagg gcaccaccgt gaccgtgagc |
| | | agc |
| 16 | VH14_1 heavy | QVQLVQSGAEVKKPGASVKVSCKASGYTFTSYVMHWVRQ |
| | chain variable | APGQGLEWMGYINPYNDAPKYNEKFKGRVTMTSDTSAST |
| | region (AA) | AYMELSSLRSDDTAVYYCARGLGYALYYAMDYWGQGTT |
| | | VTVSS |
| 17 | VH14_1C heavy | caggtccagc ttgtgcagtc tggggctgag gtgaagaagc ccggcgccag |
| | chain variable | cgtgaaggtg agctgcaagg ccagcggata cacattcact agctatgtga tgcactgggt |
| | region (DNA) | gagacaggcc cccggccagg gcctggagtg gatgggctat attaatcctt |
| | | ataatgatgc tcctaaatac aatgagaagt tcaaaggcag agtgaccatc accagtgaca |
| | | ccagcgccag caccgcctac atggagctga gcagcctgag aagcgacgac |
| | | accgccgtgt actactgcgc cagaggcttg |
| | | ggttacgccc tttactatgc tatggactac tggggccagg gcaccaccgt gaccgtgagc |
| | | agc |
| 18 | VH14_1C heavy | QVQLVQSGAEVKKPGASVKVSCKASGYTFTSYVMHWVRQ |
| | chain variable | APGQGLEWMGYINPYNDAPKYNEKFKGRVTITSDTSASTA |
| | region (AA) | YMELSSLRSDDTAVYYCARGLGYALYYAMDYWGQGTTV |
| | | TVSS |
| 19 | VH14_1D heavy | caggtccagc ttgtgcagtc tggggctgag gtgaagaagc ccggcgccag |
| | chain variable | cgtgaaggtg agctgcaagg ccagcggata cacattcact agctatgtga |
| | region (DNA) | tgcactgggt gagacaggcc cccggccagg gcctggagtg gatgggctat |
| | | attaatcctt ataatgatgc tcctaaatac aatgagaagt tcaaaggcag agtgaccatg |
| | | accagtgaca agagcgccag caccgcctac atggagctga gcagcctgag |
| | | aagcgacgac accgccgtgt actactgcgc cagaggcttg |
| | | ggttacgccc tttactatgc tatggactac tggggccagg gcaccaccgt gaccgtgagc |
| | | agc |
| 20 | VH14_1D heavy | QVQLVQSGAEVKKPGASVKVSCKASGYTFTSYVMHWVRQ |
| | chain variable | APGQGLEWMGYINPYNDAPKYNEKFKGRVTMTSDKSAST |
| | region (AA) | AYMELSSLRSDDTAVYYCARGLGYALYYAMDYWGQGTT |
| | | VTVSS |
| 21 | VH14_1E heavy | gaggtccagc ttgtgcagtc tggggctgag gtgaagaagc ccggcgccag |
| | chain variable | cgtgaaggtg agctgcaagg ccagcggata cacattcact agctatgtga |
| | region (DNA) | tgcactgggt gagacaggcc cccggccagg gcctggagtg gatgggctat |
| | | attaatcctt ataatgatgc tcctaaatac aatgagaagt tcaaaggcag agtgaccatg |
| | | accagtgaca ccagcgccag caccgcctac atggagctga gcagcctgag |
| | | aagcgacgac accgccgtgt actactgcgc cagaggcttg |
| | | ggttacgccc tttactatgc tatggactac tggggccagg gcaccaccgt gaccgtgagc |
| | | agc |
| 22 | VH14_1E heavy | EVQLVQSGAEVKKPGASVKVSCKASGYTFTSYVMHWVRQ |
| | chain variable | APGQGLEWMGYINPYNDAPKYNEKFKGRVTMTSDTSAST |
| | region (AA) | AYMELSSLRSDDTAVYYCARGLGYALYYAMDYWGQGTT |
| | | VTVSS |
| 23 | VH14_1G heavy | caggtccagc ttgtgcagtc tggggctgag gtgaagaagc ccggcgccag |
| | chain variable | cgtgaaggtg agctgcaagg ccagcggata cacattcact agctatgtga |
| | region (DNA) | tgcactgggt gagacaggcc cccggccagg gcctggagtg gatgggctat |
| | | attaatcctt ataatgatgc tcctaaatac aatgagaagt tcaaaggcaa agcgaccatg |
| | | accagtgaca ccagcgccag caccgcctac atggagctga gcagcctgag |
| | | aagcgacgac accgccgtgt actactgcgc cagaggcttg |
| | | ggttacgccc tttactatgc tatggactac tggggccagg gcaccaccgt gaccgtgagc |
| | | agc |
| 24 | VH14_1G heavy | QVQLVQSGAEVKKPGASVKVSCKASGYTFTSYVMHWVRQ |
| | chain variable | APGQGLEWMGYINPYNDAPKYNEKFKGKATMTSDTSAST |
| | region (AA) | AYMELSSLRSDDTAVYYCARGLGYALYYAMDYWGQGTT |
| | | VTVSS |
| 25 | VL15 light chain | gacatcgtga tgacccagtc tccttccacc ctgtctgcat ctgtaggaga cagagtcacc |
| | variable region | atcacttgca aggccagtca gaatgtgggt aataatgtag cctggtatca gcagaaacca |
| | (DNA) | gggaaagccc ctaagctcct gatctcttcg gcatccaacc gtgacagtgg ggtcccatca |
| | | aggttcagcg gcagtggatc tgggacagag ttcactctca ccatcagcag cctgcagcct |
| | | gatgattttg caacttattt ctgccagcaa tataacatct atccattcac gtttggccag |
| | | gggaccaagc tggagatcaa a |
| 26 | VL15 light chain | DIVMTQSPSTLSASVGDRVTITCKASQNVGNNVAWYQQKP |
| | variable region | GKAPKLLISSASNRDSGVPSRFSGSGSGTEFTLTISSLQPDDF |
| | (AA) | ATYFCQQYNIYPFTFGQGTKLEIK |
| 27 | VH14_1G heavy | caggtccaac ttgtgcagtc tggggctgaa gtgaagaagc ccggcgctag |
| | chain (DNA) | tgtgaaggtt tcatgtaagg cttctggata cacatttact tcatatgtaa tgcactgggt |
| | | gcgtcaagcc cctggccagg gcctggagtg gatggggtat attaatcctt ataatgatgc |
| | | tcctaaatac aatgagaagt tcaaaggaaa agcaactatg accagtgata ctagcgcttc |
| | | aaccgcctac atggagctga gcagcttaag aagcgacgac accgccgtgt |
| | | actattgtgc caggggcttg ggttacgccc tttattatgc tatggactac tggggtcagg |
| | | gcaccacagt gaccgttagc |
| | | tctgcatcta ctaagggacc atccgtcttc cccctggcgc catgctcccg cagtacaagt |
| | | gagagcacag cagccctggg ctgtttggta aaggactact tccccgaacc tgtgactgtg |
| | | tcttggaact caggcgccct gactagcggc gtgcacactt tccctgctgt cctacagtcc |
| | | tcaggactat actccctctc gtctgtggtg acagtgcctt cctcatcatt gggaacgaaa |
| | | acctatactt gcaacgttga tcacaagccc agcaacacca aggtggacaa |
| | | gagagttgag |
| | | tccaaatatg gtcccccatg tccaccatgt ccagcacctg agtttcttgg cggaccaagt |
| | | gttttcctgt tccccccaaa acccaaggat actctcatga taagtcgcac ccctgaagtc |
| | | acttgcgtgg tggtggacgt tagccaggaa gatcccgaag tccaattcaa ctggtacgta |
| | | gatggcgtag aagtgcataa tgcgaagaca aagccgagag aggagcagtt |
| | | taattcgacg tatcgggtgg tcagcgtcct cacagtcctg caccaggact ggctgaacgg |
| | | caaggagtat aagtgcaagg tctccaacaa aggtctcccg tcctccattg agaaaacaat |
| | | ctccaaagca |
| | | aaagggcagc cccgagaacc acaagtgtac accctgcccc catctcagga |
| | | ggagatgacc aagaaccagg tcagtcttac ctgcctggtc aaaggctttt atccctcaga |
| | | tatcgccgtt gagtgggaaa gcaatgggca gccggagaac aactacaaga |
| | | ccacgcctcc cgttctggat tctgacggat cgttcttttt atacagcagg ctaaccgtgg |
| | | acaagtctcg gtggcaggaa |
| | | gggaatgtat tttcttgcag tgtaatgcat gaggctctgc acaatcatta cacacagaag |
| | | tctctctccc tgtctcttgg taaa |
| 28 | VH14_1G heavy | QVQLVQSGAEVKKPGASVKVSCKASGYTFTSYVMHWVRQ |
| | chain (AA) | APGQGLEWMGYINPYNDAPKYNEKFKGKATMTSDTSAST |
| | | AYMELSSLRSDDTAVYYCARGLGYALYYAMDYWGQGTT |
| | | VTVSSASTKGPSVFPLAPCSRSTSESTAALGCLVKDYFPEPV |
| | | TVSWNSGALTSGVHTFPAVLQSSGLYSLSSVVTVPSSSLGT |
| | | KTYTCNVDHKPSNTKVDKRVESKYGPPCPPCPAPEFLGGPS |
| | | VFLFPPKPKDTLMISRTPEVTCVVVDVSQEDPEVQFNWYV |
| | | DGVEVHNAKTKPREEQFNSTYRVVSVLTVLHQDWLNGKE |
| | | YKCKVSNKGLPSSIEKTISKAKGQPREPQVYTLPPSQEEMT |
| | | KNQVSLTCLVKGFYPSDIAVEWESNGQPENNYKTTPPVLD |
| | | SDGSFFLYSRLTVDKSRWQEGNVFSCSVMHEALHNHYTQK |
| | | SLSLSLGK |
| 29 | VL15 light chain | gacattgtga tgacccagtc tccttccact ctctctgcat ctgtgggaga cagagtaacc |
| | (DNA) | atcacttgca aggccagtca gaatgtgggt aataatgtag cctggtatca gcagaaacca |
| | | gggaaagccc ctaagctact gatctcttcc gcatccaacc gggacagtgg tgtgccctca |
| | | aggtttagcg gcagtggatc agggacagag ttcacattga ccatatccag cctgcagcct |
| | | gatgattttg ctacttattt ctgccaacaa tataacattt acccattcac gtttggccag |
| | | ggcaccaagc tagagatcaa acggacggtt gctgcaccct ctgtctttat cttcccgcca |
| | | tctgatgaac agttgaagtc cggaacagcc tctgttgtgt gcctgctgaa taacttttat |
| | | ccccgcgagg cgaaagttca gtggaaggtg gataacgccc tccaatcagg |
| | | caattcccag |
| | | gagagtgtga cagagcaaga ttccaaggac tcaacctaca gcctcagcag tactttaact |
| | | ctgagcaaag cagactacga gaagcacaaa gtctacgctt gcgaagtcac |
| | | ccatcagggc cttagctcgc ccgtcacaaa gagctttaac aggggagaat gt |
| 30 | VL15 light chain | DIVMTQSPSTLSASVGDRVTITCKASQNVGNNVAWYQQKP |
| | (AA) | GKAPKLLISSASNRDSGVPSRFSGSGSGTEFTLTISSLQPDDF |
| | | ATYFCQQYNIYPFTFGQGTKLEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 31 | Heavy chain | atggactgga cctggaggat cctctttttg gtggcagcag ccacaggtgc ccactcc |
| | secretion leader | |
| | sequence (DNA) | |
| 32 | Heavy chain | MDWTWRILFLVAAATGAHS |
| | secretion leader | |
| | sequence (AA) | |
| 33 | Light chain | atggacatga gagtcctcgc tcagctcctg gggctcctgc tgctctgttt cccaggtgcc |
| | secretion leader | agatgt |
| | sequence (DNA) | |
| 34 | Light chain | MDMRVLAQLLGLLLLC |
| | secretion leader | |
| | sequence (AA) | |

Anti-APRIL antibodies have been described previously in U.S. Appl. Publ. No. 2021/0379183.

Various dosing schedules may be employed as described hereinafter. In certain embodiments, the method comprises repeating the infusion or subcutaneous administration on a weekly (“QW”) schedule for multiple cycles (e.g., 4 weeks, 6 weeks, 8 weeks, etc.). In other embodiments, the method comprises repeating the infusion or subcutaneous administration on a schedule of at least every two weeks (“biweekly as used herein” or “Q2W”) schedule for multiple cycles (e.g., 4 weeks, 6 weeks, 8 weeks, etc.). Alternatively, the method comprises repeating the infusion or subcutaneous administration on a schedule of at least every 4 weeks (“Q4W”) or once per month (“QMT”) schedule for multiple cycles (e.g., 8 weeks, 12 weeks, 16 weeks, etc.). In certain embodiments, a frontloading dosing schedule is used. The term “frontloading” when referring to active agent administration refers to the initial loading dose, followed by the maintenance dose. The initial loading dose (single or multiple) is intended to more quickly increase the serum active agent concentration of an animal or human patient to an effective target serum concentration. In various embodiments, frontloading is accomplished by initial dosing delivered over 3 weeks or less so that the antibody reaches the target serum concentration. Preferably, the loading dose or series of doses is administered for 2 weeks or less, more preferably 1 week or less, e.g., I day or less. Most preferably, the loading dosing is a single dosing, with no maintenance dosing thereafter for at least one week, and the loading dosing is administered in 1 day or less. In order to avoid adverse immune reactions to antibody active agents, it may be preferred to deliver the loading dose of antibody is administered by intravenous injection. The present disclosure includes loading and maintenance doses of frontloading active agent delivery by intravenous or subcutaneous administration.

Administration of the loading dose can be, for example, one or more dosings at a time interval of at least about 1, 2, 3, 4, 5, 6, 7 or 8 weeks apart. In some embodiments, the at least one loading dose is administered by one or more intravenous injections and then at least one maintenance dose by one or more intravenous or subcutaneous administrations. In other embodiments, the instructions can be for administering at least one loading dose by, for example, one or more intravenous or subcutaneous administrations and at least one maintenance dose by one or more intravenous or subcutaneous administrations. In certain embodiments, both the at least one loading dose as well as the at least one maintenance dose is administered subcutaneously. In other embodiments, the at least one loading dose is administered by intravenous infusion followed by at least one maintenance dose administered subcutaneously. For example, the method of treatment can comprise administering a loading dose of 150-1350 mg of the APRIL binding antibody or antigen-binding fragment thereof, e.g., BION-1301, by intravenous infusion or subcutaneous injection. After the loading dose (e.g., 1 week, 2 weeks, 3 weeks or 4 weeks after the loading dose), a maintenance dose of 600 mg or less of the APRIL binding antibody or antigen-binding fragment thereof, e.g., BION-1301, can be administered every 4 weeks or less, preferably every 3 weeks or less, more preferably every 2 weeks or less, and in embodiments every 1 week or less, by subcutaneous injection.

A loading dose of an active agent can be larger (e.g., about 1.5, 2, 3, 4 or 5 times larger) than a subsequent maintenance dose. The one or more therapeutically effective maintenance doses can be any therapeutically effective amount described herein. The loading dose can be about 2 or 3 times larger than the maintenance dose. Active agent can be administered in two (or more) loading doses prior to the maintenance dose. A first loading dose of the antibody or fragment thereof can be administered on day 1, a second loading dose can be administered, e.g., about 1 or 2 weeks later, and a maintenance dose can be administered, e.g., once weekly or once every 2 weeks thereafter for the duration of treatment. The first loading dose can be about 3 or 4 times larger than the maintenance dose, and the second loading dose can be about 2, 3, 4, 5, or more times larger than the maintenance dose.

In one example a loading dosing schedule comprising administration, either by intravenous infusion or subcutaneous administration, repeating at least every two weeks for up to at least 4 weeks, is followed by a maintenance dosing schedule comprising administration, either by intravenous infusion or subcutaneous administration, wherein the maintenance dosing schedule results in administration of less of the anti-APRIL antibody, either by each administration comprising less anti-APRIL antibody, or by administering at longer intervals than during the loading dosing schedule. In another example, a loading dosing schedule comprising administration, either by intravenous infusion or subcutaneous administration, repeating at least daily and more preferably twice daily for up to at least 4 days, is followed by a maintenance dosing schedule comprising administration, either by intravenous infusion or subcutaneous administration, such as on a QW, Q2W, Q4W, QM, etc. schedule. In one embodiment, the loading dosing schedule comprises administering the antibody by intravenous infusion, and the maintenance dosing schedule comprises administering the antibody by subcutaneous injection. In another embodiment, both the loading dosing schedule and the maintenance dosing schedule comprises administering the antibody by subcutaneous injection. In another embodiment, both the loading dosing schedule and the maintenance dosing schedule comprises administering the antibody by intravenous infusion. This is not meant to be an exhaustive list of dosing schedules.

By way of example only, the subcutaneous injection of the method comprises administering about 2 mL of the antibody formulation into the patient's preferred injection site (e.g., thigh, abdomen, upper arm, etc.). In preferred embodiments, the anti-APRIL antibody of the formulation is at a concentration of about 150 mg/mL, resulting in administration of about 300 mg of anti-APRIL antibody in a single injection. In certain embodiments, the subcutaneous injection of the method comprises administering about 4 mL (as a single injection or as 2×2 mL injections) of the antibody formulation of the anti-APRIL antibody at a concentration of about 150 mg/mL, resulting in administration of about 600 mg of anti-APRIL antibody. The volume of administration, and the number of injections required as part of a single administration, may be adjusted as necessary to achieve a total desired dose of between about 10 mg to about 1350 mg of the anti-APRIL antibody.

In certain other embodiments, the intravenous infusion of the method comprises: (a) diluting the formulation of the first aspect of the disclosure, and embodiments thereof, to a concentration of between about 0.1 mg/mL to about 10 mg/mL in 0.9% saline; and (b) administering a total dose of between about 10 mg to about 1350 mg of the anti-APRIL antibody to the individual in a single intravenous dose of the diluted formulation over a period of about 2 hours. Again, by way of example only, about 15 mL of a formulation at an anti-APRIL antibody concentration of about 20 mg/mL is added to about 235 mL of 0.9% saline to provide the intravenous dose at a concentration of about 1.2 mg/mL.

In certain embodiments, the method of administering an anti-APRIL antibody to an individual in need thereof comprises administering the formulation described herein by a loading/maintenance administration protocol. Such a protocol may comprise a loading component of the protocol that comprises one or more administrations of the anti-APRIL antibody at a higher concentration than the anti-APRIL antibody concentration in the maintenance component of the loading/maintenance administration protocol; one or more administrations of the anti-APRIL antibody at a higher frequency than the frequency of administration of the anti-APRIL antibody in the maintenance component of the loading/maintenance administration protocol; and/or one or more administrations of the anti-APRIL antibody at a different route than the route of administration of the anti-APRIL antibody in the maintenance component of the loading/maintenance administration protocol.

By way of example only, the loading component of the loading/maintenance administration protocol may comprise one or more intravenous administrations of the anti-APRIL antibody and the maintenance component of the loading/maintenance administration protocol comprises one or more subcutaneous administrations of the anti-APRIL antibody. In such an example, the concentration of the loading administration(s) may be higher and/or the frequency of administration may be greater than is used in the maintenance administration(s).

In another example, the loading component of the loading/maintenance administration protocol may comprise one or more subcutaneous administrations of the anti-APRIL antibody and the maintenance component of the loading/maintenance administration protocol comprises one or more intravenous administrations of the anti-APRIL antibody. In such an example, the concentration of the loading administration(s) may be higher and/or the frequency of administration may be greater than is used in the maintenance administration(s).

In another example, the loading component of the loading/maintenance administration protocol may comprise one or more subcutaneous administrations of the anti-APRIL antibody and the maintenance component of the loading/maintenance administration protocol comprises one or more subcutaneous administrations of the anti-APRIL antibody. In such an example, the concentration of the loading administration(s) may be higher and/or the frequency of administration may be greater than is used in the maintenance administration(s).

In one embodiment, the loading dose comprises intravenous infusion of 150 to 1350 mg of an anti-APRIL antibody, with at least one subsequent infusion of that amount at a first time interval, and the maintenance dose comprises administering either i) a lower amount of the anti-APRIL antibody administered at the first time interval after the last loading dose infusion, with at least one subsequent administration at the lower amount and the same time interval for at least 12 weeks, ii) the same amount of anti-APRIL antibody administered at a second time interval after the last loading dose infusion, with at least one subsequent administration at the same amount and the second time interval for at least 12 weeks, wherein the second time interval is longer than the first time interval, or iii) a lower amount of the anti-APRIL antibody administered at the second time interval after the last loading dose infusion, with at least one subsequent administration the same amount at the second time interval for a least 12 weeks, wherein the maintenance dosing may be by intravenous infusion or by subcutaneous injection, preferably subcutaneous injection. In one embodiment, the loading dose comprises subcutaneous injection of 150 to 1350 mg of an anti-APRIL antibody, with at least one subsequent subcutaneous injection of that amount at a first time interval, and the maintenance dose comprises administering either i) a lower amount of the anti-APRIL antibody administered at the first time interval after the last loading dose infusion, with at least one subsequent administration at the lower amount and the same time interval for at least 12 weeks, ii) the same amount of anti-APRIL antibody administered at a second time interval after the last loading dose infusion, with at least one subsequent administration at the same amount and the second time interval for at least 12 weeks, wherein the second time interval is longer than the first time interval, or iii) a lower amount of the anti-APRIL antibody administered at the second time interval after the last loading dose infusion, with at least one subsequent administration the same amount at the second time interval for a least 12 weeks, wherein the maintenance dosing may be by intravenous infusion or by subcutaneous injection.

In certain embodiments, the subject has been determined to have controlled serum glucose levels. In some embodiments, the subject with controlled serum glucose levels is not being treated for diabetes. In some embodiments, the subject with controlled serum glucose levels is being treated for diabetes. In some embodiments, the subject with controlled serum glucose levels is not being treated for Type 2 diabetes. In some embodiments, the subject with controlled serum glucose levels is being treated for Type 2 diabetes. In some embodiments, the subject has been determined to have controlled serum glucose levels. For example, the subject has been determined to have a fasting serum glucose level of below about 130 mg/dL, about 125 mg/dL, about 120 mg/dL, about 115 mg/dL, about 110 mg/dL, about 105 mg/dL, about 100 mg/dL, about 95 mg/dL, about 90 mg/dL, about 85 mg/dL, about 80 mg/dL, or about 75 mg/dL, or any value in between.

In another aspect, provided herein is a method of inhibiting mesangial cell activation in a subject having IgA nephropathy, comprising administering to the subject an endothelin receptor antagonist, or a pharmaceutically acceptable salt thereof, and the APRIL binding antibody or antigen-binding fragment thereof. In some instances, the inhibiting of mesangial cell activation comprises reducing mesangial cell inflammation and/or activity of one or more biomarkers indicative of mesangial cell proliferation. Reducing mesangial cell inflammation includes reducing expression and/or activity of one or more of 1L6, MCP1 or other biomarkers indicative of mesangial cell inflammation. In some instances, the inhibiting of mesangial cell activation comprises reducing the pro-fibrotic response in the mesangial cells. In some instances, the mesangial activation is induced by IgA immune complexes. In some instances, the mesangial activation is associated with the presence of IgA immune complexes.

In some embodiments, the renal inflammation in the subject having IgA nephropathy is decreased by at least about 10% after treatment with an endothelin receptor antagonist, or a pharmaceutically acceptable salt thereof, or a pharmaceutically acceptable salt thereof, and an APRIL binding antibody or an antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, the renal inflammation in the subject is decreased by at least about 20%, about 30%, about 40%, about 50%, about 60%, about 70%, about 80%, about 90%, or about 95%, or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In some embodiments, renal fibrosis in the subject having IgA nephropathy is decreased by at least about 10% after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, the renal fibrosis in the subject is decreased by at least about 20%, about 30%, about 40%, about 50%, about 60%, about 70%, about 80%, about 90%, or about 95%, or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In some embodiments, renal fibrosis in the subject having IgA nephropathy is decreased to less than about 50% of the cortical area of the affected kidney(s) after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, about renal fibrosis in the subject is decreased to less than about 40% of the cortical area. For example, in some embodiments, renal fibrosis in the subject is decreased to less than about 35%, about 30%, about 25%, about 20%, about 15%, or about 10%, or any value in between, of the cortical area. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In another aspect, provided herein is a method of decreasing the occurrence of hematuria in a subject having IgA nephropathy, comprising administering an endothelin receptor antagonist, or a pharmaceutically acceptable salt thereof, and an APRIL binding antibody or an antigen-binding fragment thereof, each as described herein.

In some embodiments, the number of urinary red blood cells per high powered (microscope) field (rbc/hpf) in the subject having IgA nephropathy is decreased by at least about 10% after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, the urinary rbc/hpf in the subject is decreased by at least about 20%, about 30%, about 40%, about 50%, about 60%, about 70%, about 80%, about 90%, or about 95%, or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In another aspect, provided herein is a method of stabilizing eGFR, in a subject having IgA nephropathy comprising administering an endothelin receptor antagonist, or a pharmaceutically acceptable salt thereof, and the APRIL binding antibody or antigen-binding fragment thereof.

In some embodiments, provided herein is a method for reducing the rate of decrease of eGFR in a subject having IgA nephropathy, the method comprising administering an endothelin receptor antagonist, or a pharmaceutically acceptable salt thereof, and the APRIL binding antibody or antigen-binding fragment thereof. In some embodiments, the rate of decrease of eGFR of the subject is reduced by at least about 10% after treatment with an endothelin receptor antagonist, or a pharmaceutically acceptable salt thereof, and the APRIL binding antibody or antigen-binding fragment thereof (e.g., after treatment for 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In some embodiments, the rate of decrease of eGFR of the subject is reduced by at least about 20%; by at least about 30%; by at least about 40%: by at least about 50%; by at least about 60%; by at least about 70%; by at least about 80%; by at least about 90%; or by at least about 95%; or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days. In certain of the foregoing embodiments, the subject has been treated with an endothelin receptor antagonist, or a pharmaceutically acceptable salt thereof, and the APRIL binding antibody or antigen-binding fragment thereof for between about 6 months and about 1 year.

In some embodiments, the rate of decrease of eGFR of the subject having IgA nephropathy is reduced to below about 10 mL/min/1.73 m² after treatment with an endothelin receptor antagonist, or a pharmaceutically acceptable salt thereof, and the APRIL binding antibody or antigen-binding fragment thereof. For example, after treatment for 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between. In certain embodiments, the rate of decrease of eGFR of the subject is reduced to below about 9 mL/min/1.73 m², about 8 mL/min/1.73 m², about 7 mL/min/1.73 m², about 6 mL/min/1.73 m², about 5 mL/min/1.73 m², about 4 mL/min/1.73 m², about 3 mL/min/1.73 m², about 2 mL/min/1.73 m², about 1 mL/min/1.73 m², or about 0.75 mL/min/1.73 m², or any value in between after treatment with an endothelin receptor antagonist, or a pharmaceutically acceptable salt thereof, and the APRIL binding antibody or antigen-binding fragment thereof for between about 6 months to about 1 year. The typical decline in eGFR with age, for example, in a subject between about 20 to about 30 years of age, is about 1 mL/min/1.73 m² per year.

In another aspect, provided herein is method of decreasing the number of IgA-nephropathy associated disease flares in a subject having IgA nephropathy, comprising administering an endothelin receptor antagonist, or a pharmaceutically acceptable salt thereof, and the APRIL binding antibody or antigen-binding fragment thereof to a subject in need thereof. In some embodiments, the method decreases disease flares associated with hematuria.

In some embodiments, the method decreases disease flares associated with proteinuria. In some embodiments, the method decreases IgA-nephropathy associated disease flares which are associated with systemic manifestations. In some embodiments, the method decreases declines in eGFR as described anywhere herein. In some embodiments, the method decreases one or more of edema, fatigue, hematuria, or gross hematuria. In some embodiments, the method positively impacts disease progression.

In another aspect, provided herein is a method of delaying the onset of ESRD in a subject having IgA nephropathy, comprising administering an endothelin receptor antagonist, or a pharmaceutically acceptable salt thereof, and the APRIL binding antibody or antigen-binding fragment thereof to a subject in need thereof.

In some embodiments, the method increases the time between the diagnosis of IgA nephropathy in the subject and the time when eGFR of the subject falls below 15 mL/min/1.73 m². In certain embodiments, the method increases the time between the diagnosis of IgA nephropathy in the subject and the time when eGFR of the subject falls below 15 mL/min/1.73 m² by at least about 10%. For example, in some embodiments, the method increases the time between the diagnosis of IgA nephropathy in the subject and the time when eGFR of the subject falls below 15 mL/min/1.73 m² by at least about 20%, about 30%, about 40%, about 50%, about 60%, about 70%, about 80%, about 90%, about 100%, about 150%, about 200%, about 250%, about 300%, about 350%, about 400%, about 450%, or about 500%, or any value in between.

In certain embodiments, the method increases the time between the diagnosis of IgA nephropathy in the subject and the time when eGFR of the subject falls below 15 mL/min/1.73 m² by at least about 1 year. For example, the method can delay the time when eGFR of the subject falls below 15 mL/min/1.73 m² by at least about 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 11 years, 12 years, 13 years, 15 years, 15 years, 16 years, 17 years, 18 years, 19 years, or 20 years.

In another aspect, provided herein is a method of decreasing proteinuria in a subject having IgA nephropathy, comprising administering an endothelin receptor antagonist, or a pharmaceutically acceptable salt thereof, and the APRIL binding antibody or antigen-binding fragment thereof to a subject in need thereof.

In some embodiments, the amount of proteins (e.g., albumin) in the urine of the subject having IgA nephropathy is reduced by at least about 10% after treatment with an endothelin receptor antagonist, or a pharmaceutically acceptable salt thereof, and the APRIL binding antibody or antigen-binding fragment thereof (e.g., after treatment for 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In some embodiments, the amount of proteins in the urine of the subject is reduced by at least about 15%, about 20%, about 25%, about 30%, about 35%, about 40%, about 50%, about 55%, about 60%, about 65%, about 70%, about 75%, about 80%, about 85%, about 90%, or about 95%, or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In certain embodiments, the amount of proteins (e.g., albumin) in the urine of the subject having IgA nephropathy is reduced by about 20% to about 80% after between about 2 days and about 30 days of treatment with an endothelin receptor antagonist, or a pharmaceutically acceptable salt thereof, and the APRIL binding antibody or antigen-binding fragment thereof. In certain embodiments, the amount of proteins (e.g., albumin) in the urine of the subject having IgA nephropathy is reduced by about 20% to about 80% after between about 15 days and about 30 days of treatment with an endothelin receptor antagonist, or a pharmaceutically acceptable salt thereof, and the APRIL binding antibody or antigen-binding fragment thereof. In certain of these embodiments, the amount of proteins in the urine of the subject is reduced by about 25% to about 80%. In certain of these embodiments, the amount of proteins in the urine of the subject is reduced by about 30% to about 80%. In certain of these embodiments, the amount of proteins in the urine of the subject is reduced by about 35% to about 80%. In certain of these embodiments, the amount of proteins in the urine of the subject is reduced by about 40% to about 80%. In certain of these embodiments, the amount of proteins in the urine of the subject is reduced by about 45% to about 80%. In certain of these embodiments, the amount of proteins in the urine of the subject is reduced by about 50% to about 80%. In the aforementioned embodiments, the reduction of the amount of proteins (e.g., albumin) in the urine of the subject having IgA nephropathy is relative to the amount of proteins (e.g., albumin) in the urine prior to initiation of treatment with an endothelin receptor antagonist, or a pharmaceutically acceptable salt thereof, and the APRIL binding antibody or antigen-binding fragment thereof.

In some embodiments, the amount of proteins (e.g., albumin) in the urine of the subject having IgA nephropathy is reduced by about 100 mg/dL to about 3,000 mg/dL after treatment with an endothelin receptor antagonist, or a pharmaceutically acceptable salt thereof, and the APRIL binding antibody or antigen-binding fragment thereof (e.g., after treatment for 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, the amount of proteins in the urine of the subject is reduced by about 100 mg/dL to about 2,500 mg/dL. In certain embodiments, the amount of proteins in the urine of the subject is reduced by about 100 mg/dL to about 2,000 mg/dL. In certain embodiments, the amount of proteins in the urine of the subject is reduced by about 100 mg/dL to about 1,500 mg/dL. In certain embodiments, the amount of proteins in the urine of the subject is reduced by about 100 mg/dL to about 1,000 mg/dL. In certain embodiments, the amount of proteins in the urine of the subject is reduced by about 100 mg/dL to about 500 mg/dL. In certain embodiments, the amount of proteins in the urine of the subject is reduced by about 100 mg/dL to about 400 mg/dL. In certain embodiments, the amount of proteins in the urine of the subject is reduced by about 100 mg/dL to about 300 mg/dL. In certain embodiments, the amount of proteins in the urine of the subject is reduced by about 100 mg/dL to about 200 mg/dL. In certain embodiments, the amount of proteins in the urine of the subject is reduced by about 500 mg/dL to about 2,500 mg/dL. In certain embodiments, the amount of proteins in the urine of the subject is reduced by about 500 mg/dL to about 2,000 mg/dL. In certain embodiments, the amount of proteins in the urine of the subject is reduced by about 500 mg/dL to about 1,500 mg/dL. In certain embodiments, the amount of proteins in the urine of the subject is reduced by about 500 mg/dL to about 1,000 mg/dL. In certain embodiments, the amount of proteins in the urine of the subject is reduced by about 500 mg/dL to about 900 mg/dL. In certain embodiments, the amount of proteins in the urine of the subject is reduced by about 500 mg/dL to about 800 mg/dL. In certain embodiments, the amount of proteins in the urine of the subject is reduced by about 600 mg/dL to about 900 mg/dL. In certain embodiments, the amount of proteins in the urine of the subject is reduced by about 700 mg/dL to about 900 mg/dL. In certain embodiments, the amount of proteins in the urine of the subject is reduced by about 1,000 mg/dL to about 2,000 mg/dL. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days. In the aforementioned embodiments, the reduction of the amount of proteins (e.g., albumin) in the urine of the subject having IgA nephropathy is relative to the amount of proteins (e.g., albumin) in the urine prior to initiation of treatment with an endothelin receptor antagonist, or a pharmaceutically acceptable salt thereof, and the APRIL binding antibody or antigen-binding fragment thereof.

In certain embodiments, the amount of proteins (e.g., albumin) in the urine of the subject having IgA nephropathy is reduced by about 100 mg/dL to about 500 mg/dL after between about 15 days and about 30 days of treatment with an endothelin receptor antagonist, or a pharmaceutically acceptable salt thereof, and the APRIL binding antibody or antigen-binding fragment thereof. In certain embodiments, the amount of proteins in the urine of the subject is reduced by about 200 mg/dL to about 500 mg/dL after between about 15 days and about 30 days of treatment with an endothelin receptor antagonist, or a pharmaceutically acceptable salt thereof, and the APRIL binding antibody or antigen-binding fragment thereof. In certain embodiments, the amount of proteins in the urine of the subject is reduced by about 300 mg/dL to about 500 mg/dL after between about 15 days and about 30 days of treatment with an endothelin receptor antagonist, or a pharmaceutically acceptable salt thereof, and the APRIL binding antibody or antigen-binding fragment thereof. In the aforementioned embodiments, the reduction of the amount of proteins (e.g., albumin) in the urine of the subject having IgA nephropathy is relative to the amount of proteins (e.g., albumin) in the urine prior to initiation of treatment with an endothelin receptor antagonist, or a pharmaceutically acceptable salt thereof, and the APRIL binding antibody or antigen-binding fragment thereof.

In certain embodiments, the amount of proteins (e.g., albumin) in the urine of the subject having IgA nephropathy is reduced by about 500 mg/dL to about 900 mg/dL after between about 15 days and about 30 days of treatment with an endothelin receptor antagonist, or a pharmaceutically acceptable salt thereof, and the APRIL binding antibody or antigen-binding fragment thereof. In certain embodiments, the amount of proteins in the urine of the subject is reduced by about 600 mg/dL to about 900 mg/dL after between about 15 days and about 30 days of treatment with an endothelin receptor antagonist, or a pharmaceutically acceptable salt thereof, and the APRIL binding antibody or antigen-binding fragment thereof. In certain embodiments, the amount of proteins in the urine of the subject is reduced by about 700 mg/dL to about 900 mg/dL after between about 15 days and about 30 days of treatment with an endothelin receptor antagonist, or a pharmaceutically acceptable salt thereof, and the APRIL binding antibody or antigen-binding fragment thereof. In the aforementioned embodiments, the reduction of the amount of proteins (e.g., albumin) in the urine of the subject having IgA nephropathy is relative to the amount of proteins (e.g., albumin) in the urine prior to initiation of treatment with an endothelin receptor antagonist, or a pharmaceutically acceptable salt thereof, and the APRIL binding antibody or antigen-binding fragment thereof.

In some embodiments, the subject having IgA nephropathy has a reduced level of proteins (e.g., albumin) in the urine of below about 1.0 gram/day after treatment with an endothelin receptor antagonist, or a pharmaceutically acceptable salt thereof, and the APRIL binding antibody or antigen-binding fragment thereof (e.g., after treatment for 1 week, 2 weeks, 3 weeks, 4 weeks, 5 weeks, 6 weeks, 7 weeks, 8 weeks, 9 weeks, 10 weeks, 20 weeks, 30 weeks, 40 weeks, 50 weeks, 60 weeks, 70 weeks. 80 weeks, 90 weeks, 100 weeks, 110 weeks. 120 weeks. 130 weeks, 140 weeks, 150 weeks, 160 weeks, 170 weeks, 180 weeks, 190 weeks, or 200 weeks). In certain embodiments, the subject has a reduced level of proteins in the urine of below about 0.9 gram/day. In certain embodiments, the subject has a reduced level of proteins in the urine of below about 0.8 gram/day. In certain embodiments, the subject has a reduced level of proteins in the urine of below about 0.7 gram/day. In certain embodiments, the subject has a reduced level of proteins in the urine of below about 0.6 gram/day. In certain embodiments, the subject has a reduced level of proteins in the urine of below about 0.5 gram/day. In certain embodiments, the subject has a reduced level of proteins in the urine of below about 0.4 gram/day. In certain embodiments, the subject has a reduced level of proteins in the urine of below about 0.3 gram/day. In certain embodiments, the subject has a reduced level of proteins in the urine of below about 0.2 gram/day. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days. In the aforementioned embodiments, the reduction of the amount of proteins (e.g., albumin) in the urine of the subject having IgA nephropathy is relative to the amount of proteins (e.g., albumin) in the urine prior to initiation of treatment with an endothelin receptor antagonist, or a pharmaceutically acceptable salt thereof, and the APRIL binding antibody or antigen-binding fragment thereof.

In another aspect, provided herein is a method of decreasing fatigue in a subject having IgA nephropathy, comprising administering an endothelin receptor antagonist, or a pharmaceutically acceptable salt thereof, and an APRIL binding antibody or an antigen-binding fragment thereof, to a subject in need thereof.

In some embodiments, the fatigue of the subject having IgA nephropathy is reduced by about 5% to about 80% after treatment with an endothelin receptor antagonist, or a pharmaceutically acceptable salt thereof, and the APRIL binding antibody or antigen-binding fragment thereof (e.g., after treatment for 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, the fatigue is reduced by about 10% to about 75%. In certain embodiments, the fatigue is reduced by about 10% to about 70%. In certain embodiments, the fatigue is reduced by about 10% to about 65%. In certain embodiments, the fatigue is reduced by about 10% to about 60%. In certain embodiments, the fatigue is reduced by about 10% to about 55%. In certain embodiments, the fatigue is reduced by about 10% to about 50%. In certain embodiments, the fatigue is reduced by about 10% to about 45%. In certain embodiments, the fatigue is reduced by about 10% to about 40%. In certain embodiments, the fatigue is reduced by about 10% to about 35%. In certain embodiments, the fatigue is reduced by about 10% to about 30%. In certain embodiments, the fatigue is reduced by about 10% to about 25%. In certain embodiments, the fatigue is reduced by about 10% to about 20%. In certain embodiments, the fatigue is reduced by about 10% to about 15%. In certain embodiments, the fatigue is reduced by about 20% to about 75%. In certain embodiments, the fatigue is reduced by about 20% to about 70%. In certain embodiments, the fatigue is reduced by about 20% to about 65%. In certain embodiments, the fatigue is reduced by about 20% to about 60%. In certain embodiments, the fatigue is reduced by about 20% to about 55%. In certain embodiments, the fatigue is reduced by about 20% to about 50%. In certain embodiments, the fatigue is reduced by about 20% to about 45%. In certain embodiments, the fatigue is reduced by about 20% to about 40%. In certain embodiments, the fatigue is reduced by about 20% to about 35%. In certain embodiments, the fatigue is reduced by about 20% to about 30%. In certain embodiments, the fatigue is reduced by about 30% to about 75%. In certain embodiments, the fatigue is reduced by about 30% to about 70%. In certain embodiments, the fatigue is reduced by about 30% to about 65%. In certain embodiments, the fatigue is reduced by about 30% to about 60%. In certain embodiments, the fatigue is reduced by about 30% to about 55%. In certain embodiments, the fatigue is reduced by about 30% to about 50%. In certain embodiments, the fatigue is reduced by about 30% to about 45%. In certain embodiments, the fatigue is reduced by about 30% to about 40%. In certain embodiments, the fatigue is reduced by about 40% to about 75%. In certain embodiments, the fatigue is reduced by about 40% to about 70%. In certain embodiments, the fatigue is reduced by about 40% to about 65%. In certain embodiments, the fatigue is reduced by about 40% to about 60%. In certain embodiments, the fatigue is reduced by about 40% to about 55%. In certain embodiments, the fatigue is reduced by about 40% to about 50%. In certain embodiments, the fatigue is reduced by about 50% to about 75%. In certain embodiments, the fatigue is reduced by about 50% to about 70%. In certain embodiments, the fatigue is reduced by about 50% to about 65%. In certain embodiments, the fatigue is reduced by about 50% to about 60%. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days. In certain embodiments, the decrease in fatigue comprises a decrease in the score on one or more of the Fatigue Severity Scale, the Chalder Fatigue Scale, the FACIT Fatigue Scale, the Brief Fatigue Inventory, the FACT-F Subscale, Global Vigor and Affect, the May and Kline Adjective Checklist, the Pearson-Byars Fatigue Feeling Checklist, the Rhoten Fatigue Scale, the Schedule of Fatigue and Anergia, the Visual Analog Scale, or the Checklist Individual Strength. In the aforementioned embodiments, the reduction of fatigue experienced by the subject having IgA nephropathy is relative to the fatigue experienced by the subject prior to initiation of treatment with an endothelin receptor antagonist, or a pharmaceutically acceptable salt thereof, and the APRIL binding antibody or antigen-binding fragment thereof. In some embodiments, the decrease in fatigue comprises a decrease in the score on the Brief Fatigue Inventory.

### Subject Selection

The subject having IgA nephropathy as described anywhere herein can be diagnosed using one or more methods known in the art. Non-limiting examples include: kidney biopsy, detecting galactose-deficient IgA (e.g., Gd-IgAQ1), detecting anti-glycan antibodies, detecting deposition of IgA-immune complexes in the kidney, or a combination of any of the foregoing. In some embodiments, the diagnosis of IgA nephropathy comprises detecting deposition of IgA-immune complexes in the kidney. In certain embodiments, the diagnosis of IgA nephropathy comprises a kidney biopsy. In certain embodiments, the diagnosis of IgA nephropathy comprises detecting galactose-deficient IgA. In certain embodiments, the diagnosis of IgA nephropathy comprises detecting anti-glycan antibodies (e.g., KM55). In certain embodiments, the diagnosis of IgA nephropathy comprises a kidney biopsy followed by detecting deposition of IgA-immune complexes in the kidney (for example, by light microscopy and/or immunofluorescence microscopy).

In some embodiments, the presence and/or level of a particular protein in a subject is determined prior to administration of an endothelin receptor antagonist, or a pharmaceutically acceptable salt thereof, and the APRIL binding antibody or antigen-binding fragment thereof. For example, serum levels of Gd-IgAQ1, serum levels of autoantibodies specific for Gd-IgAQ1, and/or serum and/or urine levels of IgAQ1-containing immune complexes. See, e.g., Knoppova, et al., Front. Immunol., Vol. 17, Art. 117 (2016). In some embodiments, the subject has Gd-IgA levels in the 90^{th} percentile or above prior to administration of an endothelin receptor antagonist, or a pharmaceutically acceptable salt thereof, and the APRIL binding antibody or antigen-binding fragment thereof. In some embodiments, the subject has Gd-IgA levels in the 95^{th} percentile or above prior to administration of an endothelin receptor antagonist, or a pharmaceutically acceptable salt thereof, and the APRIL binding antibody or antigen-binding fragment thereof. In some embodiments, the subject's Gd-IgA levels decrease to below the 90^{th} percentile after treatment with an endothelin receptor antagonist, or a pharmaceutically acceptable salt thereof, and the APRIL binding antibody or antigen-binding fragment thereof, for between about 6 months to 1 year.

In certain embodiments, the subject has mesangial cellularity in about ≥50% (e.g., about ≥60%, about ≥70%, or about ≥80%) of the glomeruli, wherein mesangial cellularity is defined as more than four mesangial cells in any mesangial area of a glomerulus. In certain embodiments, endocapillary hypercellularity is present in the subject, wherein endocapillary hypercellularity is defined as hypercellularity due to an increased number of cells within glomerular capillary lumina. In certain embodiments, segmental sclerosis is present in the subject, wherein segmental sclerosis is defined as adhesion or sclerosis (obliteration of capillary lumina by matrix) in part of but not the whole glomerular tuft. In certain embodiments, the subject has tubular atrophy/interstitial fibrosis in about ≥50% (e.g., about ≥60%, about ≥65%, about ≥70%, about 275%, or about ≥80%) of the cortical area, wherein tubular atrophy/interstitial fibrosis is defined as the estimated percentage of cortical area showing tubular atrophy or interstitial fibrosis. In certain embodiments, the subject has crescents present on the glomeruli. In certain of these embodiments, the subject has crescents present on below about 25% (e.g., below about 20%, about 15%, about 10%, or about 5%) of the glomeruli. In certain embodiments, the subject has a MEST-C score of M1; E1; S1; T1 or T2; and/or C0 or C1 under the Oxford MEST-C classification system. The Oxford MEST-C classification system is defined in Kidney International (2009) 76, 546-556 and Nature Reviews Nephrology (2017) 13, 385-386, (Also see: Kidney Research and Clinical Practice (2016) 35, 197-203; and IgA Nephropathy in Medscape (accessed Nov. 4, 2019)).

In some embodiments, the subject is at a high risk of progression to ESRD. In certain of these embodiments, the subject is excreting an average of about 1 gram or more of protein in the urine per day for at least about 3 months prior to the first administration of an endothelin receptor antagonist, or a pharmaceutically acceptable salt thereof, and the APRIL binding antibody or antigen-binding fragment thereof. In certain embodiments, the subject has an average eGFR 60 mL/min/1.73 m² (e.g., about ≤55, about ≤50, about ≤45, about 40, about ≤35) for at least about 3 months prior to the first administration of an endothelin receptor antagonist, or a pharmaceutically acceptable salt thereof, and the APRIL binding antibody or antigen-binding fragment thereof. In certain of these embodiments, the subject has eGFR >30 mL/min/1.73 m² prior to the first administration of an endothelin receptor antagonist, or a pharmaceutically acceptable salt thereof, and the APRIL binding antibody or antigen-binding fragment thereof.

In some embodiments, the subject is excreting an average of about 1 gram or more of protein in the urine per day for at least about 3 months (e.g., at least about 4 months, at least about 5 months, at least about 6 months, at least about 7 months, at least about 8 months, at least about 9 months, at least about 10 months, at least about 11 months, at least about a year, at least about 1.5 years, or at least about 2 years) prior to the first administration of an endothelin receptor antagonist, or a pharmaceutically acceptable salt thereof, and the APRIL binding antibody or antigen-binding fragment thereof. For example, the subject can be excreting an average of about 1.1 gram, 1.2 grams, 1.3 grams, 1.4 grams, 1.5 grams, 1.6 grams, 1.7 grams, 1.8 grams, 1.9 grams, 2.0 grams, 2.1 grams, 2.2 grams, 2.3 grams, 2.4 grams, 2.5 grams, 2.6 grams, 2.7 grams. 2.8 grams, 2.9 grams, 3.0 grams, 3.1 grams, 3.2 grams, 3.3 grams, 3.4 grams, 3.5 grams, 5 grams, or 7.5 grams, or 10 grams, or any value in between, of protein in the urine per day for at least 3 months prior to the first administration of an endothelin receptor antagonist, or a pharmaceutically acceptable salt thereof, and the APRIL binding antibody or antigen-binding fragment thereof.

In some embodiments, the subject is excreting an average of from about 0.3 grams to about 2 grams of protein in the urine per day for at least about 3 months (e.g., at least about 4 months, at least about 5 months, at least about 6 months, at least about 7 months, at least about 8 months, at least about 9 months, at least about 10 months, at least about 11 months, at least about a year, at least about 1.5 years, or at least about 2 years) prior to the first administration of an endothelin receptor antagonist, or a pharmaceutically acceptable salt thereof, and the APRIL binding antibody or antigen-binding fragment thereof. For example, the subject can be excreting from about 0.3 grams to 0.5 grams, 0.5 grams to 1 gram, from about 0.5 grams to 1.5 grams, from about 1 gram to 1.5 grams, or from about 1.5 grams to 2 grams of protein in the urine per day for at least 3 months.

In some embodiments, the subject is excreting at least about 1 gram of protein in the urine per day on at least two of three consecutive measurements a year prior to the first administration of an endothelin receptor antagonist, or a pharmaceutically acceptable salt thereof, and the APRIL binding antibody or antigen-binding fragment thereof. For example, the subject can be excreting about 1.1 grams, 1.2 grams, 1.3 grams, 1.4 grams, 1.5 grams, 1.6 grams, 1.7 grams, 1.8 grams, 1.9 grams, 2.0 grams, 2.1 grams, 2.2 grams, 2.3 grams, 2.4 grams, 2.5 grams, 2.6 grams, 2.7 grams. 2.8 grams, 2.9 grams, 3.0 grams, 3.1 grams, 3.2 grams, 3.3 grams, 3.4 grams, 3.5 grams, 5 grams, or 7.5 grams, or 10 grams of protein, or any value in between, in the urine per day on at least two of three consecutive measurements a vear prior to the first administration of an endothelin receptor antagonist, or a pharmaceutically acceptable salt thereof, and the APRIL binding antibody or antigen-binding fragment thereof.

In some embodiments, the subject has an UACR value of at least about 300 mg/g for at least three months prior to the first administration of an endothelin receptor antagonist, or a pharmaceutically acceptable salt thereof, and the APRIL binding antibody or antigen-binding fragment thereof, for example, 300 mg/g to about 5,000 mg/g. In some embodiments, the subject has an UACR value of about 800 mg/g for at least three months prior to the first administration of an endothelin receptor antagonist, or a pharmaceutically acceptable salt thereof, and the APRIL binding antibody or antigen-binding fragment thereof, for example, 800 mg/g to about 5,000 mg/g. In some embodiments, the subject has an UACR value of at least about 500 mg/g, about 600 mg/g, about 700 mg/g, about 800 mg/g, about 900 mg/g, about 1,000 mg/g, about 1,500 mg/g, about 2,000 mg/g, about 2,500 mg/g, about 3,000 mg/g, about 3,500 mg/g, about 4,000 mg/g, about 4,500 mg/g, or about 5,000 mg/g, or any value in between, for at least three months prior to the first administration of an endothelin receptor antagonist, or a pharmaceutically acceptable salt thereof, and the APRIL binding antibody or antigen-binding fragment thereof.

In some embodiments, the subject has a decrease in UACR value of at least about 30% relative to the subject's average UACR value for at least three months prior to the first administration of an endothelin receptor antagonist, or a pharmaceutically acceptable salt thereof, and the APRIL binding antibody or antigen-binding fragment thereof, for example, a decrease of about 30% to about 100%, relative to the subject's average UACR value for at least three months prior to the first administration of an endothelin receptor antagonist, or a pharmaceutically acceptable salt thereof, and the APRIL binding antibody or antigen-binding fragment thereof. In some embodiments, the subject has a decrease in UACR value of at least about 30%, about 40%, about 50%, about 60%, about 70%, about 80%, about 90%, or about 100%, or any value in between, relative to the subject's average UACR value for at least three months prior to the first administration of an endothelin receptor antagonist, or a pharmaceutically acceptable salt thereof, and the APRIL binding antibody or antigen-binding fragment thereof. In some embodiments, the subject having a decrease in UACR value does not also experience significant sodium retention and/or significant fluid retention. In some embodiments, significant fluid retention can be about 1 kg to about 4 kg over six weeks, for example, about 4 kg, about 3.5 kg, about 3 kg, about 2.5 kg, about 2 kg, about 1.5 kg, or about 1 kg, or any value in between over 6 weeks. In some embodiments, a subject having significant fluid retention exhibits clinical symptoms of edema.

In certain embodiments, the subject has an average eGFR of about 20 to about 90 mL/min/1.73 m² for at least about 3 months prior to the first administration of an endothelin receptor antagonist, or a pharmaceutically acceptable salt thereof, and the APRIL binding antibody or antigen-binding fragment thereof (e.g., about 3 months, about 4 months, about 5 months, about 6 months, about 7 months, about 8 months, about 9 months, about 10 months, about 11 months, about 12 months, about 1.5 years, or about 2 years). For example, about 20 to about 50 mL/min/1.73 m²; about 30 to about 60 mL/min/1.73 m²; about 40 to about 70 mL/min/1.73 m²; about 50 to about 80 mL/min/1.73 m²; or about 60 to about 90 mL/min/1.73 m²; for at least about 3 months prior to the first administration of an endothelin receptor antagonist, or a pharmaceutically acceptable salt thereof, and the APRIL binding antibody or antigen-binding fragment thereof. In some embodiments, the subject has an average eGFR 60 mL/min/1.73 m² for at least about 3 months prior to the first administration of an endothelin receptor antagonist, or a pharmaceutically acceptable salt thereof, and the APRIL binding antibody or antigen-binding fragment thereof. In certain embodiments, the subject has an average eGFR <55 mL/min/1.73 m² for at least about 3 months. In certain embodiments, the subject has an average eGFR <50 mL/min/1.73 m² for at least about 3 months. In certain embodiments, the subject has an average eGFR <45 mL/min/1.73 m² for at least about 3 months. In certain embodiments, the subject has an average eGFR <40 mL/min/1.73 m² for at least about 3 months. In certain embodiments, the subject has an average eGFR 5 35 mL/min/1.73 m² for at least about 3 months. In certain embodiments, the subject has an average eGFR 25 mL/min/I 0.73 m² for at least about 3 months. In certain embodiments, the subject has an average eGFR 5 20 mL/min/1.73 m² for at least about 3 months. In certain of the foregoing embodiments, the subject has an average eGFR between about 30 mL/min/1.73 m² and about 60 mL/min/1.73 m² for at least 3 months before the administration of an endothelin receptor antagonist, or a pharmaceutically acceptable salt thereof, and the APRIL binding antibody or antigen-binding fragment thereof. For example, the subject can have an average eGFR of between about 30 mL/min/1.73 m² and about 55 mL/min/1.73 m², between about 30 mL/min/1.73 m² and about 50 mL/min/1.73 m², between about 30 mL/min/1.73 m² and about 45 mL/min/1.73 m², or between about 30 mL/min/1.73 m² and about 40 mL/min/1.73 m².

In certain embodiments, the subject has an average eGFR of about 30 mL/min/1.73 m² to about 45 mL/min/1.73 m², for example, about ≤45, about ≤40, about ≤35 or about ≤30, for at least about 3 months (e.g., at least about 4 months, at least about 5 months, at least about 6 months, at least about 7 months, at least about 8 months, at least about 9 months, at least about 10 months, at least about 11 months, at least about a year, at least about 1.5 years, or at least about 2 years) prior to the first administration of an endothelin receptor antagonist, or a pharmaceutically acceptable salt thereof, and the APRIL binding antibody or antigen-binding fragment thereof. In some embodiments, the subject has an average eGFR of about 25 mL/min/1.73 m² to about 75 mL/min/1.73 m² for at least about 3 months prior to the first administration of an endothelin receptor antagonist, or a pharmaceutically acceptable salt thereof, and the APRIL binding antibody or antigen-binding fragment thereof. For example, about 25 mL/min/1.73 m², about 30 mL/min/1.73 m², about 35 mL/min/1.73 m², about 40 mL/min/1.73 m², about 45 mL/min/1.73 m², about 50 mL/min/1.73 m², about 55 mL/min/1.73 m², about 60 mL/min/1.73 m², about 65 mL/min/1.73 m², about 70 mL/min/1.73 m², about 75 mL/min/1.73 m², or any value in between, for at least about 3 months prior to the first administration of an endothelin receptor antagonist, or a pharmaceutically acceptable salt thereof, and the APRIL binding antibody or antigen-binding fragment thereof.

In some embodiments, the subject has an average HbA1c of about 4% to about 6% for at least about 3 months (e.g., at least about 4 months, at least about 5 months, at least about 6 months, at least about 7 months, at least about 8 months, at least about 9 months, at least about 10 months, at least about 11 months, at least about a year, at least about 1.5 years, or at least about 2 years) prior to the first administration of an endothelin receptor antagonist, or a pharmaceutically acceptable salt thereof, and the APRIL binding antibody or antigen-binding fragment thereof. For example, the subject can have an average HbA1c of about 4.2%, about 4.4%, about 4.6%, about 4.8%, about 5.0%, about 5.2%, about 5.4%, about 5.6%, about 5.8%, or about 6%, or any value in between.

In some embodiments, the subject has an average fasting blood glucose level of about 125 mg/dL or less for at least about 3 months (e.g., at least about 4 months, at least about 5 months, at least about 6 months, at least about 7 months, at least about 8 months, at least about 9 months, at least about 10 months, at least about 11 months, at least about a year, at least about 1.5 years, or at least about 2 years) prior to the first administration of an endothelin receptor antagonist, or a pharmaceutically acceptable salt thereof, and the APRIL binding antibody or antigen-binding fragment thereof. For example, the subject can have an average fasting blood glucose level of about 120 mg/dL, about 115 mg/dL, about 110 mg/dL, about 105 mg/dL, about 100 mg/dL, about 95 mg/dL, about 90 mg/dL, about 85 mg/dL, about 80 mg/dL, or about 75 mg/dL, or any value in between.

In some embodiments, the subject maintains a potassium level within the normal physiologic range. In certain embodiments, the subject maintains a potassium level within the normal physiologic range for at least about 3 months (e.g., at least about 4 months, at least about 5 months, at least about 6 months, at least about 7 months, at least about 8 months, at least about 9 months, at least about 10 months, at least about 11 months, at least about a year, at least about 1.5 years, or at least about 2 years) prior to the first administration of an endothelin receptor antagonist, or a pharmaceutically acceptable salt thereof, and the APRIL binding antibody or antigen-binding fragment thereof. In certain embodiments, the subject maintains a potassium level within 3.5 to 5.2 mEq/L. For example, the subject maintains an average potassium level at about 3.5 mEq/L, about 3.6 mEq/L, about 3.7 mEq/L, about 3.8 mEq/L, about 3.9, about mEq/L, about 4.0 mEq/L, about 4.1 mEq/L, about 4.2 mEq/L, about 4.3 mEq/L, about 4.4 mEq/L, about 4.5 mEq/L, about 4.6 mEq/L, about 4.7 mEq/L, about 4.8 mEq/L, about 4.9 mEq/L, about 5.0 mEq/L, about 5.1 mEq/L, or about 5.2 mEq/L, or any value in between.

In some embodiments, the subject maintains a sodium level within the normal physiologic range. In certain embodiments, the subject maintains a potassium level within the normal physiologic range for at least about 3 months (e.g., at least about 4 months, at least about 5 months, at least about 6 months, at least about 7 months, at least about 8 months, at least about 9 months, at least about 10 months, at least about 11 months, at least about ayear, at least about 1.5 years, or at least about 2 years) prior to the first administration of an endothelin receptor antagonist, or a pharmaceutically acceptable salt thereof, and the APRIL binding antibody or antigen-binding fragment thereof. In certain embodiments, the subject maintains a sodium level within 135 to 145 mEq/L. For example, the subject maintains an average sodium level of about 135 mEq/L, about 136 mEq/L, about 137 mEq/L, about 138 mEq/L, about 139 mEq/L, about 140 mEq/L, about 141 mEq/L, about 142 mEq/L, about 143 mEq/L, about 144 mEq/L, about or 145 mEq/L, or any value in between.

In some embodiments, the subject has ALT/AST levels during the administration of an endothelin receptor antagonist, or a pharmaceutically acceptable salt thereof, and the APRIL binding antibody or antigen-binding fragment thereof, that are about the same as the ALT/AST levels prior to the first administration of an endothelin receptor antagonist, or a pharmaceutically acceptable salt thereof, and the APRIL binding antibody or antigen-binding fragment thereof. For example, the subject has ALT/AST levels during the administration of an endothelin receptor antagonist, or a pharmaceutically acceptable salt thereof, and the APRIL binding antibody or antigen-binding fragment thereof within about 25%, about 20%, about 15%, about 10%, about 5%, about or 2.5%, or any value in between, of the levels prior to the first administration of an endothelin receptor antagonist, or a pharmaceutically acceptable salt thereof, and the APRIL binding antibody or antigen-binding fragment thereof.

In some embodiments, the subject has bilirubin levels during the administration of an endothelin receptor antagonist, or a pharmaceutically acceptable salt thereof, and the APRIL binding antibody or antigen-binding fragment thereof, that are about the same as the bilirubin levels prior to the first administration of an endothelin receptor antagonist, or a pharmaceutically acceptable salt thereof, and the APRIL binding antibody or antigen-binding fragment thereof. For example, the subject has bilirubin levels during the administration of an endothelin receptor antagonist, or a pharmaceutically acceptable salt thereof, and the APRIL binding antibody or antigen-binding fragment thereof within about 25%, about 20%, about 15%, about 10%, about 5%, or about 2.5%, or any value in between, of the levels prior to the first administration of an endothelin receptor antagonist, or a pharmaceutically acceptable salt thereof, and the APRIL binding antibody or antigen-binding fragment thereof.

In some embodiments, the fluid retention in the subject is manageable with diuretics (e.g., during the treatment with an endothelin receptor antagonist, or a pharmaceutically acceptable salt thereof, and the APRIL binding antibody or antigen-binding fragment thereof and/or prior to the first administration of an endothelin receptor antagonist, or a pharmaceutically acceptable salt thereof, and the APRIL binding antibody or antigen-binding fragment thereof). For example, the fluid retention can be less than about 3 kilograms (kg) of weight gain over 6 weeks. In some embodiments, the fluid retention is less than about 4 kg, about 3.5 kg, about 3 kg, about 2.5 kg, about 2 kg, about 1.5 kg, or about 1 kg, or any value in between over 6 weeks.

In some embodiments, the subject undergoes surgery, and/or other regimens prior to, substantially at the same time as, or following the administration of an endothelin receptor antagonist, or a pharmaceutically acceptable salt thereof, and the APRIL binding antibody or antigen-binding fragment thereof, as described herein. In some embodiments, the subject is administered other chemical and/or biological therapeutic agents prior to, substantially at the same time as, or following the administration of an endothelin receptor antagonist, or a pharmaceutically acceptable salt thereof, and the APRIL binding antibody or antigen-binding fragment thereof, as described herein.

In some embodiments, the subject has been receiving one or more inhibitors of the renin-angiotensin system for at least about 60 weeks prior to the first administration of an endothelin receptor antagonist, or a pharmaceutically acceptable salt thereof, and the APRIL binding antibody or antigen-binding fragment thereof. For example, in some embodiments, the subject has been receiving one or more inhibitors of the renin-angiotensin system for at least about 12 weeks, about 24 weeks, about 48 weeks, or about 60 weeks, or any value in between, prior to the first administration of an endothelin receptor antagonist, or a pharmaceutically acceptable salt thereof, and the APRIL binding antibody or antigen-binding fragment thereof.

In some embodiments, the subject has been receiving a maximally tolerated stable dose of the one or more renin-angiotensin system inhibitors. For example, the subject can be receiving a maximally tolerated stable dose of the one or more renin-angiotensin system inhibitor for at least about 12 weeks, about 14 weeks, about 16 weeks, about 18 weeks, about 20 weeks, about 25 weeks, about 30 weeks, about 35 weeks, about 40 weeks, about 45 weeks, or about 50 weeks, or any value in between, prior to the first administration of an endothelin receptor antagonist, or a pharmaceutically acceptable salt thereof, and the APRIL binding antibody or antigen-binding fragment thereof. In some embodiments, the one or more inhibitors of the renin-angiotensin system is selected from the group consisting of angiotensin converting enzyme (ACE) inhibitors, angiotensin II receptor blockers (ARBs), renin inhibitors, and aldosterone antagonists. For example, the one or more inhibitors of the renin-angiotensin system can be ACE inhibitor, ARB, or a combination thereof, wherein the ACE inhibitor or ARB can be described anywhere herein. For example, the ACE inhibitor can be selected from the group consisting of quinapril, fosinopril perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril. For example, the ARB can be selected from the group consisting of candesartan, candesartan cilexetil, eprosartan, irbesartan, losartan, olmesartan, olmesartan medoxomil, telmisartan, valsartan, azilsartan medoxomil, and BRA-657.

In some embodiments, the subject is also being administered one or more additional agents. In some embodiments, the one or more additional agents are selected from the group consisting of calcineurin inhibitors, proteasome inhibitors, aminoquinolines, complement inhibitors, B-cell inhibitors, cytotoxic agents, mTOR inhibitors, and steroids. In some embodiments, the dosage of the one or more additional agents is decreased after between about 15 days to about 30 days of treatment with an endothelin receptor antagonist, or a pharmaceutically acceptable salt thereof, and the APRIL binding antibody or antigen-binding fragment thereof. In some embodiments, the one or more additional agents are immunosuppressants.

In some embodiments, the subject is not currently receiving one or more additional agents. In certain embodiments, the subject has not used one or more additional agent for two or more weeks within the 6 months prior to the first administration of an endothelin receptor antagonist, or a pharmaceutically acceptable salt thereof, and the APRIL binding antibody or antigen-binding fragment thereof.

In some embodiments, the one or more additional agents are selected from the group consisting of calcineurin inhibitors, proteasome inhibitors, aminoquinolines, complement inhibitors, B-cell inhibitors, cytotoxic agents, mTOR inhibitors, and steroids.

In certain embodiments, the one or more additional agents are steroids. For example, the one or more additional agents can be selected from the group consisting of prednisone, dexamethasone, hydrocortisone, ciclosporin, and combinations of any of the foregoing.

In certain embodiments, the one or more additional agents are aminoquinolines. For example, the one or more additional agents can be hydroxychloroquine.

In some embodiments, the subject is receiving one or more additional agents at the time of treatment with atrasentan. In certain embodiments, the dosage of the one or more additional agents is decreased after treatment with an endothelin receptor antagonist, or a pharmaceutically acceptable salt thereof, and the APRIL binding antibody or antigen-binding fragment thereof (e.g., after 1 week, 2 weeks, 3 weeks. 4 weeks, 5 weeks, 6 weeks, 7 weeks, 8 weeks, 9 weeks, 10 weeks, 20 weeks, 30 weeks, 40 weeks. 50 weeks, 60 weeks. 70 weeks, 80 weeks, 90 weeks, 100 weeks, 110 weeks, 120 weeks, 130 weeks, 140 weeks, 150 weeks, 160 weeks, 170 weeks, 180 weeks, 190 weeks, or 200 weeks of treatment). In certain of these embodiments, the dosage of the one or more additional agents is decreased after between about 15 days to about 30 days of treatment with an endothelin receptor antagonist, or a pharmaceutically acceptable salt thereof, and the APRIL binding antibody or antigen-binding fragment thereof. In certain of the foregoing embodiments, the additional agent dosage is decreased by about 10% to about 100%. In certain embodiments, the additional agent dosage is decreased by about 15% to about 100%. In certain embodiments, the additional agent dosage is decreased by about 20% to about 100%. In certain embodiments, the additional agent dosage is decreased by about 25% to about 100%. In certain embodiments, the additional agent dosage is decreased by about 30% to about 100%. In certain embodiments, the additional agent dosage is decreased by about 35% to about 100%. In certain embodiments, the additional agent dosage is decreased by about 40% to about 100%. In certain embodiments, the additional agent dosage is decreased by about 45% to about 100%. In certain embodiments, the additional agent dosage is decreased by about 50% to about 100%. In certain embodiments, the additional agent dosage is decreased by about 55% to about 100%. In certain embodiments, the additional agent dosage is decreased by about 60% to about 100%. In certain embodiments, the additional agent dosage is decreased by about 65% to about 100%. In certain embodiments, the additional agent dosage is decreased by about 70% to about 100%. In certain embodiments, the additional agent dosage is decreased by about 75% to about 100%. In certain embodiments, the additional agent dosage is decreased by about 80% to about 100%. In certain embodiments, the additional agent dosage is decreased by about 85% to about 100%. In certain embodiments, the additional agent dosage is decreased by about 90% to about 100%. In certain of the foregoing embodiments, the dosage of the one or more additional agents is decreased after between about 15 days to about 30 days (e.g., about 15 days, about 20 days, about 25 days, or about 30 days) of treatment with an endothelin receptor antagonist, or a pharmaceutically acceptable salt thereof, and the APRIL binding antibody or antigen-binding fragment thereof. When the dosage of additional agent is decreased by 100% as described herein, the subject is no longer needing additional agent.

In certain embodiments, the dosage of one or more steroids is decreased after treatment with an endothelin receptor antagonist, or a pharmaceutically acceptable salt thereof, and the APRIL binding antibody or antigen-binding fragment thereof, for example, after between about 15 days to about 30 days of treatment with an endothelin receptor antagonist, or a pharmaceutically acceptable salt thereof, and the APRIL binding antibody or antigen-binding fragment thereof. In some embodiments, the steroid dosage is decreased by about 10% to about 100%, as described herein. In some embodiments, the dosage of prednisone, dexamethasone, hydrocortisone, ciclosporin, or a combination of any of the foregoing is reduced by about 10% to about 100% after treatment with an endothelin receptor antagonist, or a pharmaceutically acceptable salt thereof, and the APRIL binding antibody or antigen-binding fragment thereof.

In certain embodiments, the dosage of one or more aminoquinolines is decreased after treatment with an endothelin receptor antagonist, or a pharmaceutically acceptable salt thereof, and the APRIL binding antibody or antigen-binding fragment thereof, for example, after between about 15 days to about 30 days of treatment with an endothelin receptor antagonist, or a pharmaceutically acceptable salt thereof, and the APRIL binding antibody or antigen-binding fragment thereof. In some embodiments, the aminoquinoline dosage is decreased by about 10% to about 100%, as described herein. In some embodiments, the dosage of hydroxychloroquine is reduced by about 10% to about 100% after treatment with an endothelin receptor antagonist, or a pharmaceutically acceptable salt thereof, and the APRIL binding antibody or antigen-binding fragment thereof.

In some embodiments, the subject is concomitantly receiving one or more additional therapeutic agents. The one or more additional therapeutic agents are described herein. For example, the subject is concomitantly receiving an inhibitor of one or more elements of the renin-angiotensin-aldosterone system. In certain embodiments, the subject is concomitantly receiving a SGLT-2 inhibitor, an ACE inhibitor, an ARB, a statin, a diuretic, a calcium channel blocker, a beta blocker, an aldosterone antagonist, fish oil, hydroxychloroquine, or a combination of any of the foregoing. In certain of these embodiments, the subject is concomitantly receiving a SGLT-2 inhibitor. In certain of these embodiments, the subject is concomitantly receiving an ACE inhibitor, an ARB, or a combination thereof. In certain embodiments, the subject is concomitantly receiving one or more statins, such as atorvastatin, fluvastatin, lovastatin, pravastatin, rosuvastatin, simvastatin, and pitavastatin. In certain embodiments, the subject is concomitantly receiving one or more diuretics, such as hydrochlorothiazide, trichlormethiazide, hydroflumethiazide, quinethazone, metolazone, chlorothiazide, chlorthalidone, indapamide, methyclothiazide bemetanide, torsemide, piretanide, ethacrynic acid, bumetanide, furosemide, triamterene, spironolactone, eplerenone, and amiloride. In certain embodiments, the subject is concomitantly receiving a SGLT-2 inhibitor, such as canagliflozin, dapagliflozin, empagliflozin, or ertugliflozin. In certain embodiments, the subject is concomitantly receiving one or more ACE inhibitors, such as quinapril, fosinopril perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril. In certain embodiments, the subject is concomitantly receiving an ARB, such as candesartan, candesartan cilexetil, eprosartan, irbesartan, losartan, olmesartan, olmesartan medoxomil, telmisartan, valsartan, azilsartan medoxomil, and BRA-657. In certain embodiments, the subject is concomitantly receiving a diuretic and an ACE inhibitor or an ARB. In certain embodiments, the subject is concomitantly receiving a diuretic, an ACE inhibitor, and an ARB. In certain embodiments, the subject is concomitantly receiving a diuretic and a SGLT-2 inhibitor, and an ACE inhibitor or an ARB. In certain embodiments, the subject is concomitantly receiving a diuretic, a SGLT-2 inhibitor, an ACE inhibitor, and an ARB. In certain embodiments, the subject concomitantly receiving one or more additional therapeutic agents has not previously received the one or more therapeutic agents. For example, a subject that is concomitantly receiving a SGLT-2 inhibitor that has not previously received a SGLT-2 inhibitor.

In some embodiments, the subject has previously received, but is not concomitantly receiving, one or more additional therapeutic agents such as those described herein. For example, the subject is has previously received, but is not concomitantly receiving a SGLT-2 inhibitor, an ACE inhibitor, an ARB, a statin, a diuretic, a calcium channel blocker, a beta blocker, an aldosterone antagonist, fish oil, hydroxychloroquine, or a combination of any of the foregoing, as described herein. In certain of these embodiments, the subject has previously received, but is not concomitantly receiving a SGLT-2 inhibitor.

In some embodiments, the subject has cellular glomerular crescents present in about ≤25% of glomeruli within 6 months prior to the first administration of an endothelin receptor antagonist, or a pharmaceutically acceptable salt thereof, and the APRIL binding antibody or antigen-binding fragment thereof. For example, the subject can have cellular glomerular crescents present in about 25%, about 20%, about 15%, about 10%, about 5%, or about 1%, or any value in between, of glomeruli. In some embodiments, the subject does not have cellular glomerular crescents present in the glomeruli. In certain embodiments, the subject is not under clinical suspicion of rapidly progressive glomerulonephritis (RPGN).

In some embodiments, the subject has not undergone organ transplantation prior to the first administration of an endothelin receptor antagonist, or a pharmaceutically acceptable salt thereof, and the APRIL binding antibody or antigen-binding fragment thereof.

In some embodiments, the subject has a systolic blood pressure of below about 160 mmHg prior to the first administration of an endothelin receptor antagonist, or a pharmaceutically acceptable salt thereof, and the APRIL binding antibody or antigen-binding fragment thereof. For example, the subject can be a systolic blood pressure of below about 155 mmHg, below about 150 mmHg, below about 145 mmHg. or below about 140 mmHg. In some embodiments, the subject has a diastolic blood pressure of below about 100 mmHg prior to the first administration of an endothelin receptor antagonist, or a pharmaceutically acceptable salt thereof, and the APRIL binding antibody or antigen-binding fragment thereof. For example, the subject can have a diastolic blood pressure of below about 100 mmHg, below about 95 mmHg, or below about 90 mmHg. In some embodiments, the subject has a systolic blood pressure of between about 100 mm Hg and about 130 mm Hg and a diastolic blood pressure of about 70 mm Hg to about 90 mm Hg.

In some embodiments, the subject has not been diagnosed with heart failure prior to the first administration of an endothelin receptor antagonist, or a pharmaceutically acceptable salt thereof, and the APRIL binding antibody or antigen-binding fragment thereof. In some embodiments, the subject has not been previously admitted to hospital for conditions relating to fluid overload prior to the first administration of an endothelin receptor antagonist, or a pharmaceutically acceptable salt thereof, and the APRIL binding antibody or antigen-binding fragment thereof. Non-limiting examples of conditions include uncontrolled peripheral edema, pleural effusion, or ascites. In some embodiments, the subject has not been diagnosed with clinically significant liver disease prior to the first administration of an endothelin receptor antagonist, or a pharmaceutically acceptable salt thereof, and the APRIL binding antibody or antigen-binding fragment thereof. In some embodiments, the transaminase or bilirubin values of the subject are no more than twice the normal upper limit prior to the first administration of an endothelin receptor antagonist, or a pharmaceutically acceptable salt thereof, and the APRIL binding antibody or antigen-binding fragment thereof. For example, the ALT level of the subject is below about 110 U/L (e.g., below about 100 U/L, below 90 U/L, below about 80 U/L, below about 70 U/L, below about 60 U/L, below about 50 U/L, or below about 40 U/L, or any value in between). As another example, the AST level of the subject is below 100 U/L (e.g., below 90 U/L, below about 80 U/L, below about 70 U/L, below about 60 U/L, below about 50 U/L, or below about 40 U/L, or any value in between). As yet another example, the bilirubin level of the subject is below about 2.5 mg/dL (e.g., below about 2 mg/dL, below about 1.5 mg/dL, below about 1.4 mg/dL, below about 1.3 mg/dL, below about 1.2 mg/dL, below about 1.1 mg/dL, below about 1.0 mg/dL, or below about 0.9 mg/dL, or any value in between).

In some embodiments, the subject has a hemoglobin level of above about 9 g/dL (e.g., above about 10 g/dL, about 11 g/dL, about 12 g/dL, or about 13 g/dL, or any value in between) prior to the first administration of an endothelin receptor antagonist, or a pharmaceutically acceptable salt thereof, and the APRIL binding antibody or antigen-binding fragment thereof. In some embodiments, the subject has not received blood transfusion for anemia for at least about 3 months (e.g., at least about 4 months, about 5 months, about 6 months, or about one year) prior to the first administration of an endothelin receptor antagonist, or a pharmaceutically acceptable salt thereof, and the APRIL binding antibody or antigen-binding fragment thereof. In some embodiments, the subject has not been diagnosed with cancer for at least 5 years prior to the first administration of an endothelin receptor antagonist, or a pharmaceutically acceptable salt thereof, and the APRIL binding antibody or antigen-binding fragment thereof. In some embodiments, the subject has not been diagnosed with cancer (e.g., lung cancer or prostate cancer) for at least 5 years prior to the first administration of an endothelin receptor antagonist, or a pharmaceutically acceptable salt thereof, and the APRIL binding antibody or antigen-binding fragment thereof. In some embodiments, the subject has not been diagnosed with cancer for at least 5 years prior to the first administration of an endothelin receptor antagonist, or a pharmaceutically acceptable salt thereof, and the APRIL binding antibody or antigen-binding fragment thereof, unless the cancer is nonmelanoma skin cancer not requiring ongoing treatment. In some embodiments, the subject does not have cancer prior to the first administration of an endothelin receptor antagonist, or a pharmaceutically acceptable salt thereof, and the APRIL binding antibody or antigen-binding fragment thereof, unless the cancer is nonmelanoma skin cancer not requiring ongoing treatment. In some embodiments, the subject does not suffer from cancer prior to the first administration of an endothelin receptor antagonist, or a pharmaceutically acceptable salt thereof, and the APRIL binding antibody or antigen-binding fragment thereof, unless the cancer is nonmelanoma skin cancer not requiring ongoing treatment. In some embodiments, the subject is not being treated for cancer for at least 5 years prior to the first administration of an endothelin receptor antagonist, or a pharmaceutically acceptable salt thereof, and the APRIL binding antibody or antigen-binding fragment thereof, unless the cancer is nonmelanoma skin cancer not requiring ongoing treatment.

In some embodiments, the subject has been determined to have controlled serum glucose levels. In certain embodiments, the subject has been determined to have controlled serum glucose levels. For example, the subject has been determined to have a fasting serum glucose level of below about 130 mg/dL, about 125 mg/dL, about 120 mg/dL, about 115 mg/dL, about 110 mg/dL, about 105 mg/dL, about 100 mg/dL, about 95 mg/dL, about 90 mg/dL, about 85 mg/dL, about 80 mg/dL, or about 75 mg/dL, or any value in between.

In some embodiments, the subject has not been previously diagnosed with a chronic kidney disease that is other than IgA nephropathy. Non-limiting examples include a diabetic kidney disease, a hypertensive kidney disease, or a primary glomerulopathy that is determined to not be associated with IgA nephropathy. In certain embodiments, the subject has not been previously diagnosed with a diabetic kidney disease. In certain embodiments, the subject has not been previously diagnosed with a hypertensive kidney disease. In certain embodiments, the subject has not been diagnosed with a primary glomerulopathy that is determined to not be associated with IgA nephropathy.

In some embodiments, the subject does not have a chronic kidney disease that is other than IgA nephropathy. Non-limiting examples include a diabetic kidney disease, a hypertensive kidney disease, or a primary glomerulopathy that is determined to not be associated with IgA nephropathy. In certain embodiments, the subject does not have a diabetic kidney disease. In certain embodiments, the subject does not have a hypertensive kidney disease. In certain embodiments, the subject does not have a primary glomerulopathy that is determined to not be associated with IgA nephropathy.

In some embodiments, the subject does not suffer from a chronic kidney disease that is other than IgA nephropathy. Non-limiting examples include a diabetic kidney disease, a hypertensive kidney disease, or a primary glomerulopathy that is determined to not be associated with IgA nephropathy. In certain embodiments, the subject does not suffer from a diabetic kidney disease. In certain embodiments, the subject does not suffer from a hypertensive kidney disease. In certain embodiments, the subject does not suffer from a primary glomerulopathy that is determined to not be associated with IgA nephropathy.

In some embodiments, the subject is not being treated for a chronic kidney disease that is other than IgA nephropathy. Non-limiting examples include a diabetic kidney disease, a hypertensive kidney disease, or a primary glomerulopathy that is determined to not be associated with IgA nephropathy. In certain embodiments, the subject is not being treated for a diabetic kidney disease. In certain embodiments, the subject is not being treated for a hypertensive kidney disease. In certain embodiments, the subject is not being treated for a primary glomerulopathy that is determined to not be associated with IgA nephropathy.

### Treatment Outcome

In some embodiments of the methods, uses, or product for uses herein, renal inflammation is decreased after treatment with an endothelin receptor antagonist, or a pharmaceutically acceptable salt thereof, and the APRIL binding antibody or antigen-binding fragment thereof. In some embodiments, renal inflammation in the subject is decreased by at least about 10% after treatment with an endothelin receptor antagonist, or a pharmaceutically acceptable salt thereof, and the APRIL binding antibody or antigen-binding fragment thereof (e.g., after treatment for 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In some embodiments, the renal inflammation in the subject is decreased by at least about 20%, about 30%, about 40%, about 50%, about 60% about 70% about 80%, about 90%, or about 95%, or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In some embodiments, renal fibrosis is decreased after treatment with an endothelin receptor antagonist, or a pharmaceutically acceptable salt thereof, and the APRIL binding antibody or antigen-binding fragment thereof. In some embodiments, renal fibrosis in the subject is decreased by at least about 10% after treatment with an endothelin receptor antagonist, or a pharmaceutically acceptable salt thereof, and the APRIL binding antibody or antigen-binding fragment thereof (e.g., after treatment for 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, the renal fibrosis in the subject is decreased by at least about 20%, about 30%, about 40%, about 50%, about 60% about 70% about 80%, about 90%, or about 95%, or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In some embodiments, renal fibrosis in the subject is decreased to less than about 50% of the cortical area after treatment with an endothelin receptor antagonist, or a pharmaceutically acceptable salt thereof, and the APRIL binding antibody or antigen-binding fragment thereof (e.g., after treatment for 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, renal fibrosis in the subject is decreased to less than about 40% of the cortical area. For example, in some embodiments, renal fibrosis in the subject is decreased to less than about 35%, about 30%, about 25%, about 20%, about 15%, or about 10%, or any value in between, of the cortical area. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In some embodiments, the occurrence of hematuria is decreased in a subject after treatment with an endothelin receptor antagonist, or a pharmaceutically acceptable salt thereof, and the APRIL binding antibody or antigen-binding fragment thereof. In some embodiments, the number of urinary red blood cells per high powered (microscope) field (rbc/hpf) in the subject is decreased by at least about 10% after treatment with an endothelin receptor antagonist, or a pharmaceutically acceptable salt thereof, and the APRIL binding antibody or antigen-binding fragment thereof (e.g., after treatment for 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks). In certain embodiments, the urinary rbc/hpf in the subject is decreased by at least about 20%. For example, in some embodiments, the urinary rbc/hpf in the subject is decreased by at least about 30%, about 40%, about 50%, about 60% about 70% about 80%, about 90%, or about 95%, or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In some embodiments, the rate of decrease of eGFR of the subject is reduced by at least about 10% after treatment with an endothelin receptor antagonist, or a pharmaceutically acceptable salt thereof, and the APRIL binding antibody or antigen-binding fragment thereof (e.g., after treatment for 1 week, 2 weeks, 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, the rate of decrease of eGFR of the subject is reduced by at least about 20%. For example, in some embodiments, the rate of decrease of eGFR of the subject is reduced by at least about 30%, about 40%, about 50%, about 60% about 70% about 80%, about 90%, or about 95%, or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days. In certain of the foregoing embodiments, the subject has been treated with an endothelin receptor antagonist, or a pharmaceutically acceptable salt thereof, and the APRIL binding antibody or antigen-binding fragment thereof for between about 6 months and about 1 year.

In some embodiments, the rate of decrease of eGFR of the subject is reduced to below about 10 mL/min per year after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In some embodiments, the rate of decrease of eGFR of the subject is reduced to below about 9 mL/min per year. For example, in some embodiments, the rate of decrease of eGFR of the subject is reduced to below about 8 mL/min per year, about 7 mL/min per year, about 6 mL/min per year, about 5 mL/min per year, about 4 mL/min per year, about 3 m/min per year, about 2 mL/min per year, or about 1 mL/min per year, or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days. In certain of the foregoing embodiments, the subject has been treated with an endothelin receptor antagonist, or a pharmaceutically acceptable salt thereof, and the APRIL binding antibody or antigen-binding fragment thereof for between about 6 months and about 1 year.

In some embodiments, the risk of the subject developing ESRD is reduced by about 20% to about 99% after treatment with an endothelin receptor antagonist, or a pharmaceutically acceptable salt thereof, and the APRIL binding antibody or antigen-binding fragment thereof (e.g., after treatment for 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). For example, the risk of the subject developing ESRD can be reduced by about 20%, about 25%, about 30%, about 35%, about 40%, about 45%, about 50%, about 55%, about 60%, about 65%, about 70%, about 75%, about 80%, about 85%, about 90%, or about 99%, or any value in between. In certain of the foregoing embodiments, the subject has been treated for between about 90 days to about 180 days. In certain embodiments, the risk of the subject developing ESRD is reduced by about 20% to about 99% after between about 90 and about 180 days of treatment with an endothelin receptor antagonist, or a pharmaceutically acceptable salt thereof, and the APRIL binding antibody or antigen-binding fragment thereof. In certain of the foregoing embodiments, the subject has been treated with an endothelin receptor antagonist, or a pharmaceutically acceptable salt thereof, and the APRIL binding antibody or antigen-binding fragment thereof for between about 6 months and about 1 year.

In some embodiments, the method increases the time between the diagnosis of IgA nephropathy in the subject and the time when eGFR of the subject falls below about 15 mL/min/1.73 m². In certain embodiments, the method increases the time between the diagnosis of IgA nephropathy in the subject and the time when eGFR of the subject falls below 15 mL/min/1.73 m² by at least about 10%. For example, in some embodiments, the method increases the time between the diagnosis of IgA nephropathy in the subject and the time when eGFR of the subject falls below about 15 mL/min/1.73 m² by at least about 20%, about 30%, about 40%, about 50%, about 60%, about 70%, about 80%, about 90%, about 95%, about 100%, about 150%, about 200%, about 250%, about 300%, about 350%, about 400%, about 450%, or about 500%, or any value in between.

In some embodiments, the method increases the time between the diagnosis of IgA nephropathy in the subject and the time when eGFR of the subject falls below 15 mL/min/1.73 m² by at least about 1 year. For example, the method can delay the time when eGFR of the subject falls below 15 mL/min/1.73 m² by at least about 1.5 years, about 2 years, about 2.5 years, about 3 years, about 3.5 years, about 4 years, about 4.5 years, about 5 years, about 5.5 years, about 6 years, about 6.5 years, about 7 years, about 7.5 years, about 8 years, about 8.5 years, about 9 years, about 9.5 years, about 10 years, about 11 years, about 12 years, about 13 years, about 15 years, about 15 years, about 16 years, about 17 years, about 18 years, about 19 years, or about 20 years, or any value in between.

In some embodiments, the method reduces the average rate of decrease in eGFR by from about 0.75 mL/min/year to about 6 mL/min/year for at least about 3 months (e.g., at least about 4 months, at least about 5 months, at least about 6 months, at least about 7 months, at least about 8 months, at least about 9 months, at least about 10 months, at least about 11 months, at least about a year, at least about 1.5 years, or at least about 2 years) prior to the first administration of an endothelin receptor antagonist, or a pharmaceutically acceptable salt thereof, and the APRIL binding antibody or antigen-binding fragment thereof. For example, the method reduces the average rate of decrease in eGFR by about 0.75 mL/min/year, about 1 mL/min/year, about 1.5 mL/min/year, about 2 mL/min/year, about 2.5 mL/min/year, about 3 mL/min/year, about 3.5 mL/min/vear, about 4 m/min/year, about 4.5 mL/min/year, about 5 mL/min/year, about 5.5 L/min/year, or about 6 mL/min/year. In some embodiments, the method reduces the average rate of decrease in eGFR by from about 4 mL/min/year to about 5 mL/min/year for at least about 3 months prior to the first administration of an endothelin receptor antagonist, or a pharmaceutically acceptable salt thereof, and the APRIL binding antibody or antigen-binding fragment thereof. In some embodiments, the method reduces the average rate of decrease in eGFR by from about 3 mL/min/year to about 6 L/min/year for at least about 3 months prior to the first administration of an endothelin receptor antagonist, or a pharmaceutically acceptable salt thereof, and the APRIL binding antibody or antigen-binding fragment thereof. In some embodiments, the method reduces the average rate of decrease in eGFR by from about 4 mL/min/year to about 5 mL/min/year for at least about 3 months prior to the first administration of an endothelin receptor antagonist, or a pharmaceutically acceptable salt thereof, and the APRIL binding antibody or antigen-binding fragment thereof. In some embodiments, the decrease in eGFR in mL/min/year refers to units per 1.73 m².

In some embodiments, the method reduces the average rate of decrease in eGFR by from about 15% to about 30% after between about 6 months and about 24 months of treatment with an endothelin receptor antagonist, or a pharmaceutically acceptable salt thereof, and the APRIL binding antibody or antigen-binding fragment thereof. In some embodiments, the average rate of decrease in eGFR may be reduced by about 15%, after about 6 months, 9, months, 12 months, 15 months, 18 months, 21 months, or 24 months of treatment with an endothelin receptor antagonist, or a pharmaceutically acceptable salt thereof, and the APRIL binding antibody or antigen-binding fragment thereof. In some embodiments, the average rate of decrease in eGFR may be reduced by about 20%, after about 6 months, 9, months, 12 months, 15 months, 18 months, 21 months, or 24 months of treatment with an endothelin receptor antagonist, or a pharmaceutically acceptable salt thereof, and the APRIL binding antibody or antigen-binding fragment thereof. In some embodiments, the average rate of decrease in eGFR may be reduced by about 25%, after about 6 months, 9, months. 12 months, 15 months, 18 months, 21 months, or 24 months of treatment with an endothelin receptor antagonist, or a pharmaceutically acceptable salt thereof, and the APRIL binding antibody or antigen-binding fragment thereof. In some embodiments, the average rate of decrease in eGFR may be reduced by about 30%, after about 6 months, 9, months, 12 months, 15 months, 18 months, 21 months, or 24 months of treatment with an endothelin receptor antagonist, or a pharmaceutically acceptable salt thereof, and the APRIL binding antibody or antigen-binding fragment thereof.

In another aspect, provided herein is a method of decreasing proteinuna, comprising administering an endothelin receptor antagonist, or a pharmaceutically acceptable salt thereof, and an APRIL binding antibody or an antigen-binding fragment thereof, to a subject in need thereof.

In some embodiments, the amount of proteins (e.g., albumin) in the urine of the subject is reduced by at least about 10% after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In some embodiments, the amount of proteins in the urine of the subject is reduced by at least about 15%. For example, in some embodiments, the amount of proteins in the urine of the subject is reduced by at least about 20%, about 30%, about 40%, about 50%, about 60%, about 70%, about 80%, about 90%, or about 95%, or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In certain embodiments, the amount of proteins (e.g., albumin) in the urine of the subject is reduced by about 20% to about 80% after between about 15 day and about 30 days of treatment with an endothelin receptor antagonist, or a pharmaceutically acceptable salt thereof, and the APRIL binding antibody or antigen-binding fragment thereof.

In certain of these embodiments, the amount of proteins in the urine of the subject is reduced by about 25% to about 80%. In certain of these embodiments, the amount of proteins in the urine of the subject is reduced by about 30% to about 80%. In certain of these embodiments, the amount of proteins in the urine of the subject is reduced by about 35% to about 80%. In certain of these embodiments, the amount of proteins in the urine of the subject is reduced by about 40% to about 80%. In certain of these embodiments, the amount of proteins in the urine of the subject is reduced by about 45% to about 80%. In certain of these embodiments, the amount of proteins in the urine of the subject is reduced by about 50% to about 80%.

In some embodiments, the amount of proteins (e.g., albumin) in the urine of the subject is reduced by about 100 mg/dL to about 3,000 mg/dL after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, the amount of proteins in the urine of the subject is reduced by about 100 mg/dL to about 2,500 mg/dL. In certain embodiments, the amount of proteins in the urine of the subject is reduced by about 100 mg/dL to about 2,000 mg/dL. In certain embodiments, the amount of proteins in the urine of the subject is reduced by about 100 mg/dL to about 1,500 mg/dL. In certain embodiments, the amount of proteins in the urine of the subject is reduced by about 100 mg/dL to about 1,000 mg/dL. In certain embodiments, the amount of proteins in the urine of the subject is reduced by about 100 mg/dL to about 500 mg/dL. In certain embodiments, the amount of proteins in the urine of the subject is reduced by about 100 mg/dL to about 400 mg/dL. In certain embodiments, the amount of proteins in the urine of the subject is reduced by about 100 mg/dL to about 300 mg/dL. In certain embodiments, the amount of proteins in the urine of the subject is reduced by about 100 mg/dL to about 200 mg/dL. In certain embodiments, the amount of proteins in the urine of the subject is reduced by about 500 mg/dL to about 2,500 mg/dL. In certain embodiments, the amount of proteins in the urine of the subject is reduced by about 500 mg/dL to about 2,000 mg/dL. In certain embodiments, the amount of proteins in the urine of the subject is reduced by about 500 mg/dL to about 1,500 mg/dL. In certain embodiments, the amount of proteins in the urine of the subject is reduced by about 500 mg/dL to about 1,000 mg/dL. In certain embodiments, the amount of proteins in the urine of the subject is reduced by about 500 mg/dL to about 900 mg/dL. In certain embodiments, the amount of proteins in the urine of the subject is reduced by about 500 mg/dL to about 800 mg/dL. In certain embodiments, the amount of proteins in the urine of the subject is reduced by about 600 mg/dL to about 900 mg/dL. In certain embodiments, the amount of proteins in the urine of the subject is reduced by about 700 mg/dL to about 900 mg/dL. In certain embodiments, the amount of proteins in the urine of the subject is reduced by about 1,000 mg/dL to about 2,000 mg/dL. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In certain embodiments, the amount of proteins (e.g., albumin) in the urine of the subject is reduced by about 100 mg/dL to about 500 mg/dL after between about 15 days and about 30 days of treatment with an endothelin receptor antagonist, or a pharmaceutically acceptable salt thereof, and the APRIL binding antibody or antigen-binding fragment thereof. In certain embodiments, the amount of proteins in the urine of the subject is reduced by about 200 mg/dL to about 500 mg/dL after between about 15 days and about 30 days of treatment with an endothelin receptor antagonist, or a pharmaceutically acceptable salt thereof, and the APRIL binding antibody or antigen-binding fragment thereof. In certain embodiments, the amount of proteins in the urine of the subject is reduced by about 300 mg/dL to about 500 mg/dL after between about 15 days and about 30 days of treatment with an endothelin receptor antagonist, or a pharmaceutically acceptable salt thereof, and the APRIL binding antibody or antigen-binding fragment thereof.

In certain embodiments, the amount of proteins (e.g., albumin) in the urine of the subject is reduced by about 500 mg/dL to about 900 mg/dL after between about 15 days and about 30 days of treatment with an endothelin receptor antagonist, or a pharmaceutically acceptable salt thereof, and the APRIL binding antibody or antigen-binding fragment thereof. In certain embodiments, the amount of proteins in the urine of the subject is reduced by about 600 mg/dL to about 900 mg/dL after between about 15 days and about 30 days of treatment with an endothelin receptor antagonist, or a pharmaceutically acceptable salt thereof, and the APRIL binding antibody or antigen-binding fragment thereof. In certain embodiments, the amount of proteins in the urine of the subject is reduced by about 700 mg/dL to about 900 mg/dL after between about 15 days and about 30 days of treatment with an endothelin receptor antagonist, or a pharmaceutically acceptable salt thereof, and the APRIL binding antibody or antigen-binding fragment thereof.

In some embodiments, the subject has a reduced level of proteins (e.g., albumin) in the urine of below about 1.0 gram/day after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks). In some embodiments, the subject has a reduced level of proteins in the urine of below about 0.9 gram/day. For example, in some embodiments, the subject has a reduced level of proteins in the urine of below about 0.8 gram/day, about 0.7 gram/day, about 0.6 gram/day, 0.5 gram/day, about 0.4 gram/day, about 0.3 gram/day, or about 0.2 gram/day, or any value in between. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days.

In some embodiments, the subject is between about 15 and about 40 years old. In some embodiments, the subject is between about 15 to about 25 years old, about 20 to about 30 years old, about 25 to about 35 years old, about 30 to about 40 years old, or any age in between. In some embodiments, the subject is between about 20 to about 30 years old, or any age in between. In some embodiments, the subject is about 20 years old, about 21 years old, about 22 years old, about 23 years old, about 24 years old, about 25 years old, about 26 years old, about 27 years old, about 28 years old, about 29 years old, or about 30 years old.

In some embodiments, the level of fatigue of the patient is reduced following treatment with an endothelin receptor antagonist, or a pharmaceutically acceptable salt thereof, and the APRIL binding antibody or antigen-binding fragment thereof. In some embodiments, the fatigue is reduced by about 5% to about 80% after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, the fatigue is reduced by about 10% to about 75%. In certain embodiments, the fatigue is reduced by about 10% to about 70%. In certain embodiments, the fatigue is reduced by about 10% to about 65%. In certain embodiments, the fatigue is reduced by about 10% to about 60%. In certain embodiments, the fatigue is reduced by about 10% to about 55%. In certain embodiments, the fatigue is reduced by about 10% to about 50%. In certain embodiments, the fatigue is reduced by about 10% to about 45%. In certain embodiments, the fatigue is reduced by about 10% to about 40%. In certain embodiments, the fatigue is reduced by about 10% to about 35%. In certain embodiments, the fatigue is reduced by about 10% to about 30%. In certain embodiments, the fatigue is reduced by about 10% to about 25%. In certain embodiments, the fatigue is reduced by about 10% to about 20%. In certain embodiments, the fatigue is reduced by about 10% to about 15%. In certain of the foregoing embodiments, the subject has been treated with an endothelin receptor antagonist, or a pharmaceutically acceptable salt thereof, and the APRIL binding antibody or antigen-binding fragment thereof for between about 15 days and about 30 days. In certain embodiments, the decrease in fatigue comprises a decrease in the score on one or more of the Fatigue Severity Scale, the Chalder Fatigue Scale, the FACIT Fatigue Scale, the Brief Fatigue Inventory, the FACT-F Subscale, Global Vigor and Affect, the May and Kline Adjective Checklist, the Pearson-Byars Fatigue Feeling Checklist, the Rhoten Fatigue Scale, the Schedule of Fatigue and Anergia, or the Checklist Individual Strength.

Some embodiments provide a method of inhibiting mesangial cell activation in a subject having IgA nephropathy, comprising administering an endothelin receptor antagonist, or a pharmaceutically acceptable salt thereof, and an APRIL binding antibody or an antigen-binding fragment thereof, to the subject.

Some embodiments provide a method of inhibiting PDGF signaling activity (e.g., decreasing the expression and/or activity of one or more of PIK3R1, PDGFRA, NFKBIA, PIK3CG, PLA2G4A, TIAM1, PDGFB, NFKB1, and MAP3K1) in a mesangial cell in a subject having IgA nephropathy, comprising administering an endothelin receptor antagonist, or a pharmaceutically acceptable salt thereof, and an APRIL binding antibody or an antigen-binding fragment thereof, to the subject.

Some embodiments provide a method of inhibiting mesangial cell activation, comprising contacting a mesangial cell with an endothelin receptor antagonist, or a pharmaceutically acceptable salt thereof, and an APRIL binding antibody or an antigen-binding fragment thereof.

In some embodiments, the mesangial activation is induced by IgA immune complexes. In some embodiments, the mesangial activation is associated with the presence of IgA immune complexes. The presence and/or amount of IgA immune complexes can be detected by a variety of methods. For example, the complexes may be detected in serum or urine, and can also be detected in a kidney biopsy sample.

In some embodiments, the inhibiting of mesangial cell activation comprises reducing expression and/or activity of one or more biomarkers indicative of mesangial cell proliferation. In some embodiments, inhibiting of mesangial cell activation comprises reducing mesangial cell inflammation. In some embodiments, reducing mesangial cell inflammation comprises reducing expression and/or activity of one or more of IL6, MCP1, or other biomarkers indicative of mesangial cell inflammation. In some embodiments, reducing mesangial cell inflammation comprises reducing expression and/or activity of IL-6.

In some embodiments, the expression and/or activity of one or more biomarkers indicative of mesangial cell inflammation is reduced by about 25% to about 99% after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In some embodiments, the expression and/or activity of one or more biomarkers indicative of mesangial cell inflammation is reduced by about 25% to about 50%, about 40% to about 60%, about 50% to about 75%, about 60% to about 80%, about 75% to about 90%, about 85% to about 99%, or any value in between. For example, in some such embodiments, the one or more biomarkers can be IL-6.

In some embodiments, inhibiting of mesangial cell activation comprises reducing mesangial cell inflammation. In some embodiments, reducing mesangial cell inflammation comprises reducing IL-6 signaling (e.g., reducing the expression and/or activity in one or more proteins involved in an IL-6 signaling pathway, e.g., a reduction in the expression and/or activity of one or more of Cntfr, Illb, Csfl, Il2ra, Map3k8, and Illr1). In some embodiments, reducing mesanial cell inflammation comprises reducing the expression and/or activity of one or more (e.g., 1, 2, 3, 4, or 5) of. Cntfr, Illb, Csfl, Il2ra, Map3k8, Illr1.

In some embodiments, the inhibiting of mesangial cell activation comprises reducing the pro-fibrotic response in the mesangial cells. In some embodiments, reducing the pro-fibrotic response in the mesangial cells comprises reducing expression and/or activity of one or more of NF-KB, TGF, PDGF, CTGF, MMP, TIMPS, or other biomarkers indicative of mesangial cell fibrosis. In some embodiments, the expression and/or activity of one or more of NF-KB, TGF, PDGF, CTGF, MMP, and TIMPS, is reduced by about 25% to about 99% after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between), relative to the expression and/or activity prior to administration of an endothelin receptor antagonist, or a pharmaceutically acceptable salt thereof, and the APRIL binding antibody or antigen-binding fragment thereof. In some embodiments, the expression and/or activity of one or more of NF-KB, TGF, PDGF, CTGF, MMP, and TIMPS is reduced by about 25% to about 50%, about 40% to about 60%, about 50% to about 75%, about 60% to about 80%, about 75% to about 90%, about 85% to about 99%, or any value in between.

In some embodiments, inhibiting of mesangial cell activation comprises reducing the pro-fibrotic response in the mesangial cells. In some embodiments, reducing the pro-fibrotic response comprises reducing NF-KB signaling. In some embodiments, reducing the pro-fibrotic response comprises reducing the expression and/or activity of one or more (e.g., 1, 2, 3, 4, or 5) of Pflkb3, Nr4al, Gem. Fosl2, Klf4, F3. Nfkbia, Ifit2, Nr4a2, Klf2, Jag1, Dnajb4, Illb, Spsbl. Btg2, Atf3, Csfl, Trib1. Zbtbl0, Btg1, Rhob, Nfat5. Edn1, Rel. Nr4a3, Nfkbl, Serpinel, Ccl20, Perl, Cxcl2, Map3k8, Trafi, and/or increasing the expression and/or activity of one or more (e.g., 1, 2, 3, 4, or 5) of: Ehdl, Snn, Tnfaip8, Ackr3, Id2, Ccnl, Efnal, Ccndl, Cdknla, Pnrcl (in cases where the component inhibits NF-fB signaling).

In some embodiments, reducing the pro-fibrotic response comprises reducing PDGF signaling. In some embodiments, reducing the pro-fibrotic response comprises reducing the expression and/or activity of one or more (e.g., 1, 2, 3, 4, or 5) of: Pik3rl, Pdgfra, Nfkbia, Pik3cg, Pla2g4a, Tiam1, Pdgfb, Nfkbl, and/or increasing the expression and/or activity of one or more (e.g., 1, 2, 3, 4, or 5) of: Hras (in cases where the component inhibits PDGF signaling).

In some embodiments, the expression and/or activity of NF-cB and/or PDGF expression and/or activity, is reduced by about 25% to about 99% after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between), relative to the expression and/or activity prior to administration of an endothelin receptor antagonist, or a pharmaceutically acceptable salt thereof, and the APRIL binding antibody or antigen-binding fragment thereof. In some embodiments, the expression and/or activity of NF-cB and/or PDGF is reduced by about 25% to about 50%, about 40% to about 60%, about 50% to about 75%, about 60% to about 80%, about 75% to about 90%, about 85% to about 99%, or any value in between.

In some embodiments, reducing the pro-fibrotic response in the mesangial cells comprises reducing matrix secretion by mesangial cells. In some embodiments, reducing matrix secretion by mesangial cells comprises reducing expression and/or activity of one or more of excess matrix secretion by mesangial cells.

Some embodiments provide a method of reducing activation of a mesangial cell in contact with an IgA immune complex, comprising contacting a mesangial cell with an endothelin receptor antagonist, or a pharmaceutically acceptable salt thereof, and an APRIL binding antibody or an antigen-binding fragment thereof. In some embodiments, reducing activation of a mesangial cell comprises reducing expression and/or activity of one or more biomarkers indicative of mesangial cell proliferation.

In some embodiments, reducing activation of a mesangial cell comprises reducing mesangial cell inflammation. In some embodiments, reducing mesangial cell inflammation comprises reducing expression and/or activity of one or more of IL-6, MCP1, or other biomarkers indicative of mesangial cell inflammation.

In some embodiments, reducing activation of a mesangial cell comprises reducing the pro-fibrotic response in the mesangial cells. In some embodiments, reducing the pro-fibrotic response in the mesangial cells comprises reducing expression and/or activity of one or more of TGF, PDGF, CTGF, MMP. TIMPS, or other biomarkers indicative of mesangial cell fibrosis.

In some embodiments, reducing the pro-fibrotic response in the mesangial cells comprises reducing matrix secretion by mesangial cells. In some embodiments, reducing matrix secretion by mesangial cells comprises reducing expression and/or activity of one or more biomarkers indicative of excess matrix secretion by mesangial cells.

In some embodiments, the reducing activation of a mesangial cell comprises reducing undesired mesangial cell migration. In some embodiments, the reduction in undesired mesangial cell migration occurs after about 15 days to about 30 days after treatment with an endothelin receptor antagonist, or a pharmaceutically acceptable salt thereof, and the APRIL binding antibody or antigen-binding fragment thereof. In some embodiments, the reduction in undesired mesangial cell migration occurs after about 3 months to about 6 months after treatment with an endothelin receptor antagonist, or a pharmaceutically acceptable salt thereof, and the APRIL binding antibody or antigen-binding fragment thereof.

In some embodiments, the reducing activation of a mesangial cell comprises reducing undesired mesangial cell proliferation. In some embodiments, the reduction in undesired mesangial cell proliferation occurs after about 15 days to about 30 days after treatment with an endothelin receptor antagonist, or a pharmaceutically acceptable salt thereof, and the APRIL binding antibody or antigen-binding fragment thereof. In some embodiments, the reduction in undesired mesangial cell proliferation occurs after about 3 months to about 6 months after treatment with an endothelin receptor antagonist, or a pharmaceutically acceptable salt thereof, and the APRIL binding antibody or antigen-binding fragment thereof.

In some embodiments, the undesired mesangial cell proliferation is reduced by about 25% to about 99% after treatment with an endothelin receptor antagonist, or a pharmaceutically acceptable salt thereof, and the APRIL binding antibody or antigen-binding fragment thereof (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In some embodiments, the undesired mesangial cell proliferation is reduced by about 25% to about 50%, about 40% to about 60%, about 50% to about 75%, about 60% to about 80%, about 75% to about 90%, about 85% to about 99%, or any value in between.

In some embodiments, mesangial cell activation can be assessed by one or more of serum analysis, urinalysis, and microscopy of a kidney biopsy sample (e.g., light microscopy and/or immunofluorescence microscopy).

In some embodiments, the contacting occurs in vitro. In some embodiments, the contacting occurs in vivo.

Some embodiments provide a method of treating IgA nephropathy in a subject in need thereof, comprising: a) determining that the subject has elevated serum Gd-IgAQ1 levels; and b) administering an endothelin receptor antagonist, or a pharmaceutically acceptable salt thereof, and an APRIL binding antibody or an antigen-binding fragment thereof, to the subject.

Some embodiments provide a method of treating IgA nephropathy in a subject in need thereof, comprising: a) determining that the subject has elevated levels of mesangial activation; and b) administering an endothelin receptor antagonist, or a pharmaceutically acceptable salt thereof, and an APRIL binding antibody or an antigen-binding fragment thereof, to the subject.

In some embodiments, determining of elevated levels of mesangial activation comprises obtaining a sample from the subject and assessing the level of mesangial activation in the same. In some embodiments, the sample is a kidney biopsy sample. In some embodiments, the sample is selected from a blood sample, a urine sample, a kidney biopsy sample, or a combination of two or three of the foregoing.

In some embodiments, the sample exhibits elevated levels of one or more of: matrix secretion by the mesangial cells, IgA-immune complex deposition, mesangial cell proliferation, and endocapillary cell proliferation. In some embodiments, the sample exhibits elevated levels of IgA-immune complex deposition.

In some embodiments, the subject has been determined to have proteinuria of at least about 1 g/day in at least two of three consecutive readings over the year prior to administration of an endothelin receptor antagonist, or a pharmaceutically acceptable salt thereof, and an APRIL binding antibody or an antigen-binding fragment thereof. For example, about 1 g/day, about 1.2 g/day, about 1.4 g/day, about 1.6 g/day, about 1.8 g/day, or about at least 2 g/day.

In some embodiments, the subject has been administered a maximally tolerated stable dose of a RAS inhibitor for at least 12 weeks prior to administration of an endothelin receptor antagonist, or a pharmaceutically acceptable salt thereof, and an APRIL binding antibody or an antigen-binding fragment thereof. In some embodiments, the subject is concurrently administered a maximally tolerated stable dose of a RAS inhibitor and an endothelin receptor antagonist, or a pharmaceutically acceptable salt thereof, and an APRIL binding antibody or an antigen-binding fragment thereof. In some embodiments, the RAS inhibitor is an angiotensin-converting enzyme inhibitor. In some embodiments, the RAS inhibitor is an angiotensin receptor blocker (ARB).

In some embodiments, the subject has been determined to have hematuria prior to administration of an endothelin receptor antagonist, or a pharmaceutically acceptable salt thereof, and an APRIL binding antibody or an antigen-binding fragment thereof. In some embodiments, the hematuria is microhematuria. In some embodiments, the hematuria is gross hematuria.

In some embodiments, the subject has been determined to have an eGFR of at least 30 mL/min/1.73 m² prior to administration of an endothelin receptor antagonist, or a pharmaceutically acceptable salt thereof, and an APRIL binding antibody or an antigen-binding fragment thereof. In some embodiments, the subject has been determined to have an eGFR of about 30 mL/min/1.73 m² to about 60 mL/min/1.73 m² prior to administration of an endothelin receptor antagonist, or a pharmaceutically acceptable salt thereof, and an APRIL binding antibody or an antigen-binding fragment thereof.

Some embodiments provide a method of treating IgA nephropathy in a subject in need thereof, comprising: a) determining that the subject has elevated levels of IgA-immune complexes in the kidney; and b) administering an endothelin receptor antagonist, or a pharmaceutically acceptable salt thereof, and an APRIL binding antibody or an antigen-binding fragment thereof, to the subject.

In some embodiments, determining of elevated levels of IgA-immune complexes in the kidney comprises obtaining a sample from the subject and assessing the level of IgA-immune complexes in the same. In some embodiments, the sample is a kidney biopsy sample. In some embodiments, the sample is selected from a blood sample, a urine sample, a kidney biopsy sample, or a combination of two or three of the foregoing. In some embodiments, the IgA-immune complexes are deposited in the mesangium.

In some embodiments, the levels of IgA-immune complexes can be assessed by one or more of serum analysis, urinalysis, and microscopy of a kidney biopsy sample (e.g., light microscopy and/or immunofluorescence microscopy).

In some embodiments, the sample exhibits elevated levels of one or more of: matrix secretion by the mesangial cells, IgA-immune complex deposition in the mesangium, mesangial cell activation, mesangial cell proliferation, and endocapillary cell proliferation.

In some embodiments, the subject has been determined to have proteinuria of at least about 1 g/day in at least two of three consecutive readings over the year prior to administration of an endothelin receptor antagonist, or a pharmaceutically acceptable salt thereof, and an APRIL binding antibody or an antigen-binding fragment thereof. For example, about 1 g/day, about 1.2 g/day, about 1.4 g/day, about 1.6 g/day, about 1.8 g/day, or about at least 2 g/day.

In some embodiments, the subject has been administered a maximally tolerated stable dose of a RAS inhibitor for at least 12 weeks prior to administration of an endothelin receptor antagonist, or a pharmaceutically acceptable salt thereof, and an APRIL binding antibody or an antigen-binding fragment thereof. In some embodiments, the subject is concurrently administered a maximally tolerated stable dose of a RAS inhibitor and an endothelin receptor antagonist, or a pharmaceutically acceptable salt thereof, and an APRIL binding antibody or an antigen-binding fragment thereof. In some embodiments, the RAS inhibitor is an angiotensin-converting enzyme inhibitor. In some embodiments, the RAS inhibitor is an angiotensin receptor blocker (ARB).

In some embodiments, the subject has been determined to have hematuria prior to administration of an endothelin receptor antagonist, or a pharmaceutically acceptable salt thereof, and an APRIL binding antibody or an antigen-binding fragment thereof. In some embodiments, the hematuria is microhematuria. In some embodiments, the hematuria is gross hematuria.

In some embodiments, the subject has been determined to have an eGFR of at least 30 mL/min/1.73 m² prior to administration of an endothelin receptor antagonist, or a pharmaceutically acceptable salt thereof, and an APRIL binding antibody or an antigen-binding fragment thereof. In some embodiments, the subject has been determined to have an eGFR of about 30 mL/min/1.73 m² to about 60 mL/min/1.73 m² prior to administration of an endothelin receptor antagonist, or a pharmaceutically acceptable salt thereof, and an APRIL binding antibody or an antigen-binding fragment thereof.

In some embodiments, the methods include determining, in the subject, expression and/or activity of one or more of ET1, TGF, PDGF, CTGF, MMP, TIMPS, IGF1, DPEP1, ASL, AMN, ALPL, SLC6A19, IL-6, and NF-kB. In some embodiments, expression and/or activity are determined prior to administration of an endothelin receptor antagonist, or a pharmaceutically acceptable salt thereof, and an APRIL binding antibody or an antigen-binding fragment thereof. In some embodiments, expression and/or activity are determined after administration of an endothelin receptor antagonist, or a pharmaceutically acceptable salt thereof, and an APRIL binding antibody or an antigen-binding fragment thereof.

In some embodiments, the determining the expression and/or activity is performed prior to administration of an endothelin receptor antagonist, or a pharmaceutically acceptable salt thereof, and an APRIL binding antibody or an antigen-binding fragment thereof. In some embodiments, the determining the expression and/or activity is performed after administration of an endothelin receptor antagonist, or a pharmaceutically acceptable salt thereof, and an APRIL binding antibody or an antigen-binding fragment thereof, for example, after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between.

In some embodiments, the subject has been determined to have elevated expression and/or activity of one or more of ET1. TGF, PDGF, CTGF, MMP. TIMPS, IGF1, DPEP1, ASL, AMN, ALPL, SLC6A19, IL-6, NF-kB, PKC, PI3K, Src, Ras, ERK1/2, Rho, Rac, Akt, mTOR, NAPDH oxidase, MAPK, cPLA2, TNF-α, IL-1, CAM, COX-2, iNOS, JAK, STAT3, PI3K, Akt/PKB, IKKs, IkBs, NF-kB, MAPK, Ras, Raf, MEK, ERK, MCP1, Cntfr, Illb, Csfl, Il2ra. Map3k8. Illr1, Pfkfb3, Nr4al, Gem, Fosl2, Klf4, F3, Nfkbia, Ifit2, Nr4a2, Klf2. Jag1. Dnajb4, Illb, Spsbl, Btg2, Atf3, Csfl. Trib1, Zbtb10. Btg1. Rhob, Nfat5, Edn1. Rel, Nr4a3, Nfkbl, Serpinel, Ccl20, Perl, Cxcl2, Map3k8, Trafl, Pik3rl, Pdgfra, Nfkbia, Pik3cg, Pla2g4a, Tiaml, and Pdgfb. In some embodiments, the subject has been determined to have elevated expression and/or activity of one or more of ET1, TGF, PDGF, CTGF, MMP, TIMPS, IGF1. DPEP1, ASL, AMN, ALPL, SLC6A19, IL-6, NF-kB, PKC. PI3K, Src. Ras, ERK1/2, Rho, Rac, Akt, mTOR, NAPDH oxidase, MAPK, cPLA2, TNF-α, IL-1, CAM, COX-2, iNOS, JAK, STAT3, PI3K, Akt/PKB, IKKs, IkBs, NF-kB, MAPK, Ras, Raf, MEK, ERK, and MCP1. In some embodiments, the subject has been determined to have elevated expression and/or activity of one or more of Cntfr, Illb, Csfl, Il2ra, Map3k8, Illr1, Pfkfb3, Nr4a1, Gem, Fosl2, Klf4, F3, Nfkbia, Ifit2, Nr4a2, Klf2, Jag1, Dnajb4, Illb, Spsbl, Btg2, Atf3, Csfl, Trib1, Zbtb10, Btg1, Rhob, Nfat5, Edn1, Rel, Nr4a3, Nfkbl, Serpinel, Ccl20, Perl, Cxcl2, Map3k8, Trafl, Pik3rl, Pdgfra, Nfkbia, Pik3cg, Pla2g4a, Tiaml, and Pdgfb. In some embodiments, the subject has been determined to have elevated expression and/or activity of one or more of ET1, TGF. PDGF, CTGF, MMP, TIMPS. IGF1, DPEPI. ASL. AMN, ALPL, SLC6A19, IL-6, and NF-kB. In some embodiments, the subject has been determined to have elevated expression and/or activity of one or more of ET1, TGF, PDGF, CTGF, MMP, TIMPS, IGF1, DPEP1, ASL, AMN, ALPL, and SLC6A19.

Some embodiments provide a method of treating IgA nephropathy in a subject, comprising: (a) determining that the subject has elevated expression and/or activity of one or more of ET1, TGF, PDGF, CTGF, MMP, TIMPS, IGF1, DPEP1, ASL, AMN, ALPL, SLC6A19, IL-6. NF-kB, PKC, PI3K, Src, Ras, ERK1/2, Rho, Rac, Akt, mTOR, NAPDH oxidase, MAPK, cPLA2, TNF-α, IL-1, CAM, COX-2, iNOS, JAK, STAT3, PI3K, Akt/PKB, IKKs, IkBs, NF-kB, MAPK, Ras, Raf, MEK, ERK, MCP1, Cntfr, Illb, Csfl, Il2ra, Map3k8, Illr1, Pfkfb3, Nr4al, Gem, Fosl2, Klf4, F3, Nfkbia, Ifit2, Nr4a2, Klf2, Jag1, Dnajb4, Illb, Spsbl, Btg2, Atf3, Csfl, Trib1, Zbtb10, Btg1. Rhob, Nfat5, Edn1, Rel, Nr4a3, Nfkbl, SerpineI, Ccl20. Perl, Cxcl2, Map3k8, Trafl, Pik3rl, Pdgfra, Nfkbia. Pik3cg. Pla2g4a. TiamI, and Pdgfb; and (b) administering to the subject an endothelin receptor antagonist, or a pharmaceutically acceptable salt thereof, and an APRIL binding antibody or an antigen-binding fragment thereof.

Some embodiments provide a method of treating IgA nephropathy in a subject determined to have elevated expression and/or activity of one or more of ET1, TGF, PDGF, CTGF, MMP, TIMPS, IGF1, DPEP1, ASL, AMN, ALPL, SLC6A19, IL-6, NF-kB, PKC, PI3K, Src, Ras, ERK1/2, Rho, Rac, Akt, mTOR, NAPDH oxidase, MAPK, cPLA2, TNF-α, IL-1, CAM. COX-2, iNOS, JAK, STAT3. PI3K, Akt/PKB, IKKs, IkBs, NF-kB, MAPK, Ras, Raf, MEK, ERK. MCP1, Cntfr, Illb, Csfl, Il2ra, Map3k8, Illr1, Pfkfb3, Nr4al, Gem, Fosl2, Klf4, F3, Nfkbia, Ifit2, Nr4a2, Klf2, Jag1, Dnajb4, Illb, Spsbl, Btg2, Atf3, Csfl, Trib1, Zbtb10, Btg1, Rhob, Nfat5, Edn1, Rel, Nr4a3, Nfkbl, Serpinel, Ccl20, Pert, Cxcl2, Map3k8, Trafl, Pik3rl, Pdgfra, Nfkbia, Pik3cg, Pla2g4a. Tiaml, and Pdgfb, comprising administering to the subject an endothelin receptor antagonist, or a pharmaceutically acceptable salt thereof, and an APRIL binding antibody or an antigen-binding fragment thereof.

In any of the embodiments described herein, various combinations of an endothelin receptor antagonist, or a pharmaceutically acceptable salt thereof, and an APRIL binding antibody or an antigen-binding fragment thereof, producing an effect, are contemplated. In some embodiments, the effect, for example, any of the beneficial or desired results as described herein, is greater than the sum of the effect observed when the same amount of the endothelin receptor antagonist, or a pharmaceutically acceptable salt thereof, and the APRIL binding antibody or antigen-binding fragment thereof, when co-administered, are administered as a monotherapy. In some embodiments, the co-administration of an endothelin receptor antagonist, or a pharmaceutically acceptable salt thereof, and the APRIL binding antibody or antigen-binding fragment thereof, produce an effect, for example, a therapeutic effect using a smaller dose of either, or both, of the compounds as a monotherapy. For example, producing a therapeutic effect using a smaller dose of an endothelin receptor antagonist, or a pharmaceutically acceptable salt thereof, and the APRIL binding antibody or antigen-binding fragment thereof, compared to the amount used in monotherapy. For example, in some embodiments, the dose of an endothelin receptor antagonist, or a pharmaceutically acceptable salt thereof, administered in combination with the APRIL binding antibody or antigen-binding fragment thereof, may be about 50% to about 90% of the dose of an endothelin receptor antagonist, or a pharmaceutically acceptable salt thereof, administered as a monotherapy to produce the same therapeutic effect, e.g., any of the beneficial or desired results including described herein. In some embodiments, the dose of the APRIL binding antibody or antigen-binding fragment thereof, administered in combination with an endothelin receptor antagonist, or a pharmaceutically acceptable salt thereof, may be about 50% to about 90% of the dose of an APRIL binding antibody or an antigen-binding fragment thereof, administered as a monotherapy to produce the same therapeutic effect. e.g., any of the beneficial or desired results including described herein. For example, treating IgA nephropathy, decreasing renal inflammation and/or fibrosis, decreasing hematuria, decreasing proteinuria, stabilizing eGFR, decreasing the number of IgA-nephropathy associated disease flares, delaying the onset of ESRD, decreasing fatigue, and reducing activation of a mesangial cell.

### C. ATRASENTAN

Atrasentan, also known as (2R,3R,4S)-4-(1,3-benzodioxol-5-yl)-1-[2-(dibutylamino)-2-oxoethyl]-2-(4-methoxyphenyl)pyrrolidine-3-carboxylic acid, ABT-627. A-147627, or A-127722, is a small molecule of the following chemical structure:

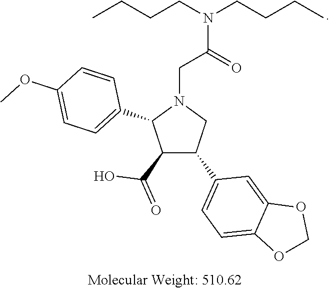 [SMILES: CCCCN(CCCC)C(=O)CN1C[C@H](c2ccc3c(c2)OCO3)[C@@H](C(=O)O)[C@@H]1c1ccc(OC)cc1]

Atrasaentan and methods of preparation thereof are described in U.S. Pat. No. 7,208,517 and PCT Publ. No. WO 1997/030045 (see e.g., Example 501).

In some embodiments, atrasentan is administered as a free base. In some other embodiments, atrasentan is administered as a pharmaceutically acceptable salt as described anywhere herein.

Atrasentan is an ET_{A} inhibitor which is about 1,860 times more selective for ET_{A} relative to ET_{B}. As used herein “ET_{A}” is the abbreviation for endothelin receptor A; and “ET_{B}” is the abbreviation of endothelin receptor B. See, e.g., Ann Rheum Dis., 66(11), pp. 1467-1472 (2007); Eur. Resp. J., 37, pp. 475-476 (2011): Plos One, 9, e87548 (2014); J. Clin. Oncol., 10, 31(14), pp. 1740-7 (2013): Pharmacol. Rev., 68 (2) pp. 357-418 (2016); and Nephrol. Dial. Transplant., 29, pp. i69-i73 (2014).

### Salts

In some embodiments, atrasentan is in the form of a pharmaceutically acceptable salt. The phrase “pharmaceutically acceptable salt” as used herein, refers to pharmaceutically acceptable organic or inorganic salts of a compound of the disclosure (e.g., atrasentan). Exemplary salts include acid addition salts formed by the reaction between atrasentan and an acid (e.g., organic acid or inorganic acid). Non-limiting examples include: sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, mandelate (e.g., (S)-mandelate or (R)-mandelate), gentisinate, fumarate, gluconate, glucuronate, saccharate, formate, benzoate, glutamate, methanesulfonate “mesylate”, ethanesulfonate, benzenesulfonate, and p-toluenesulfonate, pamoate (i.e., 4,4′-methylene-b-s-(2-hydroxy-3-naphthoate)) salts. Exemplary salts also include base addition salts formed by the reaction between atrasentan and a base. Non-limiting examples include alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. When referring to atrasentan, the term “salt” or “salts” is understood to be a salt of atrasentan that can be present alone or in a mixture with free atrasentan.

In some embodiments, atrasentan is in the form of a hydrochloride salt. In some embodiments, the atrasentan hydrochloride has a molar ratio of atrasentan to HCl of 1:1.

In some embodiments, atrasentan is in the form of a mandelate salt. In certain embodiments, atrasentan is in the form of a (S)-mandelate salt. In certain embodiments, atrasentan is in the form of a (R)-mandelate salt. In certain embodiments, in the atrasentan mandelate salt, atrasentan and mandelate has a molar ratio of 1:1. In certain embodiments, in the atrasentan mandelate salt, atrasentan and mandelate has a molar ratio of 2:1. Atrasentan mandelate salt and methods of preparation thereof are further described in U.S. Pat. Nos. 8,962,675 and 9,637,476.

In some embodiments, atrasentan is in the form of a hemisulfate salt. Hemisulfate salt and methods of preparation thereof are further described in U.S. Pat. Nos. 8,962,675 and 9,637,476.

In some embodiments, the atrasentan or a pharmaceutically acceptable salt thereof, is in the form of an anhydrate. In certain embodiments, the atrasentan or a pharmaceutically acceptable salt thereof, is in the form of a hydrate. In certain embodiments, the atrasentan or a pharmaceutically acceptable salt thereof, is in the form of a solvate.

In some embodiments, the endothelin receptor antagonist, or a pharmaceutically acceptable salt thereof, is present in substantially amorphous form (e.g., >75%, >80%, >85%, >90%, >95%, >98%, >99%, or >99.5% amorphous). For example, in some embodiments, the endothelin receptor antagonist, or a pharmaceutically acceptable salt thereof, is amorphous atrasentan hydrochloride (described in PCT Publication No. WO 2006/034085).

In some embodiments, the endothelin receptor antagonist, or a pharmaceutically acceptable salt thereof, is present in one or more crystalline forms (“polymorphs”, e.g., >75%, >80%, >85%, >90%, >95%, >98%, >99%, or >99.5% crystalline). For example, in some embodiments, the endothelin receptor antagonist, or a pharmaceutically acceptable salt thereof, is atrasentan hydrochloride Form 1 (described in PCT Publication No. WO 2006/034094). In some embodiments, the endothelin receptor antagonist, or a pharmaceutically acceptable salt thereof, is atrasentan hydrochloride Form 2 (described in PCT Publication No. WO 2006/034084). In some embodiments, the endothelin receptor antagonist, or a pharmaceutically acceptable salt thereof, is atrasentan hydrochloride Form 3 (described in PCT Publication No. WO 2006/034234 and U.S. Pat. No. 9,051,301).

In certain embodiments, the crystalline atrasentan mandelate salt is a crystalline atrasentan (S)-mandelate salt. In certain embodiments, the atrasentan (S)-mandelate salt is an anhydrous salt. In certain embodiments, the atrasentan (S)-mandelate salt is a solvated salt. In certain embodiments, the atrasentan (S)-mandelate salt is a solvated salt selected from the group consisting of an acetonitrile solvate, an ethanol solvate, and a pyridine solvate. In certain embodiments, the atrasentan (S)-mandelate salt is a hydrated salt.

In certain embodiments, the crystalline atrasentan (S)-mandelate salt is a crystalline atrasentan (S)-mandelate salt wherein the molar ratio of atrasentan to (S)-mandelate is about 1:1. In certain embodiments, the atrasentan (S)-mandelate salt is an anhydrous salt. In certain embodiments, the atrasentan (S)-mandelate salt is a solvated salt. In certain embodiments, the atrasentan (S)-mandelate salt is a solvated salt selected from the group consisting of an acetonitrile solvate, an ethanol solvate, and a pyridine solvate. In certain embodiments, the atrasentan (S)-mandelate salt is a hydrated salt. In certain embodiments, the atrasentan or a pharmaceutically acceptable salt thereof, is substantially a crystalline atrasentan (S)-mandelate salt wherein the molar ratio of atrasentan to (S)-mandelate is about 1:1.

In certain embodiments, the crystalline (S)-mandelate salt has an X-ray powder diffraction pattern comprising peaks at 5.5±0.2, 9.7±0.2, and 19.4±0.2 degrees 2θ when measured at about 25 °C with monochromatic Kil radiation. In certain embodiments, the crystalline (S)-mandelate salt has an X-ray powder diffraction pattern comprising peaks at 5.5 0.2, 9.7±0.2, 12.1±0.2, and 19.4 0.2 degrees 2θ when measured at about 25 °C with monochromatic Kal radiation. In certain embodiments, the crystalline (S)-mandelate salt has an X-ray powder diffraction pattern comprising peaks at 5.5±0.2, 9.7±0.2, 12.1±0.2, 18.0±0.2, 18.4±0.2, and 19.4±0.2 degrees 2θ when measured at about 25 °C with monochromatic Kal radiation. In certain embodiments, the experimental error associated with the X-ray powder diffraction peak values recited in the various embodiments above is +0.1 degrees 2θ. In certain embodiments, the crystalline (S)-mandelate salt is an anhydrous salt. In certain embodiments, the molar ratio of atrasentan to (S)-mandelate is about 1:1.

In certain embodiments, the crystalline (S)-mandelate salt has an orthorhombic lattice type. In certain embodiments, the crystalline (S)-mandelate salt has a P2₁2₁2₁ space group. In certain embodiments, the crystalline (S)-mandelate salt has unit cell a, b and c values of about 9.954 A, about 11.049 A, and about 30.861 A, respectively. In certain embodiments, the crystalline (S)-mandelate salt has unit cell a, B and y values of about 90°, about 90°, and about 90°, respectively. In certain embodiments, the crystalline (S)-mandelate salt has at least three or more of the following properties: (a) an orthorhombic lattice type, (b) a P2₁2₁2₁ space group, (c) unit cell a, b and c values of about 9.954 A, about 11.049 A, and about 30.861 A, respectively, and/or (d) unit cell a, p and y values of about 90°, about 90°, and about 90°, respectively. In certain embodiments, the crystalline (S)-mandelate salt has: (a) an orthorhombic lattice type, (b) a P2₁2₁2₁ space group, (c) unit cell a, b and c values of about 9.954 A, about 11.049 A, and about 30.861 A, respectively, and (d) unit cell a, p and y values of about 90°, about 90°, and about 90°, respectively. In certain embodiments, the crystalline (S)-mandelate salt is an anhydrous salt. In certain embodiments, the molar ratio of atrasentan to (S)-mandelate is about 1:1.

In certain embodiments, the crystalline (S)-mandelate salt is a crystalline atrasentan (S)-mandelate salt wherein the molar ratio of atrasentan to (S)-mandelate is about 2:1. In certain embodiments, the crystalline atrasentan (S)-mandelate salt is an anhydrous salt. In certain embodiments, the crystalline atrasentan (S)-mandelate salt is a solvated salt. In certain embodiments, the crystalline atrasentan (S)-mandelate salt is a hydrated salt. In certain embodiments, the atrasentan or a pharmaceutically acceptable salt thereof, is substantially a crystalline atrasentan (S)-mandelate salt wherein the molar ratio of atrasentan to (S)-mandelate is about 2:1.

In certain embodiments, the crystalline (S)-mandelate salt has an X-ray powder diffraction pattern comprising peaks at 4.5±0.2, 8.6±0.2, and 18.1±0.2 degrees 2θ when measured at about 25 °C with monochromatic Kil radiation. In certain embodiments, the crystalline (S)-mandelate salt has an X-ray powder diffraction pattern comprising peaks at 4.5 ±0.2, 8.6 z 0.2, 18.1±0.2, and 18.7 0.2 degrees 2θ when measured at about 25 °C with monochromatic Kil radiation. In certain embodiments, the crystalline (S)-mandelate salt has an X-ray powder diffraction pattern comprising peaks at 4.5±0.2, 8.6±0.2, 9.1±0.2, 18.1±0.2, and 18.7±0.2 degrees 2θ when measured at about 25 °C with monochromatic Kal radiation. In certain embodiments, the experimental error associated with the X-ray powder diffraction peak values recited in the various embodiments above is z 0.1 degrees 2θ. In certain embodiments, the crystalline (S)-mandelate salt is an anhydrous salt. In certain embodiments, the crystalline (S)-mandelate salt is a hydrated salt.

In certain embodiments, the crystalline atrasentan mandelate salt is a crystalline atrasentan (R)-mandelate salt. In certain embodiments, the crystalline atrasentan (R)-mandelate salt is an anhydrous salt. In certain embodiments, the crystalline atrasentan (R)-mandelate salt is a solvated salt. In certain embodiments, the crystalline atrasentan (R)-mandelate salt is a hydrated salt.

In certain embodiments, the crystalline atrasentan (R)-mandelate salt is a crystalline atrasentan (R)-mandelate salt wherein the molar ratio of atrasentan to (R)-mandelate is about 1:1. In certain embodiments, the crystalline atrasentan (R)-mandelate salt is an anhydrous salt. In certain embodiments, the crystalline atrasentan (R)-mandelate salt is a solvated salt. In certain embodiments, the crystalline atrasentan (R)-mandelate salt is a hydrated salt. In certain embodiments, the atrasentan or a pharmaceutically acceptable salt thereof, is substantially a crystalline atrasentan (R)-mandelate salt wherein the molar ratio of atrasentan to (R)-mandelate is about 1:1.

In certain embodiments, the crystalline atrasentan (R)-mandelate salt has an X-ray powder diffraction pattern comprising peaks at 5.7±0.2, 11.8±0.2, and 20.9±0.2 degrees 2θ when measured at about 25 °C with monochromatic Kal radiation. In certain embodiments, the crystalline atrasentan (R)-mandelate salt has an X-ray powder diffraction pattern comprising peaks at 5.7 1 0.2, 8.2 z 0.2, 11.8 z 0.2, and 20.9 z 0.2 degrees 2θ when measured at about 25 °C with monochromatic Kal radiation. In certain embodiments, the crystalline atrasentan (R)-mandelate salt has an X-ray powder diffraction pattern comprising peaks at 5.7±0.2, 8.2±0.2, 8.6±0.2, 11.8±0.2, and 20.9±0.2 degrees 2θ when measured at about 25 °C with monochromatic Kal radiation. In certain embodiments, the experimental error associated with the X-ray powder diffraction peak values recited in the various embodiments above is ±0.1 degrees 2θ. In certain embodiments, the crystalline atrasentan (R)-mandelate salt is an anhydrous salt.

In some embodiments, the atrasentan or a pharmaceutically acceptable salt thereof, comprises an amorphous atrasentan mandelate salt. In certain embodiments, the atrasentan or a pharmaceutically acceptable salt thereof, is substantially an amorphous atrasentan mandelate salt.

In certain embodiments, the amorphous atrasentan mandelate salt is amorphous atrasentan (S)-mandelate salt. In certain embodiments, the amorphous atrasentan (S)-mandelate salt is an anhydrous salt. In certain embodiments, the amorphous atrasentan (S)-mandelate salt is a solvated salt. In certain embodiments, the amorphous atrasentan (S)-mandelate salt is a solvated salt selected from the group consisting of an acetonitrile solvate, an ethanol solvate, and a pyridine solvate. In certain embodiments, the amorphous atrasentan (S)-mandelate salt is a hydrated salt. In certain embodiments, in the amorphous atrasentan (S)-mandelate salt, the molar ratio of atrasentan and (S)-mandelate is about 1:1. In certain embodiments, in the amorphous atrasentan (S)-mandelate salt, the molar ratio of atrasentan and (S)-mandelate is about 2:1.

In certain embodiments, the amorphous atrasentan mandelate salt is amorphous atrasentan (R)-mandelate salt. In certain embodiments, the amorphous atrasentan (R)-mandelate salt is an anhydrous salt. In certain embodiments, the amorphous atrasentan (R)-mandelate salt is a solvated salt. In certain embodiments, the amorphous atrasentan (R)-mandelate salt is a solvated salt selected from the group consisting of an acetonitrile solvate, an ethanol solvate, and a pyridine solvate. In certain embodiments, the amorphous atrasentan (R)-mandelate salt is a hydrated salt. In certain embodiments, in the amorphous atrasentan (R)-mandelate salt, the molar ratio of atrasentan and (R)-mandelate is about 1:1. In certain embodiments, in the amorphous atrasentan (R)-mandelate salt, the molar ratio of atrasentan and (R)-mandelate is about 2:1.

Crystalline and amorphous atrasentan mandelate salts are further described in U.S. Pat. Nos. 8,962,675 and 9,637,476.

### D. FORMULATIONS AND KITS

The term “pharmaceutical composition” as used herein is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt, or solvate or solvate of the salt thereof, and a pharmaceutically acceptable carrier.

To prepare pharmaceutical or sterile compositions, an active agent is typically admixed with a pharmaceutically acceptable carrier or excipient, see, e.g., Remington's Pharmaceutical Sciences and U.S. Pharmacopeia: National Formulary, Mack Publishing Company, Easton, PA (1984). Formulations of one or more active agents may be prepared by mixing with physiologically acceptable carriers, excipients, or stabilizers in the form of, e.g., lyophilized powders, slurries, aqueous solutions or suspensions (see, e.g., Hardman, et al., 2001, Goodman and Gilman's The Pharmacological Basis of Therapeutics, McGraw-Hill, New York, NY: Gennaro, 2000, Remington: The Science and Practice of Pharmacy, Lippincott, Williams, and Wilkins, New York, NY; Avis, et al. (eds.), 1993, Pharmaceutical Dosage Forms: Parenteral Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker. Inc., New York, NY).

Some embodiments provide a kit comprising: endothelin receptor antagonist, or a pharmaceutically acceptable salt thereof, and an APRIL binding antibody or an antigen-binding fragment thereof, wherein the endothelin receptor antagonist, or a pharmaceutically acceptable salt thereof, and the APRIL binding antibody or antigen-binding fragment thereof can be in the same and/or separate dosage form. In some embodiments, the kit can further comprise an SGLT-2 inhibitor.

Some embodiments provide a kit comprising: an SGLT-2 inhibitor and an APRIL binding antibody or an antigen-binding fragment thereof, wherein the SGLT-2 inhibitor and the APRIL binding antibody or antigen-binding fragment thereof can be in the same and/or separate dosage form.

### Exemplary Dosage Forms of an APRIL Binding Antibody or an Antigen-Binding Fragment Thereof

Some embodiments provide a sterile liquid formulation of n APRIL binding antibody or an antigen-binding fragment thereof.

Exemplary formulations of an APRIL binding antibody or an antigen-binding fragment thereof and methods of making the same are further described in U.S. Pat. Nos. 9,364,458 and 10,016,393.

### Exemplary Dosage Forms of an Endothelin Receptor Antagonist

In some embodiments, provided herein a stable solid pharmaceutical dosage form comprising an endothelin receptor antagonist and a pharmaceutically acceptable diluent.

Suitable diluents for use in the described dosage forms include, but are not limited to, lactose (such as lactose monohydrate, lactose anhydrous, and PHARMATOSE(R DCL21), sucrose, glucose, mannitol, sorbitol, isomalt, microcrystalline cellulose (such as AVICEL®PH101 and AVICEL®PH102), silicified microcrystalline cellulose (such as PROSOLV®SMCC 50 and SMCC 90), dicalcium phosphate, starches, and combinations thereof. In some embodiments, the diluent is selected from the group consisting of lactose, mannitol, isomalt, microcrystalline cellulose, dicalcium phosphate, and combinations thereof.

In some embodiments, the diluent is lactose.

In some embodiments, the weight percent of the diluent in the dosage form is from about 70 weight percent to about 99 weight percent. In some embodiments, the weight percent of the diluent in the dosage form is from about 80 weight percent to about 99 weight percent. In some embodiments, the weight percent of the diluent in the dosage form is from about 85 weight percent to about 99 weight percent. In certain of the foregoing embodiments, the diluent is selected from the group consisting of lactose, mannitol, isomalt, and combinations thereof. As a non-limiting example, the diluent can be lactose.

In some embodiments, the endothelin receptor antagonist is atrasentan, or a pharmaceutically acceptable salt thereof. In some embodiments, the endothelin receptor antagonist is sparsentan, or a pharmaceutically acceptable saltt thereof.

In some embodiments, provided herein a stable solid pharmaceutical dosage form comprising about 200 mg to about 1,000 mg of sparsentan, or an equivalent amount of a pharmaceutically acceptable salt thereof.

In some embodiments, provided herein a stable solid pharmaceutical dosage form comprising: (a) about 0.25 mg to about 1.25 mg of atrasentan, or an equivalent amount of a pharmaceutically acceptable salt thereof; wherein the weight percent of atrasentan, or pharmaceutically acceptable salt thereof, in the dosage form is from about 0.05 weight percent to about 2.0 weight percent on an atrasentan free base equivalent weight basis; and (b) a pharmaceutically acceptable diluent.

In some embodiments, provided herein a stable solid pharmaceutical dosage form comprising: (a) about 0.25 mg to about 1.25 mg of atrasentan, or an equivalent amount of a pharmaceutically acceptable salt thereof; wherein the weight percent of atrasentan, or pharmaceutically acceptable salt thereof, in the dosage form is from about 0.05 weight percent to about 2.0 weight percent on an atrasentan free base equivalent weight basis; (b) a pharmaceutically acceptable anti-oxidant; wherein the molar ratio of the anti-oxidant to atrasentan, or pharmaceutically acceptable salt thereof, is from about 10:1 to about 1:10; and (c) a pharmaceutically acceptable diluent.

The dosage form can comprise a free base of atrasentan, a pharmaceutically acceptable salt of atrasentan, or a combination thereof. In some embodiments, the dosage form comprises a free base of atrasentan. In some embodiments, the dosage form comprises a pharmaceutically acceptable salt of atrasentan. In some embodiments, the dosage form comprises atrasentan hydrochloride. In some embodiments, the dosage form comprises atrasentan hydrochloride having a polymorph form selected from the group consisting of atrasentan hydrochloride Form 1, atrasentan hydrochloride Form 2, and atrasentan hydrochloride Form 3. In some embodiments, the dosage form comprises amorphous atrasentan hydrochloride. In some embodiments, the dosage form comprises atrasentan hydrochloride Form 1. In some embodiments, the dosage form comprises atrasentan hydrochloride Form 2. In some embodiments, the dosage form comprises atrasentan hydrochloride Form 3. In some embodiments, the dosage form comprises atrasentan mandelate. In certain embodiments, the dosage form comprises a crystalline atrasentan mandelate (e.g., a crystalline atrasentan (S)-mandelate and/or a crystalline atrasentan (R)-mandelate). In certain embodiments, the dosage form comprises an amorphous atrasentan mandelate (e.g., an amorphous atrasentan (S)-mandelate and/or an amorphous atrasentan (R)-mandelate). In certain of the foregoing embodiments (when the dosage form comprises a crystalline and/or amorphous atrasentan (S)- and/or (R)-mandelate), the molar ratio of atrasentan and mandelate is 1:1. In certain other embodiments, the molar ratio of atrasentan and mandelate is 2:1.

In some embodiments, the dosage form comprises from about 0.25 mg to about 1.25 mg of atrasentan, or an equivalent amount of a pharmaceutically acceptable salt thereof. In some embodiments, the dosage form comprises from about 0.40 mg to about 1.00 mg of atrasentan, or an equivalent amount of a pharmaceutically acceptable salt thereof. In some embodiments, the dosage form comprises from about 0.40 mg to about 0.85 mg of atrasentan, or an equivalent amount of a pharmaceutically acceptable salt thereof. In some embodiments, the dosage form comprises about 0.50 mg of atrasentan, or an equivalent amount of a pharmaceutically acceptable salt thereof. In some embodiments, the dosage form comprises about 0.75 mg of atrasentan, or an equivalent amount of a pharmaceutically acceptable salt thereof.

In some embodiments, the dosage form comprises from about 0.25 mg to about 1.25 mg of atrasentan, or an equivalent amount of a pharmaceutically acceptable salt thereof. In some embodiments, the dosage form comprises from about 0.40 mg to about 1.00 mg of atrasentan, or an equivalent amount of atrasentan hydrochloride. In some embodiments, the dosage form comprises from about 0.40 mg to about 0.85 mg of atrasentan, or an equivalent amount of atrasentan hydrochloride. In some embodiments, the dosage form comprises about 0.50 mg of atrasentan, or an equivalent amount of atrasentan hydrochloride. In some embodiments, the dosage form comprises about 0.75 mg of atrasentan, or an equivalent amount of atrasentan hydrochloride.

In some embodiments, the dosage form further comprises a pharmaceutically acceptable disintegrant and the weight to weight ratio of the disintegrant to the anti-oxidant (e.g., L-cysteine), or pharmaceutically acceptable salt or ester thereof, is from about 60:1 to about 3:1. In some embodiments, the weight to weight ratio of the disintegrant to the anti-oxidant (e.g., L-cysteine), or pharmaceutically acceptable salt or ester thereof, is from about 50:1 to about 4:1. In some embodiments, the weight to weight ratio of the disintegrant to the anti-oxidant (e.g., L-cysteine), or pharmaceutically acceptable salt or ester thereof, is from about 35:1 to about 5:1.

Suitable anti-oxidants for use in the described dosage forms include anti-oxidants that function as reducing agents and are oxidized to pharmaceutically acceptable reduced products in the dosage form. In some embodiments, the anti-oxidant has an oxidation reduction potential less than the oxidation reduction potential of atrasentan (i.e., an oxidation reduction potential less than about 900 mV) and greater than about 550 mV. In some embodiments, the anti-oxidant has an oxidation reduction potential less than about 550 mV. In some embodiments, the anti-oxidant has an oxidation reduction potential from about 1 mV to about 550 mV. In some embodiments, the solubility of the anti-oxidant in water at about 25 °C is greater than about 24 mg/mL. In some embodiments, the anti-oxidant is an amino acid, or a pharmaceutically acceptable salt or ester thereof. In some embodiments, the anti-oxidant is cysteine. In some embodiments, the anti-oxidant is L-cysteine, or a pharmaceutically acceptable salt or ester thereof. In some embodiments, the anti-oxidant is selected from the group consisting of L-cysteine hydrochloride monohydrate, L-cysteine hydrochloride anhydrate, and L-cysteine ethyl ester. In some embodiments, the dosage form comprises L-cysteine hydrochloride monohydrate.

In some embodiments, the weight percent of the anti-oxidant in the dosage form is from about 0.05 weight percent to about 1.0 weight percent. In some embodiments, the weight percent of the anti-oxidant in the dosage form is from about 0.07 weight percent to about 0.7 weight percent. In some embodiments, the weight percent of the anti-oxidant in the dosage form is from about 0.09 weight percent to about 0.5 weight percent.

In some embodiments, the molar ratio of the anti-oxidant to atrasentan, or pharmaceutically acceptable salt thereof, is from about 10:1 to about 1:10. In some embodiments, the molar ratio of the anti-oxidant to atrasentan, or pharmaceutically acceptable salt thereof, in the dosage form is from about 5:1 to about 1:5. In some embodiments, the molar ratio of the anti-oxidant to atrasentan, or pharmaceutically acceptable salt thereof, is from about 2:1 to about 1:2. In some embodiments, the molar ratio of the anti-oxidant to atrasentan, or pharmaceutically acceptable salt thereof, is about 1:1.

In some embodiments, the anti-oxidant is L-cysteine, or a pharmaceutically acceptable salt thereof. In certain embodiments, the weight percent of the L-cysteine, or pharmaceutically acceptable salt or ester thereof, in the dosage form is from about 0.05 weight percent to about 1.0 weight percent. In certain embodiments, the weight percent of the L-cysteine, or pharmaceutically acceptable salt or ester thereof, in the dosage form is from about 0.07 weight percent to about 0.7 weight percent. In certain embodiments, the weight percent of the L-cysteine, or pharmaceutically acceptable salt or ester thereof, in the dosage form is from about 0.09 weight percent to about 0.5 weight percent.

In some embodiments, the dosage form is a solid pharmaceutical dosage form comprising from about 0.25 mg to about 1.25 mg of atrasentan or a pharmaceutically acceptable salt thereof (e.g., atrasentan hydrochloride) on an atrasentan parent equivalent weight basis. In some embodiments, the dosage form is a solid pharmaceutical dosage form comprising from about 0.40 mg to about 1.00 mg of atrasentan or a pharmaceutically acceptable salt thereof (e.g., atrasentan hydrochloride) on an atrasentan parent equivalent weight basis. In some embodiments, the dosage form is a solid pharmaceutical dosage form comprising about 0.50 mg of atrasentan or a pharmaceutically acceptable salt thereof (e.g., atrasentan hydrochloride) on an atrasentan parent equivalent weight basis. In some embodiments, the dosage form is a solid pharmaceutical dosage form comprising about 0.75 mg of atrasentan or a pharmaceutically acceptable salt thereof (e.g., atrasentan hydrochloride) on an atrasentan parent equivalent weight basis.

In certain of the foregoing embodiments, the dosage form is a tablet.

Some embodiments provide a kit comprising (a) a pharmaceutical composition comprising an endothelin receptor antagonist, or a pharmaceutically acceptable salt thereof, (b) an APRIL binding antibody or an antigen-binding fragment thereof; and (c) instructions for use.

### E. DOSAGE AND ADMINISTRATION

In some embodiments, the dose of the APRIL binding antibody or antigen-binding fragment thereof is about 4 mg/kg to about 15 mg/kg, for example, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 10.5 mg/kg, about 11 mg/kg, about 11.5 mg/kg, about 12 mg/kg, about 12.5 mg/kg, about 13 mg/kg, about 13.5 mg/kg, about 14 mg/kg, about 14.5 mg/kg, or about 15 mg/kg. In some embodiments, the dose of the APRIL binding antibody or antigen-binding fragment thereof is about 4 mg/kg to about 10 mg/kg. In some embodiments, the dose of the APRIL binding antibody or antigen-binding fragment thereof is about 8 mg/kg to about 12 mg/kg. In some embodiments, the dose of the APRIL binding antibody or antigen-binding fragment thereof is about 10 mg/kg to about 15 mg/kg.

In some embodiments, the total dose of the APRIL binding antibody or antigen-binding fragment thereof is about 450 mg to about 600 mg. In some embodiments, the total dose of the APRIL binding antibody or antigen-binding fragment thereof is 450 mg or 600 mg.

In some embodiments, the APRIL binding antibody or antigen-binding fragment thereof is administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrastemal injection and infusion. In some embodiments, the route of administration of the APRIL binding antibody or antigen-binding fragment thereof is intravenous injection or infusion. In some embodiments, the route of administration of the APRIL binding antibody or antigen-binding fragment thereof is intravenous infusion. In some embodiments, the route of administration of the APRIL binding antibody or antigen-binding fragment thereof is intravenous injection. In some embodiments, the route of administration of the APRIL binding antibody or antigen-binding fragment thereof is subcutaneous injection.

In some embodiments, the APRIL binding antibody or antigen-binding fragment thereof is administered once per week. In some embodiments, the APRIL binding antibody or antigen-binding fragment thereof is administered every other week (i.e., once every two weeks). In some embodiments, the APRIL binding antibody or antigen-binding fragment thereof is administered once every three weeks. In some embodiments, the APRIL binding antibody or antigen-binding fragment thereof is administered once per month.

In some embodiments, the endothelin receptor antagonist, or a pharmaceutically acceptable salt thereof, is administered at the approved dose of the endothelin receptor antagonist, or a pharmaceutically acceptable salt thereof.

In some embodiments, the endothelin receptor antagonist, or a pharmaceutically acceptable salt thereof, is sparsentan, or a pharmaceutically acceptable salt thereof, and the sparsentan is administered at a dose of about 200 mg to about 1,000 mg, or an equivalent amount of a pharmaceutically acceptable salt thereof. In some embodiments, the sparsentan is administered at a dose of about 200 mg to about 600 mg, or an equivalent amount of a pharmaceutically acceptable salt thereof. In some embodiments, the sparsentan is administered at a dose of about 400 mg to about 800 mg, or an equivalent amount of a pharmaceutically acceptable salt thereof. In some embodiments, the sparsentan is administered at a dose of 200 mg, 400 mg, or 600 mg, or an equivalent amount of a pharmaceutically acceptable salt thereof.

In some embodiments, the endothelin receptor antagonist, or a pharmaceutically acceptable salt thereof, is atrasentan, or a pharmaceutically acceptable salt thereof, and the atrasentan is administered at a dose of from about 0.10 mg to about 1.50 mg (e.g. about 0.10, about 0.20, about 0.30, about 0.40, about 0.50, about 0.60, about 0.70, about 0.75, about 0.80, about 0.90, about 1.00, about 1.10, about 1.20, about 1.30, about 1.40, about 1.50, or any value in between) of atrasentan or an equivalent amount of a pharmaceutically acceptable salt thereof. In certain embodiments, the dose of atrasentan is about 0.75 mg, or an equivalent amount of a pharmaceutically acceptable salt thereof. In certain embodiments, the dose of atrasentan is about 0.25 mg, or an equivalent amount of a pharmaceutically acceptable salt thereof. In certain embodiments, the dose of atrasentan is about 0.35 mg, or an equivalent amount of a pharmaceutically acceptable salt thereof. In certain embodiments, the dose of atrasentan is about 1.00 mg, or an equivalent amount of a pharmaceutically acceptable salt thereof. In certain embodiments, the dose of atrasentan is about 1.25 mg, or an equivalent amount of a pharmaceutically acceptable salt thereof. In certain embodiments, the dose of atrasentan is about 1.50 mg, or an equivalent amount of a pharmaceutically acceptable salt thereof.

In some embodiments, the endothelin receptor antagonist, or a pharmaceutically acceptable salt thereof, is administered orally, e.g., as a tablet or capsule.

In some embodiments, the endothelin receptor antagonist, or a pharmaceutically acceptable salt thereof, is administered once per day. In some embodiments, the endothelin receptor antagonist, or a pharmaceutically acceptable salt thereof, is administered every other day.

In some embodiments, the endothelin receptor antagonist, or a pharmaceutically acceptable salt thereof, is administered more than once per day, such as in a divided dose. In some embodiments, the endothelin receptor antagonist, or a pharmaceutically acceptable salt thereof, is administered once per day. For example, in some embodiments, atrasentan, or a pharmaceutically acceptable salt thereof, is administered to the subject once per day. In some embodiments, atrasentan, or a pharmaceutically acceptable salt thereof, is administered to the subject at a dose of about 0.75 mg of atrasentan free base once per day. In some embodiments, atrasentan, or a pharmaceutically acceptable salt thereof, is administered to the subject at a dose of 0.75 mg of atrasentan free base once per day. In some embodiments, atrasentan, or a pharmaceutically acceptable salt thereoft is administered to the subject at a dose of about 0.25 mg of atrasentan free base once per day. In some embodiments, atrasentan, or a pharmaceutically acceptable salt thereof, is administered to the subject at a dose of 0.25 mg of atrasentan free base once per day. In some embodiments, atrasentan, or a pharmaceutically acceptable salt thereof, is administered to the subject at a dose of about 0.35 mg of atrasentan free base once per day. In some embodiments, atrasentan, or a pharmaceutically acceptable salt thereof, is administered to the subject at a dose of 0.35 mg of atrasentan free base once per day. In some embodiments, atrasentan, or a pharmaceutically acceptable salt thereof, is administered to the subject at a dose of about 1.00 mg of atrasentan free base once per day. In some embodiments, atrasentan, or a pharmaceutically acceptable salt thereof, is administered to the subject at a dose of 1.00 mg of atrasentan free base once per day. In some embodiments, atrasentan, or a pharmaceutically acceptable salt thereof, is administered to the subject at a dose of about 1.50 mg of atrasentan free base once per day. In some embodiments, atrasentan, or a pharmaceutically acceptable salt thereof, is administered to the subject at a dose of 1.50 mg of atrasentan free base once per day.

In some embodiments, the endothelin receptor antagonist, or a pharmaceutically acceptable salt thereof, is administered in a therapeutically effective amount. In some embodiments, the APRIL binding antibody or antigen-binding fragment thereof is administered in a therapeutically effective amount. In some embodiments, the endothelin receptor antagonist, or a pharmaceutically acceptable salt thereof, and the APRIL binding antibody or antigen-binding fragment thereof, when administered together are provided in a therapeutically effective amount.

Toxicity and therapeutic efficacy of the active agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD50 and ED50. The data obtained from these cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

Suitable routes of administration include parenteral administration, such as intramuscular, intravenous, or subcutaneous administration and oral administration. Administration of antibodies, used in the pharmaceutical composition or to practice the method of the present disclosure can be carried out in a variety of conventional ways, such as oral ingestion, inhalation, topical application or cutaneous, subcutaneous, intraperitoneal, parenteral, intraarterial or intravenous injection. In one embodiment, the antibody of the disclosure is administered intravenously. In another embodiment, the antibody of the disclosure is administered subcutaneously. In one embodiment, the endothelin receptor agonists and/or SGLT-2 inhibitors are administered orally.

A preferred dose protocol is one involving the maximal dose or dose frequency of the combination therapy that achieves a desired therapeutic effect (e.g., reducing IgA levels) while avoiding significant undesirable side effects. Dosing of anti-APRIL antibody or antigen-binding fragment thereof as described herein can be about every week, about every two weeks, about every three weeks, about every 4 weeks, about every 8 weeks, etc., either buy intravenous injection, or by subcutaneous injection (e.g., into the thigh, abdomen, upper arm, etc.). The dose per injection or infusion may be about 10 to 1350 mg, e.g. about 50 mg., about 150 mg, about 300 mg, about 450 mg, about 600 mg, about 750 mg, about 1000 mg, or about 1350 mg. In certain embodiments, dosing of anti-APRIL antibody or antigen-binding fragment thereof will be by subcutaneous injection, with a dose per dosing event (where a “dosing event” refers to one or more deliveries, such as injections, intended to provide a single administration to the individual, where the administrations are given in the same or different sites on the individual) of about 600 mg, with a dosing frequency of once every week, or once every two weeks. A preferred formulation for intravenous dosing is an aqueous buffered solution at a concentration of about 15-25 mg/mL, or about 20 mg, while the preferred formulation for subcutaneous dosing is at about 125-175 mg, or about 150 mg. These formulations preferably comprise L-histidine. L-arginine, sorbitol, and polysorbate 20 at pH 6.3±0.2. Preferably the L-histidine is at a concentration of about 8-12 nM, or about 10 mM, the L-arginine is at a concentration of about 60-90 mM, or about 75 mM, the sorbitol is at a concentration of about 2.4-3.6%, or about 3% (w/w), and the polysorbate 20 is at a concentration of about 0.008-0.012%, or about 0.01% (w/w). More preferably the aqueous buffered solution comprises, consists essentially of, or consists of 10 mM L-histidine, 75 mM L-arginine, 3% (w/w) sorbitol and 0.01% (w/w) polysorbate 20 at pH 6.3±0.2. The pH of the aqueous buffered solution can be adjusted to 6.3±0.2 using a suitable sterile acid/base, such as hydrochloric acid and sodium hydroxide. Formulations for intravenous infusion can be diluted in sterile saline (0.9%) prior to infusion, for example the desired amount of the anti-APRIL antibody or antigen-binding fragment thereof can be diluted to a volume of about 250 mL, for example 15 mL of a 20 mg/mL formulation of antibody can be diluted with 235 mL of sterile saline solution prior to infusion of a 300 mg dose. The formulation for subcutaneous injection can be used without further dilution.

The therapeutically effective amount and the frequency of administration of, and the length of treatment with, the APRIL binding antibody or antigen-binding fragment thereof, e.g., BION-1301, described herein to treat an antibody-associated condition may depend on various factors, including the nature and severity of the condition, the potency of the antibody, the mode of administration, the age, body weight, general health, gender and diet of the subject, and the response of the subject to the treatment, and can be determined by the treating physician. The APRIL binding antibody or antigen-binding fragment thereof, e.g., BION-1301, can be administered once daily, once every 2 days, once every 3 days, twice weekly, once weekly, once every 2 weeks, once every 3 weeks, once monthly, once every 6 weeks, once every 2 months or once every 3 months, or as deemed appropriate by the treating physician.

The APRIL binding antibody or antigen-binding fragment thereof, e.g., BION-1301, can be administered over a period of at least about 1 week, 2 weeks, 1 month (4 weeks), 6 weeks, 2 months, 3 months, 6 months, 1 year, 2 years, 3 years or longer, or as deemed appropriate by the treating physician. The APRIL-associated condition can be a chronic condition. A chronic condition can exist for, e.g., at least about 6 weeks, 2 months, a year, or longer. The antibody can be administered over a period of at least about 6 weeks, 2 months, 3 months or 6 months, a year, or even multiple years as required for medical care of an individual.

The APRIL binding antibody or antigen-binding fragment thereof, e.g., BION-1301, can also be administered in an irregular manner to treat an antibody-associated condition. Early achievement of an effective target antibody concentration (a therapeutic dose level) with a loading dose followed by maintenance dosing with the antibody (frontloading) may be more effective than conventional therapy in terms of requiring a lower total antibody dose and faster time to maximum target engagement. As used herein, such an administration protocol is referred to as a “loading/maintenance administration protocol.” An effective target antibody concentration may be reached in 4 weeks or less, preferably 3 weeks or less, more preferably 2 weeks or less, most preferably 1 week or less, including 1 day or less using a loading dose. The target serum concentration is then maintained by administration of an equal or smaller (or less frequent) maintenance dose during the remainder of the treatment regimen or until suppression of disease symptoms is achieved.

When administered active agents are administered in combination, separate dosage forms of the active agents can be administered to the subject or a single dosage form comprising both active agents can be administered to the subject. If administered as a separate dosage form, the therapeutic agents may be administered simultaneously or sequentially (in either order). Administration of two or more agents in combination can also be referred to herein as “co-administration.” Methods for co-administration or treatment with a second therapeutic agent are well known in the art, see, e.g., Hardman, et al. (eds.), 2001, Goodman and Gilman's The Pharmacological Basis of Therapeutics, 10th ed., McGraw-Hill, New York, NY: Poole and Peterson (eds.), 2001, Pharmacotherapeutics for Advanced Practice: A Practical Approach, Lippincott, Williams & Wilkins, Phila., PA; Chabner and Longo (eds.), 2001, Cancer Chemotherapy and Biotherapy, Lippincott, Williams & Wilkins, Phila., PA.

### F. ADDITIONAL COMBINATIONS

The methods of the present disclosure also contemplate treatments comprising administering an endothelin receptor antagonist, or a pharmaceutically acceptable salt thereof, and the APRIL binding antibody or antigen-binding fragment thereof, as described in any of the embodiments of the disclosure, in combination with one or more additional therapeutic agents (such as an inhibitor of one or more elements of the renin-angiotensin-aldosterone system). Accordingly, an endothelin receptor antagonist, or a pharmaceutically acceptable salt thereof, and the APRIL binding antibody or antigen-binding fragment thereof as described anywhere herein can be administered alone or in combination with one or more additional therapeutic agents. When administered in combination with one or more additional therapeutic agents, separate dosage forms can be administered to the subject or a single dosage form comprising both an endothelin receptor antagonist, or a pharmaceutically acceptable salt thereof, and the APRIL binding antibody or antigen-binding fragment thereof, and the additional therapeutic agent(s) can be administered to the subject. If administered as a separate dosage form, the additional therapeutic agent may be administered simultaneously with the atrasentan dosage form of the present disclosure or sequentially (in either order) with the atrasentan dosage form of the present disclosure. Administration of two or more agents in combination can also be referred to herein as “co-administration.”

Representative additional therapeutic agents include, for example, diuretics, antihypertensive agents, therapeutic agents for diabetes or diabetic complications, and therapeutic agents for hyperlipidemia.

In some embodiments, an endothelin receptor antagonist, or a pharmaceutically acceptable salt thereof, and the APRIL binding antibody or antigen-binding fragment thereof may be co-administered with one or more diuretics such as hydrochlorothiazide (such as MICROZIDE™ or ORETIC™), hydroflumethiazide (such as SALURON™), bemetanide (such as BUMEX™), torsemide (such as DEMADEX™) metolazone (such as ZAROXOLYN™), chlorothiazide (such as DIURIL™, ESIDRIX™ or HYDRODIURIL™) triamterene (such as DYRENIUM™), ethacrynic acid (such as EDECRIN™), chlorthalidone (such as HYGROTON™), furosemide (such as LASIX™) indapamide (such as LOZOL™) or amiloride (such as MIDAMOR™ or MODURETIC™).

In some embodiments, an endothelin receptor antagonist, or a pharmaceutically acceptable salt thereof, and the APRIL binding antibody or antigen-binding fragment thereof may be co-administered with one or more thiazide diuretics, such as chlorothiazide, chlorthalidone, hydrochlorothiazide, trichlormethiazide, indapamide, or metolazone.

In some embodiments, an endothelin receptor antagonist, or a pharmaceutically acceptable salt thereof, and the APRIL binding antibody or antigen-binding fragment thereof may be co-administered with one or more loop diuretics, such as bumetanide, ethacrynic acid, furosemide, or torsemide.

In some embodiments, an endothelin receptor antagonist, or a pharmaceutically acceptable salt thereof, and the APRIL binding antibody or antigen-binding fragment thereof may be co-administered with one or more potassium-sparing diuretics, such as amiloride, eplerenone, spironolactone, and triamterene.

In some embodiments, an endothelin receptor antagonist, or a pharmaceutically acceptable salt thereof, and the APRIL binding antibody or antigen-binding fragment thereof may be co-administered with one or more angiotensin converting enzyme (ACE) inhibitors such as quinapril (such as ACCUPRIL™), fosinopril, perindopril (such as ACEON™), captopril (such as CAPOTEN™), enalapril (such as VASOTEC™) ENALAPRILAT™, ramipril (such as ALTACE™), cilazapril, delapril, fosenopril (such as MONOPRIL™), zofenopril, indolapril, benazepril (such as LOTENSIN™), lisinopril (such as PRINIVIL™ or ZESTRIL™), spirapril, trandolapril (such as MAVIK™), perindep, pentopril, moexipril (such as UNIVASC™), pivopril, temocapril, omapatrilat, imidapril, rescinnamine, benazeprilat, fosinoprilat, ramiprilat, perindoprilat, quinaprilat, trandolaprilat, moexiprilat, Quinoline Yellow WS, or cilazaprilat. In certain embodiments, the ACE inhibitor is selected from the group consisting of quinapril, fosinopril perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril.

In some embodiments, an endothelin receptor antagonist, or a pharmaceutically acceptable salt thereof, and the APRIL binding antibody or antigen-binding fragment thereof may be co-administered with one or more angiotensin II receptor blockers (ARB) such as candesartan (such as ATACAND™), candesartan cilexetil, eprosartan (such as TEVETEN™), irbesartan (such as AVEPRO™) losartan (such as COZAAR™), olmesartan, olmesartan medoxomil (such as BENICAR™) tasosartan, telmisartan (such as MICARDIS™) valsartan (such as DIOVAN™), zolasartan, azilsartan medoxomil, F1-6828K, RNH-6270, UR-7198, Way-126227, KRH-594, TAK-536, BRA-657, or TA-606. In certain embodiments, the ARB is selected from the group consisting of candesartan, candesartan cilexetil, eprosartan, irbesartan, losartan, olmesartan, olmesartan medoxomil, telmisartan, valsartan, azilsartan medoxomil, and BRA-657.

In some embodiments, an endothelin receptor antagonist, or a pharmaceutically acceptable salt thereof, and the APRIL binding antibody or antigen-binding fragment thereof may be co-administered with one or more calcium channel blockers such as nifedipine (such as ADALAT™, ADALAT CC™, or PROCARDIA™), verapamil (such as GALAN™, COVERA-HS™, ISOPTIN SR™, or VERELAN™), diltiazem (such as CARDIZEM™, CARDIZEM CD™, CARDIZEM LA™, CARDIZEM SR™, DILACOR™, TIAMATE™, or TIAZAC™), isradipine (such as DYNACIRC™ or DYNACIRC CR™) amlodipine (such as NORVASC™), felodipine (such as PLENDIL™), nisoldipine (such as SULAR™), bepridil (such as VASCOR™), vatanidipine, clevidipine, lercanidipine, or dilitiazem.

In some embodiments, an endothelin receptor antagonist, or a pharmaceutically acceptable salt thereof, and the APRIL binding antibody or antigen-binding fragment thereof may be co-administered with one or more renin inhibitors such as aliskiren (such as TEKTURNA™).

In some embodiments, an endothelin receptor antagonist, or a pharmaceutically acceptable salt thereof, and the APRIL binding antibody or antigen-binding fragment thereof may be co-administered with one or more aldosterone receptor antagonists such as eplerenone (such as INSPRA™) or spironolactone (such as ALDACTONE™).

In some embodiments, an endothelin receptor antagonist, or a pharmaceutically acceptable salt thereof, and the APRIL binding antibody or antigen-binding fragment thereof may be co-administered with one or more alpha blockers such as dozazosin (such as CARDURA™) phenoxybenzamine (such as DIBENZYLINE™), terazosin (such as HYTRIN™), CDRT-93/478, or CR-2991.

In some embodiments, an endothelin receptor antagonist, or a pharmaceutically acceptable salt thereof, and the APRIL binding antibody or antigen-binding fragment thereof may be co-administered with one or more beta blockers such as timolol (such as BLOCARDEN™) carteolol (such as CARTROL™), carvedilol (such as COREG™) nadolol (such as CORGARD™), propranolol (such as INNOPRAN XL™), betaxolol (such as KERLONE™) penbutolol (such as LEVATOL™), metoprolol (such as LOPRESSOR™ or TOPROL-XL™), atenolol (such as TENORMIN™), pindolol (such as VISKEN™), or bisoprolol.

In some embodiments, an endothelin receptor antagonist, or a pharmaceutically acceptable salt thereof, and the APRIL binding antibody or antigen-binding fragment thereof may be co-administered with one or more alpha-beta blockers such as labetalol (such as NORMODYNE™ or TRANDATE™).

In some embodiments, an endothelin receptor antagonist, or a pharmaceutically acceptable salt thereof, and the APRIL binding antibody or n antigen-binding fragment thereof may be co-administered with one or more central antiadrenergics such as methyldopa (such as ALDOMET™), clonidine (such as CATAPRES™ or CATAPRES-TTS™), guanfacine (such as TENEX™), or guanabenz (such as WYTENSIN™).

In some embodiments, an endothelin receptor antagonist, or a pharmaceutically acceptable salt thereof, and the APRIL binding antibody or antigen-binding fragment thereof may be co-administered with one or more glycosides/inotropic agents such as digoxin (such as LANOXIN™).

In some embodiments, an endothelin receptor antagonist, or a pharmaceutically acceptable salt thereof, and the APRIL binding antibody or antigen-binding fragment thereof may be co-administered with one or more alpha glucosidase inhibitors, such as miglitol (such as GLYSET™) or acarbose (such as PRECOSE™).

In some embodiments, an endothelin receptor antagonist, or a pharmaceutically acceptable salt thereof, and the APRIL binding antibody or antigen-binding fragment thereof may be co-administered with one or more biguanides, such as roseiglitazone (such as AVANDAMET™) or metformin (such as GLUCOPHAGE™ or GLUCOPHAGE XR™).

In some embodiments, an endothelin receptor antagonist, or a pharmaceutically acceptable salt thereof, and the APRIL binding antibody or antigen-binding fragment thereof may be co-administered with one or more insulins, such as HUMALOG™. HUMALOG 50/50™, HUMALOG 75/25™, HUMULIN 50/50™, HUMALIN 75/25™, HUMALIN L™, HUMALIN N™, HUMALIN R™, HUMALIN R U-500™, HUMALIN U™, ILETIN II LENTE™, ILETIN II NPH™, ILETIN II REGULAR™, LANTUS™, NOVOLIN 70/30™, NOVILIN N™, NOVILIN R™, NOVOLOG™, or VELOSULIN BR™, and EXUBERA™.

In some embodiments, an endothelin receptor antagonist, or a pharmaceutically acceptable salt thereof, and the APRIL binding antibody or antigen-binding fragment thereof may be co-administered with one or more meglitnides, such as repaglinide (such as PRANDIN™) or nateglinide (such as STARLIX™).

In some embodiments, an endothelin receptor antagonist, or a pharmaceutically acceptable salt thereof, and the APRIL binding antibody or antigen-binding fragment thereof may be co-administered with one or more sulfonylureas, such as glimepiride (such as AMARYL™), glyburide (such as DIABETA™, GLYNASE PRESTAB™ or MICRONASE™), or glipizide (such as GLUCOTROL™, or GLUCOTROL XL™).

In some embodiments, an endothelin receptor antagonist, or a pharmaceutically acceptable salt thereof, and the APRIL binding antibody or antigen-binding fragment thereof may be co-administered with one or more thiazolidinediones, such as pioglitazone (such as ACTOS™) or rosiglitazone (such as AVANDIA™).

In some embodiments, an endothelin receptor antagonist, or a pharmaceutically acceptable salt thereof, and the APRIL binding antibody or antigen-binding fragment thereof may be co-administered with niacin or one or more nicotinic acid derivatives, such as NIACOR™, NIASPAN™, NICOLAR™, or SLO-NIACIN™.

In some embodiments, an endothelin receptor antagonist, or a pharmaceutically acceptable salt thereof, and the APRIL binding antibody or antigen-binding fragment thereof may be co-administered with one or more fabric acid derivatives, such as clofibrate (such as ATROMID-S™), gemfibrozil (such as LOPID™), or fenofibrate (such as TRICOR™).

In some embodiments, an endothelin receptor antagonist, or a pharmaceutically acceptable salt thereof, and the APRIL binding antibody or antigen-binding fragment thereof may be co-administered with one or more bile acid sequestrants, such as colestipol (such as COLESTID™), cholestyramine (such as LOCHOLEST™, PREVALITE™, QUESTRAN™, or QUESTRAN LIGHT™), or colesevelam (such as WELCHOL™).

In some embodiments, an endothelin receptor antagonist, or a pharmaceutically acceptable salt thereof, and the APRIL binding antibody or antigen-binding fragment thereof may be co-administered with one or more cholesterol absorption inhibitors, such as ezetimibe (such as ZETIA™).

In some embodiments, an endothelin receptor antagonist, or a pharmaceutically acceptable salt thereof, and the APRIL binding antibody or antigen-binding fragment thereof may be co-administered with one or more 3-hydroxy-3-methylglutaryl coenzyme A (HMG-CoA) reductase inhibitors (statins) such as fluvastatin (such as LESCOL™), atorvastatin (such as LIPITOR™), lovastatin (such as ALTOCOR™ or MEVACOR™), pravastatin (such as PRAVACHOL™), rosuvastatin (such as CRESTOR™) simvastatin (such as ZOCOR™), or pitavastatin.

In some embodiments, an endothelin receptor antagonist, or a pharmaceutically acceptable salt thereof, and the APRIL binding antibody or antigen-binding fragment thereof may be co-administered with one or more additional agents. In some embodiments, the one or more additional agents is an immunosuppressant. In some embodiments, the one or more additional agents are selected from the group consisting of aminopterin, azathioprine, cyclosporin A, D-penicillamine, gold salts, hvdroxvchloroquine, leflunomide, methotrexate, minocycline, rapamycin, sulfasalazine, tacrolimus (FK506), and pharmaceutically acceptable salts thereof. As a non-limiting example, the one or more additional agents can be hydroxychloroquine.

In some embodiments, an endothelin receptor antagonist, or a pharmaceutically acceptable salt thereof, and the APRIL binding antibody or antigen-binding fragment thereof may be co-administered with one or more additional therapeutic agents selected group the group consisting of SGLT-2 inhibitor (such as canagliflozin), GR-immunosuppressant (such as budesonide), MASP-2 antibodies (such as OMS721), dual ETIA/ARB inhibitors (such as sparsentan). B cell modulators (e.g., APRIL modulators such as atacicept, APL-2, and VTS649), SYK inhibitor (such as fosamatinib), complement factor 3 convertase inhibitor (such as LNP023), NRF2 activator (such as Bardoxolone), and RNAi therapeutic targeting the C5 component of the complement pathway (e.g., cemdisiram).

In some embodiments, the one or more additional agents are SGLT-2 inhibitors. In some embodiments, the one or more additional agents is a SGLT-2 inhibitor selected from the group consisting of dapagliflozin, canagliflozin, ipragliflozin, empaglifozin, bexagliflozin, licogliflozin, janagliflozin (XZP-5695), tofogliflozin, ertugliflozin, henagliflozin (SHR-3824), enavogliflozin (DWP-16001), TA-1887 (3-(4-cyclopropylbenzyl)-4-fluoro-1-(Q-D-glucopyranosyl)-1H-indole), indole-N-glycoside 18 (3-(4-ethylbenzyl)-1-(D-D-glucopyranosyl)-1H-indole), sotagliflozin, luseogliflozin, sergliflozin etabonate, remogliflozin, remogliflozin etabonate, and T-1095 (((2R,3S,4S,5R,6S)-6-(2-(3-(benzofuran-5-yl)propanoyl)-3-hydroxy-5-methylphenoxy)-3,4,5-trihydroxytetrahydro-2H-pyran-2-yl) etabonate). In some embodiments, the one or more additional agents is a SGLT-2 inhibitor selected from the group consisting of bexagliflozin, canagliflozin, HM41322, dapagliflozin, empagliflozin, ertugliflozin, ipragliflozin, luseogliflozin, remogliflozin, serfliflozin, licofliglozin, sotagliflozin, and tofogliflozin. In some embodiments, the one or more additional agents is bexagliflozin. In some embodiments, the one or more additional agents is canagliflozin. In some embodiments, the one or more additional agents is dapagliflozin. In some embodiments, the one or more additional agents is empagliflozin. In some embodiments, the one or more additional agents is ertugliflozin. In some embodiments, the one or more additional agents is ipragliflozin. In some embodiments, the one or more additional agents is luseogliflozin. In some embodiments, the one or more additional agents is remogliflozin. In some embodiments, the one or more additional agents is serfliflozin. In some embodiments, the one or more additional agents is licofliglozin. In some embodiments, the one or more additional agents is sotagliflozin. In some embodiments, the one or more additional agents is tofogliflozin. In some embodiments, the SGLT-2 inhibitor is dapagliflozin propylene glycol hydrate. In some embodiments, the SGLT-2 inhibitor is canagliflozin hemihydrate.

In some embodiments, the amount of the SGLT-2 inhibitor is from about 1 mg to about 350 mg. For example, about 1 mg to about 175 mg, about 175 mg to about 350 mg, or about 90 mg to about 260 mg. In some embodiments, the amount of the SGLT-2 inhibitor is from about 85 mg to about 325 mg. In some embodiments, the amount of the SGLT-2 inhibitor is from about 1 mg to about 50 mg, about 20 mg to about 70 mg, about 50 mg to about 100 mg, about 70 mg to about 120 mg, about 90 mg to about 140 mg, about 110 mg to about 160 mg, about 130 mg to about 180 mg, about 150 mg to about 200 mg, about 170 mg to about 220 mg, about 190 mg to about 240 mg, about 210 mg to about 260 mg, about 230 mg to about 280 mg, about 250 mg to about 300 mg, about 270 mg to about 320 mg, or about 290 mg to about 350 mg. For example, about 100 mg or about 300 mg. In some embodiments, the amount of the SGLT-2 inhibitor is from about 1 to about 15 mg. For example, about 1 to about 10 mg or about 5 to about 15 mg. In some embodiments, the amount of the SGLT-2 inhibitor is from 1 mg to about 3 mg, about 2 mg to about 4 mg, about 3 mg to about 5 mg, about 4 mg to about 6 mg, about 5 mg to about 7 mg, about 6 mg to about 8 mg, about 7 mg to about 9 mg, about 8 mg to about 10 mg, about 9 mg to about 11 mg, about 10 mg to about 12 mg, about 11 mg to about 13 mg, about 12 mg to about 14 mg, or about 13 mg to about 15 mg.

In one embodiment of the methods or uses or product for uses provided herein, a SGLT-2 inhibitor, or a pharmaceutically acceptable salt or solvate thereof, is administered to the subject daily.

In some embodiments, the SGLT-2 inhibitor is canagliflozin. In some embodiments. 100 mg or 300 mg of canagliflozin is administered. In some embodiments, 100 mg or 300 mg of canagliflozin hemihydrate is administered. In some embodiments, the SGLT-2 inhibitor is dapagliflozin. In some embodiments, the SGLT-2 inhibitor is dapagliflozin propylene glycol hydrate. In some embodiments, 5 mg or 10 mg of dapagliflozin is administered. In some embodiments, 5 mg or 10 mg of dapagliflozin propylene glycol hydrate is administered. In some embodiments, the SGLT-2 inhibitor is empagliflozin. In some embodiments, 10 mg or 25 mg of empagliflozin is administered. In some embodiments, the SGLT-2 inhibitor is ertugliflozin. In some embodiments, 5 mg or 15 mg of ertugliflozin is administered. In some embodiments, the SGLT-2 inhibitor is ipragliflozin. In some embodiments, 25 mg or 50 mg of ipragliflozin is administered. In some embodiments, the SGLT-2 inhibitor is bexagliflozin. In some embodiments, 20 mg of bexagliflozin is administered. In some embodiments, the SGLT-2 inhibitor is sotagliflozin. In some embodiments, 200 mg or 400 mg of sotagliflozin is administered. In some embodiments, the SGLT-2 inhibitor is licogliflozin. In some embodiments, 15 mg, 50 mg, 75 mg or 150 mg of licogliflozin is administered.

In any of the embodiments described herein, various combinations of an endothelin receptor antagonist, or a pharmaceutically acceptable salt thereof, and the APRIL binding antibody or antigen-binding fragment thereof, and a SGLT-2 inhibitor, producing an effect, are contemplated. In some embodiments, the effect, for example, any of the beneficial or desired results as described herein, is greater than the sum of the effect observed when the same amount of an endothelin receptor antagonist, or a pharmaceutically acceptable salt thereof, and the APRIL binding antibody or antigen-binding fragment thereof, when co-administered, and the same amount of the SGLT-2 inhibitor when co-administered, are administered as a monotherapy. In some embodiments, the co-administration of an endothelin receptor antagonist, or a pharmaceutically acceptable salt thereof, and the APRIL binding antibody or antigen-binding fragment thereof, and a SGLT-2 inhibitor, produce an effect, for example, a therapeutic effect using a smaller dose of either, or both, of the compounds as a monotherapy. For example, producing a therapeutic effect using a smaller dose of an endothelin receptor antagonist, or a pharmaceutically acceptable salt thereof, and the APRIL binding antibody or antigen-binding fragment thereof, and/or the SGLT-2 inhibitor compared to the amount used in monotherapy. For example, in some embodiments, the dose of an endothelin receptor antagonist, or a pharmaceutically acceptable salt thereof, and the APRIL binding antibody or antigen-binding fragment thereof, administered in combination with a SGLT-2 inhibitor may be about 50% to about 90% of the dose of an endothelin receptor antagonist, or a pharmaceutically acceptable salt thereof, and the APRIL binding antibody or antigen-binding fragment thereof, administered as a monotherapy to produce the same therapeutic effect, e.g., any of the beneficial or desired results including described herein. In some embodiments, the dose of the SGLT-2 inhibitor, administered in combination with an endothelin receptor antagonist, or a pharmaceutically acceptable salt thereof, and the APRIL binding antibody or antigen-binding fragment thereof, may be about 50% to about 90% of the dose of the SGLT-2 inhibitor, administered as a monotherapy to produce the same therapeutic effect, e.g., any of the beneficial or desired results including described herein. For example, treating IgA nephropathy, decreasing renal inflammation and/or fibrosis, decreasing hematuria, decreasing proteinuria, stabilizing eGFR, decreasing the number of IgA-nephropathy associated disease flares, delaying the onset of ESRD, decreasing fatigue, and reducing activation of a mesangial cell.

In some embodiments, the present disclosure relates to the use of an endothelin receptor antagonist, or a pharmaceutically acceptable salt thereof, and the APRIL binding antibody or antigen-binding fragment thereof in combination with a second therapeutic for treating a condition as described in the various embodiments of the disclosure.

In some embodiments, the present disclosure relates to the use of an endothelin receptor antagonist, or a pharmaceutically acceptable salt thereof, and the APRIL binding antibody or antigen-binding fragment thereof, for treating a condition as described in the various embodiments of the disclosure, wherein the use comprises one or more additional therapeutic agent.

In some embodiments, the present disclosure relates to a pharmaceutical composition comprising an endothelin receptor antagonist, or a pharmaceutically acceptable salt thereof, and the APRIL binding antibody or antigen-binding fragment thereof, and further comprising one or more additional therapeutic agent.

In some embodiments, the one or more additional therapeutic agent inhibits one or more elements of the renin-angiotensin-aldosterone system. In some embodiments, the one or more additional therapeutic agent is selected from the group consisting of diuretics, angiotensin converting enzyme (ACE) inhibitors, angiotensin II receptor (ARB) blockers, calcium channel blockers, renin inhibitors, and aldosterone antagonists. In certain particular embodiments, the one or more additional therapeutic agent is selected from the group consisting of angiotensin converting enzyme (ACE) inhibitors and angiotensin II receptor blockers (ARBs). In certain embodiments, the one or more additional therapeutic agent is selected from one or more angiotensin converting enzyme inhibitors. In certain embodiments, the one or more additional therapeutic agent is selected from one or more angiotensin II receptor blockers. In certain embodiments, the one or more additional therapeutic agent comprises one or more ACE inhibitors and one or more ARBs. For example, the one or more inhibitors of the renin-angiotensin system can be ACE inhibitor, ARB, or a combination thereof. For example, the ACE inhibitor can be selected from the group consisting of quinapril, fosinopril perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril. For example, the ARB can be selected from the group consisting of candesartan, candesartan cilexetil, eprosartan, irbesartan, losartan, olmesartan, olmesartan medoxomil, telmisartan, valsartan, azilsartan medoxomil, and BRA-657.

In some embodiments, an endothelin receptor antagonist, or a pharmaceutically acceptable salt thereof, and the APRIL binding antibody or antigen-binding fragment thereof, may be co-administered with a SGLT-2 inhibitor and one or more ACE inhibitors and/or one or more ARBs. In some embodiments, an endothelin receptor antagonist, or a pharmaceutically acceptable salt thereof, and the APRIL binding antibody or antigen-binding fragment thereof, may be co-administered with a SGLT-2 inhibitor and one or more ACE inhibitors. In some embodiments, an endothelin receptor antagonist, or a pharmaceutically acceptable salt thereof, and the APRIL binding antibody or antigen-binding fragment thereof, may be co-administered with a SGLT-2 inhibitor and one or more ARBs. In some embodiments, an endothelin receptor antagonist, or a pharmaceutically acceptable salt thereof, and the APRIL binding antibody or antigen-binding fragment thereof, may be co-administered with a SGLT-2 inhibitor, an ACE inhibitor, and an ARB.

It is understood that the examples and embodiments described herein are for illustrative purposes only and that various modifications or changes in light thereof will be suggested to persons skilled in the art and are to be included within the spirit and purview of this application and scope of the appended claims.

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES AND PROPHETIC EXAMPLES

### Prophetic Example 1. In Vitro Study Using a Cellular Model of Human IgA Nephropathy

Primary human mesangial cells in a culture are stimulated with pathogenic dgIgA immune complexes isolated from human IgA nephropathy patients or generated in vitro. Proliferation as well as pro-inflammatory and pro-fibrotic responses to these disease causing immune complexes are observed in mesangial cells within 48 hours. The cells are treated with an endothelin receptor antagonist, or a pharmaceutically acceptable salt thereof, and the APRIL binding antibody or antigen-binding fragment thereof (e.g., atrasentan hydrochloride) in an appropriate medium. Changes in proliferation, pro-inflammatory responses, and/or pro-fibrotic responses are measured. The result of this study will indicate the extent to which atrasentan attenuates underlying disease processes in IgA nephropathy in an in vitro model.

### Example 2. A Phase 1/2, Randomized, Double-blind, Placebo-controlled Study of BION-1301 in Adults with IgA Nephropathy

IgA nephropathy is a chronic, autoimmune, inflammatory glomerulopathy characterized by a multi-hit disease pathogenesis (Lai 2016; Suzuki 2021). The excess production of galactose-deficient IgA1 (Gd-IgAQ1) by IgA-secreting plasma cells is considered the initiating pathogenic event (Hit 1) in IgA nephropathy. The glomerular immune-deposits in that are diagnostic for IgAN on biopsy by immunofluorescence microscopy are heavily enriched for aberrantly glycosylated IgAQ1 glycoforms (Gd-IgAQ1), deficient in galactose residues in the hinge region. This deficiency of glycosylation of the IgAQ1 hinge can be genetically determined or induced as part of the dysregulated mucosal immune response thought to trigger IgAN commonly following a mucosal infection. Gd-IgAQ1 itself does not appear to be pathogenic, but in susceptible individuals, immune recognition of GdIgA1 by circulating anti-glycan, typically IgG auto-antibodies (Hit 2) leads to the formation of Gd-IgA1 immune complexes (Hit 3). These immune complexes drive renal injury following glomerular deposition. This stimulates mesangial cell activation, and secretion of cytokines, chemokines, and extracellular matrix proteins. This ultimately results in inflammation and fibrosis (Hit 4). Therefore, agents that act on the initiating molecular events that drive excess Gd-IgA1 production represent targeted and potentially disease modifying approaches in the treatment of IgAN.

BION-1301 is a novel humanized IgG4 monoclonal antibody that binds the soluble ligand a proliferation-inducing ligand (APRIL) and effectively blocks the interaction of APRIL with its receptors, B-cell maturation antigen (BCMA), and transmembrane activator calcium modulator and cyclophilin ligand interactor (TACI) on lymphocytes (Guadagnoli 2011). APRIL is a TNF-super-family cytokine member that has been reported to promote excess secretion of Gd-IgAQ1 in IgAN (Zhai 2016). Therefore, BION-1301 provides a potentially disease-modifying approach to the treatment of IgAN by directly targeting the disease pathogenesis through its effect to deplete Gd-IgAQ1 (Hit 1) and prevent the pathogenic immune complex formation (Hit 3) that drives kidney injury following glomerular deposition (Hit 4).

The plasma cell, considered the pathogenic target cell in IgAN, is responsible for the secretion of both Gd-IgAQ1 and the IgG auto-antibody. APRIL receptors BCMA and TACI are highly expressed on plasma cells, suggesting the potential for APRIL modulation to impact the key target cell in IgAN. In addition, TACI and BCMA expression are restricted to the plasma cell and plasmablasts, with lower levels on memory B cells and no expression on earlier stage lymphocytes. For these reasons, APRIL inhibition is anticipated to be immunomodulatory, but not to be broadly immunosuppressive.

BION-1301 has been studied in adults with relapsed or refractory multiple myeloma as well as healthy volunteers (HVs) and adults with IgAN. Since the initiation of the BION-1301 clinical program through 26 Jul. 2022, a total of 153 adults have been enrolled in 5 clinical studies. Interim results from an ongoing Phase 1/2 clinical study evaluating IV administration of BION-1301 450 mg Q2W in patients with IgAN demonstrated that BION-1301 treatment was well-tolerated and resulted in sustained reductions in free (unbound) APRIL, Gd-IgA1, and proteinuria (as measured by areduction in UPCR). Following IV infusion, serum BION-1301 concentrations appeared comparable to those observed in healthy volunteers at the same dose and schedule. Following at least 24-weeks of IV administration, all subjects who transitioned to SC dosing at 600 mg Q2W maintained similar BION-1301 exposures, biomarker responses and proteinuria reductions. On average, patients treated with BION-1301 demonstrated a reduction in 24-hour UPCR which was evident by 3 months and continue to decline beyond 1 year, thereby providing preliminary clinical evidence of efficacy. The magnitude of the proteinuria reductions observed are predicted to translate into clinically meaningful preservation of eGFR and significantly improved long-term kidney outcomes (Inker 2021: Thompson 2019).

### Prophetic Example 3. Continuation of Phase 3, Randomized, Double-blind, Placebo-controlled Study of BION-1301 in Adults with IgA Nephropathy

The current planned study will further evaluate the efficacy and safety of BION-1301 in adults with IgAN who remain at risk of progression despite optimal treatment with angiotensin-converting enzyme inhibitor (ACEi)/angiotensin II receptor blocker (ARB), additionally, as newer therapies gain approval and are used more frequently in this population, the Phase 3 study will not exclude patients who are treated with sodium-glucose cotransporter-2 (SGLT-2) inhibitors, mineralocorticoid antagonist (MRAs), and endothelin receptor antagonist, or a pharmaceutically acceptable salt thereofs (ERAs) as background therapy.

Approximately 136 subjects with IgAN per arm are planned. An additional exploratory cohort, which will not be included in the primary analysis population, will be comprised of 20 subjects (10 subjects per arm) with biopsy-confirmed IgAN and eGFR of 20 to <30 mL/min/1.73 m² to evaluate the effect of BION-1301 versus placebo on proteinuria.

### Study Design

CHK02-02 is a Phase 3, randomized, double-blind, placebo-controlled study in adults with primary immunoglobulin A nephropathy (IgAN) at risk of progressive kidney function loss. Randomization is stratified by region (Asia vs. Rest of World), baseline proteinuria (≥2 g/day vs. <2 g/day), and eGFR (<45 ml/min/1.73^{m} vs. >45 ml/min/1.73 m²).

Subjects are eligible to participate if they are >18 years of age and have biopsy-proven IgAN and meet all other eligibility criteria. For all subjects, the study is comprised of a Screening Period and a double-blinded Treatment Period. Following the Screening Period (Days−42 to−1), subjects will be randomized 1:1 on Day 1 (baseline) to receive subcutaneous (SC) doses of either BION-1301 or matching placebo during a 104-week Treatment Period. Study drug will be administered every 2 weeks (Q2W) during the Treatment Period. Subjects will then enter a 24-week safety Follow-up period.

Subjects who prematurely discontinue study drug before Week 104 will be asked to remain in the study. These subjects who prematurely discontinue will have an End of Treatment (EoT) visit at the time of study drug discontinuation, followed by a visit in 4 weeks and then quarterly assessments for efficacy and safety through Week 104.

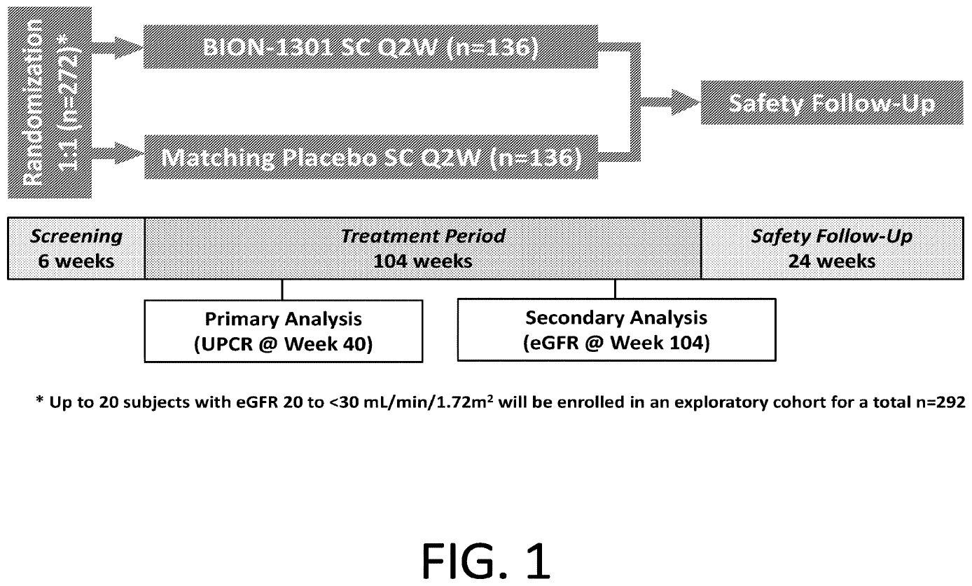

An independent data monitoring committee (IDMC) will be appointed to monitor the study to ensure the best interests and safety of the study subjects as well as evaluate the continuing validity and scientific merit of the study. The study schema is presented in FIG. 1, and objectives and endpoints of this study are provided in Table 2.

TABLE 2
Objectives and Endpoints.

| Objectives | Endpoints |
|---|---|
| Primary | |
| Evaluate the effect of BION-1301 | Change in proteinuria (urine protein:creatinine |
| versus placebo on proteinuria | ratio [UPCR]) based on 24-hour urine collection |
| | from baseline to Week 40 |
| Key Secondary | |
| Evaluate the effect of BION-1301 | Change from baseline to the final study visit |
| versus placebo on estimated | (Week 104) in eGFR using the chronic kidney |
| glomerular filtration rate (eGFR) | disease-epidemiology collaboration (CKD-EPI |
| | 2021) creatinine equation. |
| Secondary | |
| Additional efficacy outcomes | Percent of subjects meeting the composite |
| | endpoint of experiencing at least one of the |
| | following during the study: |
| | At least 30% reduction in eGFR |
| | sustained for at least 30 days |
| | eGFR <15 mL/min/1.73 m², sustained |
| | for at least 30 days |
| | Chronic dialysis, ≥30 days |
| | Kidney transplantation |
| | All-cause mortality |
| | Percent of subjects meeting the composite |
| | endpoint of experiencing at least one of the |
| | following during the study: |
| | At least 40% reduction in eGFR |
| | sustained for at least 30 days |
| | eGFR <15 mL/min/1.73 m², sustained |
| | for at least 30 days |
| | Chronic dialysis, ≥30 days |
| | Kidney transplantation |
| | All-cause mortality |
| | Percent of subjects achieving reduction of |
| | proteinuria to <1.0 g/day at Week 40 and |
| | a ≥25% decrease in total urine protein from |
| | Baseline |
| Exploratory/Tertiary | |
| Further characterize the safety and | Type, incidence, severity, and relatedness of |
| tolerability of BION-1301compared to | adverse events (AEs) and serious adverse events |
| placebo | (SAEs) |
| | Changes in other safety parameters |
| Characterize the pharmacokinetics of | Serum concentrations of BION-1301 |
| BION-1301 and to support | |
| exploratory exposure-response | |
| analyses | |
| Characterize the immunogenicity of | Presence of circulating binding and neutralizing |
| BION-1301 | anti-drug antibodies (ADA/NAb) |
| Evaluate the impact of BION-1301 on | Change from baseline over time in European |
| health-related quality of life (HRQOL) | Quality of Life 5 dimensions 5 levels (EQ-5D- |
| | 5L) and in Kidney Disease Quality of Life-36 |
| | items (KDQOL-36 ™) |
| Evaluate the mechanism of action of | Change from baseline in blood and urine |
| BION-1301 and IgAN disease | biomarkers |
| processes | Research assessment of naturally occurring |
| | genetic variation (pharmacogenetics) |
| Evaluate long-term impact of BION- | Change from baseline in vaccine titres and |
| 1301 on vaccine titres, responses and | immune cell phenotypes |
| immune cell phenotypes | |
| Evaluate the effect of BION-1301 | Change in proteinuria (urine protein:creatinine |
| versus placebo on proteinuria in | ratio [UPCR]) based on 24-hour urine collection |
| patients with eGFR of 20 to <30 | from baseline to Week 40 |
| mL/min/1.73 m² | |

### Inclusion Criteria

Subjects must meet all of the following criteria to be included in the study.

### Age and Sex:

1. Male and female subjects aged >18 years at the time of signing the informed consent form (ICF) prior to initiation of any study specific activities/procedures.

### Type of Subjects and Disease Characteristics:

2. Biopsy-confirmed IgAN. The biopsy could have occurred at any point in time prior to the study and the diagnostic report must be available for review by the Sponsor or designee.
3. eGFR >30 ml/min/1.73 m² at Screening based on the CKD-EPI equation (for the exploratory cohort only: eGFR 20 to <30 mL/min/1.73 m²)
4. Total urine protein ≥1.0 g/day as measured by a central laboratory via a 24-hour urine collection collected at Screening
5. Stable on a maximally tolerated dose of ACEi/ARB for at least 12 weeks prior to Screening or intolerant to ACEi/ARB; may also be on a stable and maximally tolerated dose of SGLT-2i and/or ERAs/MRAs for at least 12 weeks prior to Screening
6. Body mass index (BMI) between 18 and 40 kg/m², inclusive, at Screening with a weight of at least 50 kg
7. Average blood pressure (BP)<140/90 mmHg (systolic/diastolic) from 2 readings obtained after the subjects has rested in a seated position with back supported for 5 minutes at the initial Screening visit.

### Exclusion Criteria

Subjects will be excluded if they meet any of the following criteria.
1. Secondary forms of IgAN as determined by the Investigator, in the setting of systemic disorders, infections, autoimmune disorders or neoplasias.
2. Diagnosis of IgAV-associated nephritis.
3. Clinical diagnosis of Nephrotic Syndrome.
4. Clinical suspicion of IgAN with rapidly progressive glomerulonephritis (RPGN) based on KDIGO guidelines (KDIGO 2021).
5. Chronic kidney disease resulting from any condition other than IgAN.
6. Diagnosis of Type 1 or Type 2 diabetes.
7. Prior exposure to any antibody directed against APRIL.
8. Known or suspected allergy or hypersensitivity to any component of BION-1301, or history of severe hypersensitivity reaction to any monoclonal antibody.
9. Received an investigational new drug, or investigational device within 28 days (or 5 half-lives, whichever is longer) prior to Screening.
10. Received systemic corticosteroid therapy (average >20 mg/day of prednisone or equivalent for at least 14 days) within 12 weeks prior to Screening.
11. Use of systemic immunosuppressant medications including systemic corticosteroids (e.g., prednisone, prednisolone, Tarpeyo, etc.), mycophenolate, azathioprine, cyclosporine, tacrolimus, cyclophosphamide etc.; use of herbs such as Tripterygium Wilfordii Hook F, Caulis sinomenii and Sinomenium acutum; for >2 weeks in the past 12 weeks prior to Screening. Use of rituximab within the past 6 months prior to Screening.
12. Any confirmed or suspected immunosuppressive or immune-deficient state, including but not limited to HIV infection or asplenia
13. Current infection or history of recurrent, severe, infections.
14. Positive serology test for hepatitis A virus IgM antibodies (anti-HAV IgM), hepatitis B surface antigen (HBsAg), hepatitis C virus (HCV) antibodies, or antibodies to HIV-1 and/or HIV-2 at Screening.
15. Positive QuantiFERON-TB Gold Plus test.
16. Received a live vaccination within 12 weeks prior to Screening or plan to have a live vaccination within 6 months after the last dose of study drug.
17. History of malignancy unless cancer free for at least 5 years or non-melanoma skin cancer that was completely resected. A subject with curatively treated cervical carcinoma in situ is eligible for the study. Patients with low-risk prostate cancer (ie, Gleason score <7 and prostate specific antigen <10 ng/mL) are allowed.
18. Pregnancy or breastfeeding or intent to become pregnant during the study period.
19. History or evidence of any other clinically significant disorder, condition, disease, or laboratory finding that would place the subject at unacceptable risk, limit compliance with study requirements, or confound interpretation of study results.
20. IgG levels <6 g/L at Screening per central laboratory reference ranges.
21. Have received any investigational agent (including monoclonal antibodies) within 4 weeks (or 5 half-lives of the agent, whichever is longer) prior to Screening.
22. Participation in another interventional trial with an investigational agent is prohibited during the course of this study.

Duration of Participation: Total duration for subjects who complete the study is expected to be approximately 134 weeks (screening: up to 6 weeks; treatment (blinded): 104 weeks; follow-up period: 24 weeks).

Study Drug: Study drug includes both BION-1301 and matching placebo as shown in Table 3.

TABLE 3
Study Drug

| ARM Name | Active | Control |
|---|---|---|
| Drug Name | BION-1301 | Placebo |
| Type | Drug (monoclonal antibody) | NA |
| Dose Formulation | Solution | Solution |
| Unit Dose Strength(s) | 150 mg/mL | NA |
| Route of | Subcutaneous Injection via pre- | Subcutaneous |
| Administration | filled syringe (PFS) with | Injection via |
| | needle safety device (NSD) | PFS with NSD |

### Statistical Methodology

Sample Size: A target of approximately 272 subjects will be randomized 1:1 to receive BION-1301 600 mg Q2W or a matched placebo for 104 weeks. An additional 20 subjects with eGFR 20 to <30 mL/min/1.73 m² will be enrolled into an exploratory cohort and not included in the primary or secondary analyses, yielding a total randomized sample size of 292.

For the primary endpoint of UPCR change from baseline, approximately 204 patients will provide 90% power to detect at least a 35% placebo-adjusted treatment effect (log-transformed value of 0.43) in the BION-1301 group compared to placebo with a two-sided alpha of 0.01 assuming a standard deviation of the log-transformed value of 0.75 and a 10% rate of early study discontinuation prior to Week 40. A two-sample t-test was used to calculate sample size however the primary efficacy analysis will be conducted using a mixed-model repeated measures (MMRM) approach. In general, the MMRM should result in an increase of power over the t-test in the presence of missing data.

For the key secondary endpoint of eGFR change from baseline, approximately 272 patients will provide 90% power to detect least a 3.0 mL/min/1.73 m² difference (ie, 1.5 mL/min/1.73 m²/year) in mean eGFR change from baseline at Week 104 between BION-1301 and placebo with a two-sided alpha of 0.05 assuming a standard deviation of 7.0 and a 15% rate of early study discontinuation.

### Analysis Populations

Four analysis populations are planned for this study, as shown in Table 4.

TABLE 4
Analysis Populations

| Analysis Population | Definition |
|---|---|
| Intent-to-treat (ITT) | All subjects randomly assigned to study drug. Subjects will be |
| | analyzed according to the intervention to which they have been |
| | randomly assigned. |
| Per Protocol | Subjects in the ITT population who completed all study visits |
| | and had no major protocol deviations |
| Safety Population | All enrolled participants who receive any amount of BION- |
| | 1301 or placebo comprise the Safety Population for safety and |
| | exposure analyses. |
| Pharmacokinetic (PK) | All enrolled participants who receive any amount of BION- |
| Population | 1301 and who have at least one post-baseline PK sample will |
| | comprise the PK Population. |

### Statistical Methodology

Age and Sex Efficacy Analyses: The primary efficacy endpoint is the change from baseline at Week 40 in urinary protein to creatinine ratio (UPCR) as determined from 24-hour urine collection samples. The primary analysis will be conducted after approximately 204 subjects in the ITT analysis set reach Week 40 or discontinue the study and will be tested based on a 2-sided significance level of 0.01. The primary endpoint will be analyzed using a mixed-effects model repeated-measures (MMRM) model. The MMRM model will include change from Baseline of natural log UPCR at each post-Baseline measurement as outcomes. The model will also include the fixed effects of treatment, visit, and treatment-by-visit interaction, with covariates of baseline natural log UPCR and baseline eGFR as continuous variables and region randomization stratification factors (region: Asia vs all other regions).

Sensitivity analyses of the primary endpoint will be conducted to assess the robustness of the results of primary analysis and to evaluate the impact of missing data and the missing at random assumption of the MMRM model. These sensitivity analyses include the following:
Primary endpoint analysis based on MMRM without censoring (includes all on- and off-treatment UPCR values through Week 40)
UPCR change from baseline at Week 40 based on ANCOVA methodology
Primary Endpoint Tipping Point analysis
Primary Endpoint Copy Reference Multiple Imputation Method assuming the missing data in the treatment group will have a profile that equals the profile of the control group for all time points

One skilled in the art readily appreciates that the present disclosure is well adapted to carry out the objects and obtain the ends and advantages mentioned, as well as those inherent therein. The examples provided herein are representative of preferred embodiments, are exemplary, and are not intended as limitations on the scope of the disclosure.

It is to be understood that the disclosure is not limited in its application to the details of construction and to the arrangements of the components set forth in the following description or illustrated in the drawings. The disclosure is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present disclosure. It is important, therefore, that the claims be regarded as including such equivalent constructions insofar as they do not depart from the spirit and scope of the present disclosure.

While the disclosure has been described and exemplified in sufficient detail for those skilled in this art to make and use it, various alternatives, modifications, and improvements should be apparent without departing from the spirit and scope of the disclosure. The examples provided herein are representative of preferred embodiments, are exemplary, and are not intended as limitations on the scope of the disclosure. Modifications therein and other uses will occur to those skilled in the art. These modifications are encompassed within the spirit of the disclosure and are defined by the scope of the claims.

It will be readily apparent to a person skilled in the art that varying substitutions and modifications may be made to the disclosure described herein without departing from the scope and spirit of the disclosure.

Unless otherwise defined, all technical and scientific terms used herein have the same meaning as commonly understood by one of ordinary skill in the art to which this disclosure belongs. In the case of conflict, the present specification, including definitions, will control.

The term “about”, “approximately”, or “approximate”, when used in connection with a numerical value, means that a collection or range of values is included. For example, “about X” includes a range of values that are ±20%, ±10%, ±5%, ±2%, ±1%, ±0.5%, +0.2%, or ±0.10% of X, where X is a numerical value. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the terms “between X and Y” and “range from X to Y, are inclusive of X and Y and the integers there between. On the other hand, when a series of individual values are referred to in the disclosure, any range including any of the two individual values as the two end points is also conceived in this disclosure. For example, the expression “a dose of about 100 mg, 200 mg, or 400 mg” can also mean “a dose ranging from 100 to 200 mg”, “a dose ranging from 200 to 400 mg”, or “a dose ranging from 100 to 400 mg”.

The disclosure described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

Other embodiments are set forth within the following claims.

1. A method of treating IgA nephropathy in a subject, comprising administering to the subject an endothelin receptor antagonist, or a pharmaceutically acceptable salt thereof, and an APRIL binding antibody or an antigen-binding fragment thereof.

2. A method of decreasing renal inflammation and/or fibrosis in a subject having IgA nephropathy, comprising administering to the subject an endothelin receptor antagonist, or a pharmaceutically acceptable salt thereof, and an APRIL binding antibody or an antigen-binding fragment thereof.

3. A method of decreasing the occurrence of hematuria in a subject having IgA nephropathy, comprising administering to the subject an endothelin receptor antagonist, or a pharmaceutically acceptable salt thereof, and an APRIL binding antibody or an antigen-binding fragment thereof.

4. A method of stabilizing eGFR in a subject having IgA nephropathy, comprising administering to the subject an endothelin receptor antagonist, or a pharmaceutically acceptable salt thereof, and an APRIL binding antibody or an antigen-binding fragment thereof.

5. The method according to claim 1, wherein the endothelin receptor antagonist, or a pharmaceutically acceptable salt thereof, is selected from the group consisting of tezosentan, spysentan, bosentan, sparsentan, macitentan, ambrisentan, sitaxentan, atriopeptine, atrasentan, and pharmaceutically acceptable salts of any of the foregoing, and combinations thereof.

6. The method according to claim 1, wherein the endothelin receptor antagonist, or a pharmaceutically acceptable salt thereof, is sparsentan, or a pharmaceutically acceptable salt thereof.

7. The method according to claim 1, wherein the endothelin receptor antagonist, or a pharmaceutically acceptable salt thereof, is atrasentan, or a pharmaceutically acceptable salt thereof.

8. (canceled)

9. The method according to claim 7, wherein atrasentan, or a pharmaceutically acceptable salt thereof, is administered to the subject in an amount equivalent to about 0.20 mg to about 1.50 mg of atrasentan free base.

10. The method according to claim 9, wherein atrasentan, or a pharmaceutically acceptable salt thereof, is administered to the subject in an amount equivalent to about 0.75 mg of atrasentan free base.

11. The method according to claim 7, wherein the pharmaceutically acceptable salt of atrasentan is atrasentan hydrochloride or atrasentan mandelate.

12. (canceled)

13. (canceled)

14. (canceled)

15. The method according to claim 1, wherein the APRIL binding antibody or antigen-binding fragment thereof comprises:
(a) a heavy chain complementarity-determining region-1 (HC CDR1) comprising an amino acid sequence of SEQ ID NO:1;
(b) a heavy chain complementarity-determining region-2 (HC CDR2) comprising an amino acid sequence of SEQ ID NO:2;
(c) a heavy chain complementarity-determining region-3 (HC CDR3) comprising an amino acid sequence of SEQ ID NO:3;
(d) a light chain complementarity-determining region-1 (LC CDR1) comprising an amino acid sequence of SEQ ID NO:4;
(e) a light chain complementarity-determining region-2 (LC CDR2) comprising an amino acid sequence of SEQ ID NO:5; and
(f) a light chain complementarity-determining region-3 (LC CDR3) comprising an amino acid sequence of SEQ ID NO:6.

16. The method according to any one of claim 1, wherein the APRIL binding antibody or antigen-binding fragment thereof comprises a heavy chain variable region comprising an amino acid sequence having at least 90% sequence identity to SEQ ID NOs: 8, 10, 12, 14, 16, 18, 20, 22, or 24.

17. The method according to claim 1, wherein the APRIL binding antibody or antigen-binding fragment thereof comprises a light chain variable region comprising an amino acid sequence having at least 90% sequence identity to SEQ ID NO: 26.

18. (canceled)

19. The method according to claim 1, wherein the APRIL binding antibody or antigen-binding fragment thereof comprises a heavy chain comprising an amino acid sequence having at least 90% sequence identity to SEQ ID NO: 28.

20. The method according to claim 1, wherein the APRIL binding antibody or antigen-binding fragment thereof comprises a light chain comprising an amino acid sequence having at least 90% sequence identity to SEQ ID NO: 30.

21. (canceled)

22. The method according to claim 1, wherein the APRIL binding antibody or antigen-binding fragment thereof is generated from a nucleic acid comprising one of SEQ ID NOs: 7, 9, 11, 13, 15, 17, 19, 21, or 23.

23. The method according to claim 1, wherein the APRIL binding antibody or antigen-binding fragment thereof is generated from a nucleic acid comprising SEQ ID NO: 25, 27, 29, 31, or 33.

24. (canceled)

25. (canceled)

26. The method according to claim 1, wherein the method comprises repeating the administration of the APRIL binding antibody or antigen-binding fragment thereof on at least an every week (QW) schedule for at least 2 administration cycles; or
wherein the method comprises repeating the administration of the APRIL binding antibody or antigen-binding fragment thereof on at least an every two weeks (Q2W) schedule for at least 2 administration cycles; or
wherein the method comprises repeating the administration of the APRIL binding antibody or antigen-binding fragment thereof on at least an every 4 weeks (Q4W) or monthly (QMT) schedule for at least 2 administration cycles.

27. The method according to claim 26, comprising administering the APRIL binding antibody or antigen-binding fragment thereof by a loading/maintenance administration protocol.

28. The method according to claim 1, wherein the subject is concomitantly receiving an angiotensin converting enzyme (ACE) inhibitor, an angiotensin II receptor blocker (ARB), or a combination thereof.

29. The method according to claim 1, further comprising administering a therapeutically effective amount of a sodium-glucose co-transporter-2 (SGLT-2) inhibitor.

30. A kit comprising:
endothelin receptor antagonist, or pharmaceutical salt thereof, and an APRIL binding antibody or an antigen-binding fragment thereof, wherein the endothelin receptor antagonist, or pharmaceutical salt thereof, and the APRIL binding antibody or antigen-binding fragment thereof can be in the same and/or separate dosage form.
